SEQUENCE LISTING (149 sequences)

SEQ ID NO: 1 - DNA, 25 bp, Artificial Sequence
  Description of Artificial Sequence Synthetic primer
gtttcccagtaggtctcnnnnnnnn

SEQ ID NO: 2 - DNA, 21 bp, Artificial Sequence
  Description of Artificial Sequence Synthetic primer
cgccgtttcccagtaggtctc

SEQ ID NO: 3 - DNA, 27 bp, Artificial Sequence
  Description of Artificial Sequence Synthetic primer
aacaccgggtcttaattcttatatcaa

SEQ ID NO: 4 - DNA, 28 bp, Artificial Sequence
  Description of Artificial Sequence Synthetic primer
ggtggtaaaattcccatagtagttcttt

SEQ ID NO: 5 - DNA, 23 bp, Artificial Sequence
  Description of Artificial Sequence Synthetic primer
agaacacgtgccgcttacgccca

SEQ ID NO: 6 - DNA, 27 bp, Artificial Sequence
  Description of Artificial Sequence Synthetic primer
ccattcyttyttraactcytcaaacca

SEQ ID NO: 7 - DNA, 20 bp, Artificial Sequence
  Description of Artificial Sequence Synthetic primer
caggattgcagcagggaaga

SEQ ID NO: 8 - DNA, 21 bp, Artificial Sequence
  Description of Artificial Sequence Synthetic primer
atgatcaccaggytctacccc

SEQ ID NO: 9 - DNA, 20 bp, Artificial Sequence
  Description of Artificial Sequence Synthetic primer
tggaagcctggctgaaagag

SEQ ID NO: 10 - DNA, 20 bp, Artificial Sequence
  Description of Artificial Sequence Synthetic primer
tcatccccactgaccagcat

SEQ ID NO: 11 - DNA, 21 bp, Artificial Sequence
  Description of Artificial Sequence Synthetic primer
ggattgacctgtgcctgttgc

SEQ ID NO: 12 - DNA, 26 bp, Artificial Sequence
  Description of Artificial Sequence Synthetic primer
gcattagaaatgtcctcttttgctgc

SEQ ID NO: 13 - DNA, 25 bp, Artificial Sequence
  Description of Artificial Sequence Synthetic oligonucleotide
gaaactgtagattctccaagatcca

SEQ ID NO: 14 - DNA, 27 bp, Artificial Sequence
  Description of Artificial Sequence Synthetic primer
aacaccgggtcttaattcttatatcaa

SEQ ID NO: 15 - DNA, 28 bp, Artificial Sequence
  Description of Artificial Sequence Synthetic primer
ggtggtaaaattcccatagtagttcttt

SEQ ID NO: 16 - DNA, 19 bp, Artificial Sequence
  Description of Artificial Sequence Synthetic primer
cgagcctaacgttttgggc

SEQ ID NO: 17 - DNA, 21 bp, Artificial Sequence
  Description of Artificial Sequence Synthetic primer
gctccaggaattgttcgggta

SEQ ID NO: 18 - DNA, 28 bp, Artificial Sequence
  Description of Artificial Sequence Synthetic primer
ccctccatatcttagacaacatattgtg

SEQ ID NO: 19 - DNA, 22 bp, Artificial Sequence
  Description of Artificial Sequence Synthetic primer
cccaacactcctggttcacagc

SEQ ID NO: 20 - DNA, 26 bp, Artificial Sequence
  Description of Artificial Sequence Synthetic primer
actgcattytcatacttyctrgaatc

SEQ ID NO: 21 - DNA, 20 bp, Artificial Sequence
  Description of Artificial Sequence Synthetic primer
ccrggyttgaccagtgctgt

SEQ ID NO: 22 - DNA, 21 bp, Artificial Sequence
  Description of Artificial Sequence Synthetic primer
ggattgacctgtgcctgttgc

SEQ ID NO: 23 - DNA, 26 bp, Artificial Sequence
  Description of Artificial Sequence Synthetic primer
gcattagaaatgtcctcttttgctgc

SEQ ID NO: 24 - DNA, 24 bp, Artificial Sequence
  Description of Artificial Sequence Synthetic primer
agaaacacgtgccgcttacgccca

SEQ ID NO: 25 - DNA, 29 bp, Artificial Sequence
  Description of Artificial Sequence Synthetic primer
ccatttccyttyttraactcytcaaacca

SEQ ID NO: 26 - DNA, 22 bp, Artificial Sequence
  Description of Artificial Sequence Synthetic primer
ayacagcagcagttagcctcct

SEQ ID NO: 27 - DNA, 22 bp, Artificial Sequence
  Description of Artificial Sequence Synthetic primer
gctgccgtargtagtccctgtt

SEQ ID NO: 28 - DNA, 20 bp, Artificial Sequence
  Description of Artificial Sequence Synthetic primer
caggattgcagcagggaaga

SEQ ID NO: 29 - DNA, 21 bp, Artificial Sequence
  Description of Artificial Sequence Synthetic primer
atgatcaccaggytctacccc

SEQ ID NO: 30 - DNA, 17 bp, Artificial Sequence
  Description of Artificial Sequence Synthetic primer
gctgggagcgcggtatc

SEQ ID NO: 31 - DNA, 20 bp, Artificial Sequence
  Description of Artificial Sequence Synthetic primer
ggaagcccaatggtcctcat

SEQ ID NO: 32 - DNA, 20 bp, Artificial Sequence
  Description of Artificial Sequence Synthetic primer
tggaagcctggctgaaagag

SEQ ID NO: 33 - DNA, 20 bp, Artificial Sequence
  Description of Artificial Sequence Synthetic primer
tcatccccactgaccagcat

SEQ ID NO: 34 - DNA, 25 bp, Artificial Sequence
  Description of Artificial Sequence Synthetic probe
tttgttgctgttgcccggttcctac

SEQ ID NO: 35 - DNA, 25 bp, Artificial Sequence
  Description of Artificial Sequence Synthetic probe
agggttgcgctacagatccatgttg

SEQ ID NO: 36 - DNA, 25 bp, Artificial Sequence
  Description of Artificial Sequence Synthetic probe
aaaattttttgccatcagaaatttg

SEQ ID NO: 37 - DNA, 25 bp, Artificial Sequence
  Description of Artificial Sequence Synthetic probe
gctactaggcaatggcaggtttgtg

SEQ ID NO: 38 - DNA, 25 bp, Artificial Sequence
  Description of Artificial Sequence Synthetic probe
agggctccgatacagatctatgcta

SEQ ID NO: 39 - DNA, 25 bp, Artificial Sequence
  Description of Artificial Sequence Synthetic probe
cttgctgcttttacctggttcgtac

SEQ ID NO: 40 - DNA, 25 bp, Artificial Sequence
  Description of Artificial Sequence Synthetic probe
gggttgcgctataggtccatgttac

SEQ ID NO: 41 - DNA, 25 bp, Artificial Sequence
  Description of Artificial Sequence Synthetic probe
tttgctgctgttaccagggtcctac

SEQ ID NO: 42 - DNA, 25 bp, Artificial Sequence
  Description of Artificial Sequence Synthetic probe
taggtccatgttactgggcaacggt

SEQ ID NO: 43 - DNA, 25 bp, Artificial Sequence
  Description of Artificial Sequence Synthetic probe
tctgggcaacggccgttatgtgcct

SEQ ID NO: 44 - DNA, 25 bp, Artificial Sequence
  Description of Artificial Sequence Synthetic probe
cttctccctggctcctacacctatg

SEQ ID NO: 45 - DNA, 25 bp, Artificial Sequence
  Description of Artificial Sequence Synthetic probe
tctccctggctcctacacctatgag

SEQ ID NO: 46 - DNA, 25 bp, Artificial Sequence
  Description of Artificial Sequence Synthetic probe
ggcttgcgttaccgatccatgcttt

SEQ ID NO: 47 - DNA, 25 bp, Artificial Sequence
  Description of Artificial Sequence Synthetic probe
cctgctgcttctcccaggctcctac

SEQ ID NO: 48 - DNA, 25 bp, Artificial Sequence
  Description of Artificial Sequence Synthetic probe
gatccatgcttttgggtaacggacg

SEQ ID NO: 49 - DNA, 25 bp, Artificial Sequence
  Description of Artificial Sequence Synthetic probe
tggcttgcgttaccgatccatgctt

SEQ ID NO: 50 - DNA, 25 bp, Artificial Sequence
  Description of Artificial Sequence Synthetic probe
ccgtaacgctggcttgcgttaccga

SEQ ID NO: 51 - DNA, 25 bp, Artificial Sequence
  Description of Artificial Sequence Synthetic probe
cctgctgcttctcccaggctcctac

SEQ ID NO: 52 - DNA, 25 bp, Artificial Sequence
  Description of Artificial Sequence Synthetic probe
ccgtaacgctggcttgcgttaccga

SEQ ID NO: 53 - DNA, 25 bp, Artificial Sequence
  Description of Artificial Sequence Synthetic probe
ccgatccatgcttctgggtaacgga

SEQ ID NO: 54 - DNA, 25 bp, Artificial Sequence
  Description of Artificial Sequence Synthetic probe
cctgctgcttctcccaggctcctac

SEQ ID NO: 55 - DNA, 25 bp, Artificial Sequence
  Description of Artificial Sequence Synthetic probe
aaaattcttcgctgttaaaaacctg

SEQ ID NO: 56 - DNA, 25 bp, Artificial Sequence
  Description of Artificial Sequence Synthetic probe
tgcttctgggtaacggacgttatgt

SEQ ID NO: 57 - DNA, 25 bp, Artificial Sequence
  Description of Artificial Sequence Synthetic probe
cgctgttaaaaacctgctgcttctc

SEQ ID NO: 58 - DNA, 25 bp, Artificial Sequence
  Description of Artificial Sequence Synthetic probe
aacctgctgcttctacccggttctt

SEQ ID NO: 59 - DNA, 25 bp, Artificial Sequence
  Description of Artificial Sequence Synthetic probe
gctgcttctacccggttcttacacc

SEQ ID NO: 60 - DNA, 25 bp, Artificial Sequence
  Description of Artificial Sequence Synthetic probe
cctgctgcttctacccggttcttac

SEQ ID NO: 61 - DNA, 25 bp, Artificial Sequence
  Description of Artificial Sequence Synthetic probe
caggtccatgcttctgggaaatggt

SEQ ID NO: 62 - DNA, 25 bp, Artificial Sequence
  Description of Artificial Sequence Synthetic probe
gcgctacaggtccatgcttctggga

SEQ ID NO: 63 - DNA, 25 bp, Artificial Sequence
  Description of Artificial Sequence Synthetic probe
catgcttctgggaaatggtcgttat

SEQ ID NO: 64 - DNA, 25 bp, Artificial Sequence
  Description of Artificial Sequence Synthetic probe
caatgctggcctacgctaccggtcc

SEQ ID NO: 65 - DNA, 25 bp, Artificial Sequence
  Description of Artificial Sequence Synthetic probe
tggcctacgctaccggtccatgctt

SEQ ID NO: 66 - DNA, 25 bp, Artificial Sequence
  Description of Artificial Sequence Synthetic probe
tgctggcctacgctaccggtccatg

SEQ ID NO: 67 - DNA, 25 bp, Artificial Sequence
  Description of Artificial Sequence Synthetic probe
tttgccattaagaacctcctactct

SEQ ID NO: 68 - DNA, 25 bp, Artificial Sequence
  Description of Artificial Sequence Synthetic probe
cctcctactcttggcgggctcatac

SEQ ID NO: 69 - DNA, 25 bp, Artificial Sequence
  Description of Artificial Sequence Synthetic probe
tcctcagaagttttttgccattaag

SEQ ID NO: 70 - DNA, 25 bp, Artificial Sequence
  Description of Artificial Sequence Synthetic probe
ccgcaatgcgggcctccgttatcgc

SEQ ID NO: 71 - DNA, 25 bp, Artificial Sequence
  Description of Artificial Sequence Synthetic probe
cgctccatgttgttgggaaacggcc

SEQ ID NO: 72 - DNA, 25 bp, Artificial Sequence
  Description of Artificial Sequence Synthetic probe
ggcctccgttatcgctccatgttgt

SEQ ID NO: 73 - DNA, 25 bp, Artificial Sequence
  Description of Artificial Sequence Synthetic probe
aatgttgctgggcaatggtcgctat

SEQ ID NO: 74 - DNA, 25 bp, Artificial Sequence
  Description of Artificial Sequence Synthetic probe
cccttgactatatggacaacgtcaa

SEQ ID NO: 75 - DNA, 25 bp, Artificial Sequence
  Description of Artificial Sequence Synthetic probe
gcctcagaagttctttgccattaaa

SEQ ID NO: 76 - DNA, 25 bp, Artificial Sequence
  Description of Artificial Sequence Synthetic probe
gccccaaaagttttttgccattaaa

SEQ ID NO: 77 - DNA, 25 bp, Artificial Sequence
  Description of Artificial Sequence Synthetic probe
ttgttgggaaacggmcgctacgtgc

SEQ ID NO: 78 - DNA, 25 bp, Artificial Sequence
  Description of Artificial Sequence Synthetic probe
tgttgttgggaaacggmcgctacgt

SEQ ID NO: 79 - DNA, 25 bp, Artificial Sequence
  Description of Artificial Sequence Synthetic probe
cgcctccgtccgcttcgacagcgtc

SEQ ID NO: 80 - DNA, 25 bp, Artificial Sequence
  Description of Artificial Sequence Synthetic probe
ttcgacagcgtcaacctctacgcca

SEQ ID NO: 81 - DNA, 25 bp, Artificial Sequence
  Description of Artificial Sequence Synthetic probe
cgtccgcttcgacagcgtcaacctc

SEQ ID NO: 82 - DNA, 25 bp, Artificial Sequence
  Description of Artificial Sequence Synthetic probe
ccccatggacaacgtcaacccattc

SEQ ID NO: 83 - DNA, 25 bp, Artificial Sequence
  Description of Artificial Sequence Synthetic probe
cctgctcctgcttcccggctcctac

SEQ ID NO: 84 - DNA, 25 bp, Artificial Sequence
  Description of Artificial Sequence Synthetic probe
gttctttgccatcaagaacctgctc

SEQ ID NO: 85 - DNA, 25 bp, Artificial Sequence
  Description of Artificial Sequence Synthetic probe
tcaagaacctgctcctgcttcccgg

SEQ ID NO: 86 - DNA, 25 bp, Artificial Sequence
  Description of Artificial Sequence Synthetic probe
gttggaccccatggacaacgtcaac

SEQ ID NO: 87 - DNA, 25 bp, Artificial Sequence
  Description of Artificial Sequence Synthetic probe
tcgttggaccccatggacaacgtca

SEQ ID NO: 88 - DNA, 25 bp, Artificial Sequence
  Description of Artificial Sequence Synthetic probe
ttggaccccatggacaacgtcaacc

SEQ ID NO: 89 - DNA, 25 bp, Artificial Sequence
  Description of Artificial Sequence Synthetic probe
gccccaaaagttctttgccatcaag

SEQ ID NO: 90 - DNA, 25 bp, Artificial Sequence
  Description of Artificial Sequence Synthetic probe
ctttgccatcaagaacctgctcctg

SEQ ID NO: 91 - DNA, 25 bp, Artificial Sequence
  Description of Artificial Sequence Synthetic probe
gttctttgccatcaagaacctgctc

SEQ ID NO: 92 - DNA, 25 bp, Artificial Sequence
  Description of Artificial Sequence Synthetic probe
ccgctacgtgcccttccacatccaa

SEQ ID NO: 93 - DNA, 25 bp, Artificial Sequence
  Description of Artificial Sequence Synthetic probe
ttctttgccatcaagaacctgctcc

SEQ ID NO: 94 - DNA, 25 bp, Artificial Sequence
  Description of Artificial Sequence Synthetic probe
ccgctacgtgcccttccacatccaa

SEQ ID NO: 95 - DNA, 25 bp, Artificial Sequence
  Description of Artificial Sequence Synthetic probe
gctagaccccatggacaatgtcaac

SEQ ID NO: 96 - DNA, 25 bp, Artificial Sequence
  Description of Artificial Sequence Synthetic probe
gaacctgctcctgctccccggctct

SEQ ID NO: 97 - DNA, 25 bp, Artificial Sequence
  Description of Artificial Sequence Synthetic probe
tctgggcaatggccgctacgtgccc

SEQ ID NO: 98 - DNA, 25 bp, Artificial Sequence
  Description of Artificial Sequence Synthetic probe
ttctttgccatcaagaacctgctcc

SEQ ID NO: 99 - DNA, 25 bp, Artificial Sequence
  Description of Artificial Sequence Synthetic probe
gttctttgccatcaagaacctgctc

SEQ ID NO: 100 - DNA, 25 bp, Artificial Sequence
  Description of Artificial Sequence Synthetic probe
ctttgccatcaagaacctgctcctg

SEQ ID NO: 101 - DNA, 25 bp, Artificial Sequence
  Description of Artificial Sequence Synthetic probe
ctttgccatcaagaacctgctcctg

SEQ ID NO: 102 - DNA, 25 bp, Artificial Sequence
  Description of Artificial Sequence Synthetic probe
ccgctacgtgcccttccacatccaa

SEQ ID NO: 103 - DNA, 25 bp, Artificial Sequence
  Description of Artificial Sequence Synthetic probe
gttctttgccatcaagaacctgctc

SEQ ID NO: 104 - DNA, 25 bp, Artificial Sequence
  Description of Artificial Sequence Synthetic probe
cctgcttctgctccccggttcctac

SEQ ID NO: 105 - DNA, 25 bp, Artificial Sequence
  Description of Artificial Sequence Synthetic probe
caagaacctgcttctgctccccggt

SEQ ID NO: 106 - DNA, 25 bp, Artificial Sequence
  Description of Artificial Sequence Synthetic probe
ctgcttctgctccccggttcctaca

SEQ ID NO: 107 - DNA, 25 bp, Artificial Sequence
  Description of Artificial Sequence Synthetic probe
ccgctccatgttgctaggcaacggc

SEQ ID NO: 108 - DNA, 25 bp, Artificial Sequence
  Description of Artificial Sequence Synthetic probe
catcaagaacctgctcctactcccg

SEQ ID NO: 109 - DNA, 25 bp, Artificial Sequence
  Description of Artificial Sequence Synthetic probe
catgttgctaggcaacggccgctac

SEQ ID NO: 110 - DNA, 25 bp, Artificial Sequence
  Description of Artificial Sequence Synthetic probe
gaacctgctcctgcttcccggttcc

SEQ ID NO: 111 - DNA, 25 bp, Artificial Sequence
  Description of Artificial Sequence Synthetic probe
gctcctgcttcccggttcctacacc

SEQ ID NO: 112 - DNA, 25 bp, Artificial Sequence
  Description of Artificial Sequence Synthetic probe
gcttcccggttcctacacctacgag

SEQ ID NO: 113 - DNA, 25 bp, Artificial Sequence
  Description of Artificial Sequence Synthetic probe
ctccatgcttttgggcaatggccgc

SEQ ID NO: 114 - DNA, 25 bp, Artificial Sequence
  Description of Artificial Sequence Synthetic probe
tttgggcaatggccgctacgtgccc

SEQ ID NO: 115 - DNA, 25 bp, Artificial Sequence
  Description of Artificial Sequence Synthetic probe
gcttttgggcaatggccgctacgtg

SEQ ID NO: 116 - DNA, 25 bp, Artificial Sequence
  Description of Artificial Sequence Synthetic probe
tctgggcaacggccgctacgtaccc

SEQ ID NO: 117 - DNA, 25 bp, Artificial Sequence
  Description of Artificial Sequence Synthetic probe
ctttgccatcaagaacctgctcctg

SEQ ID NO: 118 - DNA, 25 bp, Artificial Sequence
  Description of Artificial Sequence Synthetic probe
ccgctacgtgcccttccacatacaa

SEQ ID NO: 119 - DNA, 25 bp, Artificial Sequence
  Description of Artificial Sequence Synthetic probe
gttctttgccatcaagaacctgctc

SEQ ID NO: 120 - DNA, 25 bp, Artificial Sequence
  Description of Artificial Sequence Synthetic probe
tagaccccatggacaatgtcaaccc

SEQ ID NO: 121 - DNA, 25 bp, Artificial Sequence
  Description of Artificial Sequence Synthetic probe
cgctagaccccatggacaatgtcaa

SEQ ID NO: 122 - DNA, 25 bp, Artificial Sequence
  Description of Artificial Sequence Synthetic probe
tctttgccatcaagaacctgctcct

SEQ ID NO: 123 - DNA, 25 bp, Artificial Sequence
  Description of Artificial Sequence Synthetic probe
ttccatgcttctgggcaacggccgc

SEQ ID NO: 124 - DNA, 25 bp, Artificial Sequence
  Description of Artificial Sequence Synthetic probe
tctttgccatcaagaacctgctcct

SEQ ID NO: 125 - DNA, 25 bp, Artificial Sequence
  Description of Artificial Sequence Synthetic probe
ccgctacgtgcccttccacatccaa

SEQ ID NO: 126 - DNA, 25 bp, Artificial Sequence
  Description of Artificial Sequence Synthetic probe
ccccatggacaacgtcaacccattc

SEQ ID NO: 127 - DNA, 25 bp, Artificial Sequence
  Description of Artificial Sequence Synthetic probe
tctttgccatcaagaacctgctcct

SEQ ID NO: 128 - DNA, 25 bp, Artificial Sequence
  Description of Artificial Sequence Synthetic probe
ttccatgcttctgggcaacggccgc

SEQ ID NO: 129 - DNA, 25 bp, Artificial Sequence
  Description of Artificial Sequence Synthetic probe
gcgctaccgttccatgttgttgggc

SEQ ID NO: 130 - DNA, 25 bp, Artificial Sequence
  Description of Artificial Sequence Synthetic probe
gctggatcccatggacaatgtaaac

SEQ ID NO: 131 - DNA, 25 bp, Artificial Sequence
  Description of Artificial Sequence Synthetic probe
ttccatgttgttgggcaacggtcgc

SEQ ID NO: 132 - DNA, 25 bp, Artificial Sequence
  Description of Artificial Sequence Synthetic probe
ccgttccatgctgttgggcaacggc

SEQ ID NO: 133 - DNA, 25 bp, Artificial Sequence
  Description of Artificial Sequence Synthetic probe
ttccatgctgttgggcaacggccgc

SEQ ID NO: 134 - DNA, 25 bp, Artificial Sequence
  Description of Artificial Sequence Synthetic probe
ccgttccatgctcctgggcaacggc

SEQ ID NO: 135 - DNA, 25 bp, Artificial Sequence
  Description of Artificial Sequence Synthetic probe
ctttgccatcaagaacctgctcctg

SEQ ID NO: 136 - DNA, 25 bp, Artificial Sequence
  Description of Artificial Sequence Synthetic probe
gttctttgccatcaagaacctgctc

SEQ ID NO: 137 - DNA, 25 bp, Artificial Sequence
  Description of Artificial Sequence Synthetic probe
gttctttgccatcaagaacctgctc

SEQ ID NO: 138 - DNA, 25 bp, Artificial Sequence
  Description of Artificial Sequence Synthetic probe
ccccatggacaacgtcaacccattc

SEQ ID NO: 139 - DNA, 25 bp, Artificial Sequence
  Description of Artificial Sequence Synthetic probe
gcgctacgtgcccttccatattcaa

SEQ ID NO: 140 - DNA, 25 bp, Artificial Sequence
  Description of Artificial Sequence Synthetic probe
cgggcgctacgtgcccttccatatt

SEQ ID NO: 141 - DNA, 25 bp, Artificial Sequence
  Description of Artificial Sequence Synthetic probe
ttttgccattaagaacctgcttctg

SEQ ID NO: 142 - DNA, 25 bp, Artificial Sequence
  Description of Artificial Sequence Synthetic probe
gcgctacgtgccattccacatycag

SEQ ID NO: 143 - DNA, 25 bp, Artificial Sequence
  Description of Artificial Sequence Synthetic probe
gctggaccccatggacaacgtaaat

SEQ ID NO: 144 - DNA, 25 bp, Artificial Sequence
  Description of Artificial Sequence Synthetic probe
atttttcgccattaaaaatctcctg

SEQ ID NO: 145 - DNA, 25 bp, Artificial Sequence
  Description of Artificial Sequence Synthetic probe
tggtctgcgctaccgttctatgctc

SEQ ID NO: 146 - DNA, 25 bp, Artificial Sequence
  Description of Artificial Sequence Synthetic probe
cgccattaaaaatctcctgctcctg

SEQ ID NO: 147 - DNA, 25 bp, Artificial Sequence
  Description of Artificial Sequence Synthetic probe
cagatcccatggacaatgttaaccc

SEQ ID NO: 148 - DNA, 25 bp, Artificial Sequence
  Description of Artificial Sequence Synthetic probe
cttgggcaacgggcgttacgtaccc

SEQ ID NO: 149 - DNA, 25 bp, Artificial Sequence
  Description of Artificial Sequence Synthetic probe
gctcttgggcaacgggcgttacgta

(19) United States
(12) Patent Application Publication
(10) Pub. No.: US 2009/0105092 A1
(43) Pub. Date: Apr. 23, 2009

(54) VIRAL DATABASE METHODS

(75) Inventors: W. Ian LIPKIN, New York, NY (US); Gustavo PALACIOS, New York, NY (US); Thomas BRIESE, White Plains, NY (US); Omar JABADO, New York, NY (US)

Correspondence Address: WilmerHale/Columbia University, 399 PARK AVENUE, NEW YORK, NY, 10022, US

(73) Assignee: THE TRUSTEES OF COLUMBIA UNIVERSITY IN THE CITY OF NEW YORK, New York, NY (US)

(21) Appl. No.: 11/945,938

(22) Filed: Nov. 27, 2007

Related U.S. Application Data
(60) Provisional application No. 60/861,365, filed on Nov. 28, 2006.

(51) Int. Cl. C40B 40/06; G06F 17/30; C12Q 1/70

(52) U.S. Cl. 506/16; 707/102; 707/200; 435/5

(57) ABSTRACT

Disclosed are methods for designing oligonucleotides that can detect/identify any unknown or known virus of a particular taxon. Also provided are methods to establish, implement and validate bioinformatics tools and databases to support microarray design. The invention also provides specialized arrays for detection and speciation of select viral agents and viruses as well as a set of oligonucleotides that can detect/identify any unknown or known virus of a particular taxon.

This application claims the benefit of and priority to U.S. provisional patent application Ser. No. 60/861,365, filed Nov. 28, 2006, and the CD-ROM sequence appendices filed with that application (and listed below), the disclosure of all of which is hereby incorporated by reference in its entirety for all purposes.

The invention disclosed herein was made with Government support under NIH Grant No. A151992, A1056118, and A155466 from the Department of Health and Human Services. Accordingly, the U.S. Government has certain rights in this invention. Accordingly, the U.S. Government has certain rights in this invention.

All patents, patent applications and publications cited herein are hereby incorporated by reference in their entirety. The disclosures of these publications in their entireties are hereby incorporated by reference into this application in order to more fully describe the state of the art as known to those skilled therein as of the date of the invention described and claimed herein.

Fourteen (14) compact discs (CDs) filed as part of U.S. Ser. No. 60/861,365 on Nov. 28, 2006 are hereby incorporated by reference in their entirety. The names of the files on the CDs are as follows:

| | | | | | File size | |
|---|---|---|---|---|---|---|
| | | Machine | Operating | File size in | on CD in | Date |
| CD Name | Name of file | Format | System | bytes | bytes | created |
| IR1900DB.1 | IR1900DB.1_Adenoviridae.txt | IBM-PC | MS-Windows | 1,081,199 | 1,081,344 | Nov. 20, 2006 |
| | IR1900DB.1_ADV_data.txt | IBM-PC | MS-Windows | 2,210,805 | 2,210,840 | Nov. 20, 2006 |
| | IR1900DB.1_Bunyaviridae.txt | IBM-PC | MS-Windows | 1,668,869 | 1,669,120 | Nov. 20, 2006 |
| | IR1900DB.1_BV_data.txt | IBM-PC | MS-Windows | 4,482,413 | 4,483,072 | Nov. 20, 2006 |
| | IR1900DB.1_Herpesviridae.txt | IBM-PC | MS-Windows | 1,915,160 | 1,915,928 | Nov. 20, 2006 |
| | IR1900DB.1_HPV_data.txt | IBM-PC | MS-Windows | 4,345,248 | 4,345,856 | Nov. 20, 2006 |

TABLE 1

| | | | | | File size | |
|---|---|---|---|---|---|---|
| | | Machine | Operating | File size in | on CD in | Date |
| CD Name | Name of file | Format | System | bytes | bytes | created |
| (ADENOVIRIDAE | TABLE 1 (ADENOVIRIDAE TO | IBM-PC | MS-Windows | 469,892 | 471,040 | Nov. 20, 2006 |
| TO | BUNYAVIRIDAE)_arenav_data.txt | | | | | |
| BUNYAVIRIDAE) | TABLE 1 (ADENOVIRIDAE TO | IBM-PC | MS-Windows | 22,166 | 22,528 | Nov. 20, 2006 |
| | BUNYAVIRIDAE)_Arteriviridae.txt | | | | | |
| | TABLE 1 (ADENOVIRIDAE TO | IBM-PC | MS-Windows | 69,770 | 71,680 | Nov. 20, 2006 |
| | BUNYAVIRIDAE)_arterv_data.txt | | | | | |
| | TABLE 1 (ADENOVIRIDAE TO | IBM-PC | MS-Windows | 13,510 | 14,336 | Nov. 20, 2006 |
| | BUNYAVIRIDAE)_asfarv_data.txt | | | | | |
| | TABLE 1 (ADENOVIRIDAE TO | IBM-PC | MS-Windows | 3,843 | 4,096 | Nov. 20, 2006 |
| | BUNYAVIRIDAE)_Asfarviridae.txt | | | | | |
| | TABLE 1 (ADENOVIRIDAE TO | IBM-PC | MS-Windows | 126,414 | 126,976 | Nov. 20, 2006 |
| | BUNYAVIRIDAE)_astrov_data.txt | | | | | |
| | TABLE 1 (ADENOVIRIDAE TO | IBM-PC | MS-Windows | 23,658 | 24,576 | Nov. 20, 2006 |
| | BUNYAVIRIDAE)_Astroviridae.txt | | | | | |
| | TABLE 1 (ADENOVIRIDAE TO | IBM-PC | MS-Windows | 6,071 | 6,144 | Nov. 20, 2006 |
| | BUNYAVIRIDAE)_Birnaviridae.txt | | | | | |
| | TABLE 1 (ADENOVIRIDAE TO | IBM-PC | MS-Windows | 23,676 | 24,576 | Nov. 20, 2006 |
| | BUNYAVIRIDAE)_Birnaviridae_data.txt | | | | | |
| | TABLE 1 (ADENOVIRIDAE TO | IBM-PC | MS-Windows | 1,987 | 2,048 | Nov. 20, 2006 |
| | BUNYAVIRIDAE)_Bornaviridae.txt | | | | | |
| | TABLE 1 (ADENOVIRIDAE TO | IBM-PC | MS-Windows | 6,112 | 6,144 | Nov. 20, 2006 |
| | BUNYAVIRIDAE)_Bornaviridae_data.txt | | | | | |
| | TABLE 1 (ADENOVIRIDAE TO | IBM-PC | MS-Windows | 1,668,869 | 1,669,120 | Nov. 20, 2006 |
| | BUNYAVIRIDAE)_Bunyaviridae.txt | | | | | |
| | TABLE 1 (ADENOVIRIDAE TO | IBM-PC | MS-Windows | 4,482,413 | 4,483,072 | Nov. 20, 2006 |
| | BUNYAVIRIDAE)_BV_data.txt | | | | | |
| TABLE 1 | TABLE 1 TEXT TAB | IBM-PC | MS-Windows | 1,081,199 | 1,081,344 | Nov. 20, 2006 |
| TEXT TAB | DELIMITED.txt | | | | | |
| DELIMITED | | | | | | |

TABLE 2

| | | | | | File size | |
|---|---|---|---|---|---|---|
| | | Machine | Operating | File size in | on CD in | Date |
| CD Name | Name of file | Format | System | bytes | bytes | created |
| (CALICIVIRIDAE | TABLE 2 (CALICIVIRIDAE TO | IBM-PC | MS-Windows | 1,519,973 | 1,521,664 | Nov. 20, 2006 |
| TO | HEPATITIS-E- | | | | | |
| HEPATITIS- | LIKE)_Caliciviridae.TXT | | | | | |
| E-LIKE) | TABLE 2 (CALICIVIRIDAE TO | IBM-PC | MS-Windows | 7,386,354 | 7,387,136 | Nov. 20, 2006 |
| | HEPATITIS-E- | | | | | |
| | LIKE)_Caliciviridae_data.TXT | | | | | |
| | TABLE 2 (CALICIVIRIDAE TO | IBM-PC | MS-Windows | 64,397 | 65,536 | Nov. 20, 2006 |
| | HEPATITIS-E- | | | | | |
| | LIKE)_Circoviridae.TXT | | | | | |
| | TABLE 2 (CALICIVIRIDAE TO | IBM-PC | MS-Windows | 173,587 | 174,080 | Nov. 20, 2006 |
| | HEPATITIS-E- | | | | | |
| | LIKE)_Circoviridae_data.TXT | | | | | |
| | TABLE 2 (CALICIVIRIDAE TO | IBM-PC | MS-Windows | 206,568 | 206,848 | Nov. 20, 2006 |
| | HEPATITIS-E- | | | | | |
| | LIKE)_Coronaviridae.TXT | | | | | |
| | TABLE 2 (CALICIVIRIDAE TO | IBM-PC | MS-Windows | 1,180,446 | 1,181,696 | Nov. 20, 2006 |
| | HEPATITIS-E- | | | | | |
| | LIKE)_Coronaviridae_data.TXT | | | | | |
| | TABLE 2 (CALICIVIRIDAE TO | IBM-PC | MS-Windows | 185,127 | 186,368 | Nov. 20, 2006 |
| | HEPATITIS-E- | | | | | |
| | LIKE)_Deltavirus.TXT | | | | | |
| | TABLE 2 (CALICIVIRIDAE TO | IBM-PC | MS-Windows | 489,561 | 491,520 | Nov. 20, 2006 |
| | HEPATITIS-E- | | | | | |
| | LIKE)_Deltavirus_data.TXT | | | | | |
| | TABLE 2 (CALICIVIRIDAE TO | IBM-PC | MS-Windows | 753 | 2,048 | Nov. 20, 2006 |
| | HEPATITIS-E-LIKE)_families.txt | | | | | |
| | TABLE 2 (CALICIVIRIDAE TO | IBM-PC | MS-Windows | 22,165 | 22,528 | Nov. 20, 2006 |
| | HEPATITIS-E- | | | | | |
| | LIKE)_Filoviridae.TXT | | | | | |
| | TABLE 2 (CALICIVIRIDAE TO | IBM-PC | MS-Windows | 99,611 | 100,352 | Nov. 20, 2006 |
| | HEPATITIS-E- | | | | | |
| | LIKE)_Filoviridae_data.TXT | | | | | |
| | TABLE 2 (CALICIVIRIDAE TO | IBM-PC | MS-Windows | 2,387,693 | 2,387,968 | Nov. 20, 2006 |
| | HEPATITIS-E- | | | | | |
| | LIKE)_Flaviviridae.TXT | | | | | |
| | TABLE 2 (CALICIVIRIDAE TO | IBM-PC | MS-Windows | 8,717,760 | 8,718,336 | Nov. 20, 2006 |
| | HEPATITIS-E- | | | | | |
| | LIKE)_Flaviviridae_data.TXT | | | | | |
| | TABLE 2 (CALICIVIRIDAE TO | IBM-PC | MS-Windows | 205,593 | 206,848 | Nov. 20, 2006 |
| | HEPATITIS-E- | | | | | |
| | LIKE)_Hepadnaviridae.TXT | | | | | |
| | TABLE 2 (CALICIVIRIDAE TO | IBM-PC | MS-Windows | 1,092,676 | 1,093,632 | Nov. 20, 2006 |
| | HEPATITIS-E- | | | | | |
| | LIKE)_Hepadnaviridae_data.TXT | | | | | |
| | TABLE 2 (CALICIVIRIDAE TO | IBM-PC | MS-Windows | 207,587 | 208,896 | Nov. 20, 2006 |
| | HEPATITIS-E-LIKE)_Hepatitis E- | | | | | |
| | like viruses.TXT | | | | | |
| | TABLE 2 (CALICIVIRIDAE TO | IBM-PC | MS-Windows | 1,402,040 | 1,402,880 | Nov. 20, 2006 |
| | HEPATITIS-E-LIKE)_Hepatitis E- | | | | | |
| | like viruses_Data.TXT | | | | | |

TABLE 3

| | | | | | File size | |
|---|---|---|---|---|---|---|
| | | Machine | Operating | File size in | on CD in | Date |
| CD Name | Name of file | Format | System | bytes | bytes | created |
| (HERPES- | TABLE 3 (HERPESVIRIDAE TO | IBM-PC | MS-Windows | 753 | 2,048 | Nov. 20, 2006 |
| VIRIDAE | PARAMYXOVIRIDAE)_families.txt | | | | | |
| TO | TABLE 3 (HERPESVIRIDAE TO | IBM-PC | MS-Windows | 1,921,680 | 1,923,072 | Nov. 20, 2006 |
| PARAMYX- | PARAMYXOVIRIDAE)_Herpesviridae.txt | | | | | |
| OVIRIDAE) | TABLE 3 (HERPESVIRIDAE TO | IBM-PC | MS-Windows | 4,345,248 | 4,345,856 | Nov. 20, 2006 |
| | PARAMYXOVIRIDAE)_Herpesviridae_data.txt | | | | | |
| | TABLE 3 (HERPESVIRIDAE TO | IBM-PC | MS-Windows | 77,305 | 77,824 | Nov. 20, 2006 |
| | PARAMYXOVIRIDAE)_Iridoviridae.txt | | | | | |
| | TABLE 3 (HERPESVIRIDAE TO | IBM-PC | MS-Windows | 127,426 | 129,024 | Nov. 20, 2006 |
| | PARAMYXOVIRIDAE)_Iridoviridae_data.txt | | | | | |
| | TABLE 3 (HERPESVIRIDAE TO | IBM-PC | MS-Windows | 9,746 | 10,240 | Nov. 20, 2006 |
| | PARAMYXOVIRIDAE)_Iaryngotracheitis- | | | | | |
| | like viruses.txt | | | | | |
| | TABLE 3 (HERPESVIRIDAE TO | IBM-PC | MS-Windows | 15,674 | 16,384 | Nov. 20, 2006 |
| | PARAMYXOVIRIDAE)_Iaryngotracheitis- | | | | | |
| | like_Data.txt | | | | | |
| | TABLE 3 (HERPESVIRIDAE TO | IBM-PC | MS-Windows | 96,464 | 98,304 | Nov. 20, 2006 |
| | PARAMYXOVIRIDAE)_Nodaviridae.txt | | | | | |
| | TABLE 3 (HERPESVIRIDAE TO | IBM-PC | MS-Windows | 159,099 | 159,744 | Nov. 20, 2006 |
| | PARAMYXOVIRIDAE)_Nodaviridae_data.txt | | | | | |
| | TABLE 3 (HERPESVIRIDAE TO | IBM-PC | MS-Windows | 35,818 | 36,864 | Nov. 20, 2006 |
| | PARAMYXOVIRIDAE)_Orthomyxoviridae.txt | | | | | |
| | TABLE 3 (HERPESVIRIDAE TO | IBM-PC | MS-Windows | 68,192 | 69,632 | Nov. 20, 2006 |
| | PARAMYXOVIRIDAE)_Orthomyxoviridae_data.txt | | | | | |
| | TABLE 3 (HERPESVIRIDAE TO | IBM-PC | MS-Windows | 1,705,954 | 1,705,984 | Nov. 20, 2006 |
| | PARAMYXOVIRIDAE)_Papillomaviridae.txt | | | | | |
| | TABLE 3 (HERPESVIRIDAE TO | IBM-PC | MS-Windows | 2,939,371 | 2,940,928 | Nov. 20, 2006 |
| | PARAMYXOVIRIDAE)_Papillomaviridae_data.txt | | | | | |
| | TABLE 3 (HERPESVIRIDAE TO | IBM-PC | MS-Windows | 843,479 | 843,776 | Nov. 20, 2006 |
| | PARAMYXOVIRIDAE)_Paramyxoviridae.txt | | | | | |
| | TABLE 3 (HERPESVIRIDAE TO | IBM-PC | MS-Windows | 4,062,816 | 4,063,232 | Nov. 20, 2006 |
| | PARAMYXOVIRIDAE)_Paramyxoviridae_data.txt | | | | | |

TABLE 4

| | | | | | File size | |
|---|---|---|---|---|---|---|
| | | Machine | Operating | File size in | on CD in | Date |
| CD Name | Name of file | Format | System | bytes | bytes | created |
| (PARVO | TABLE 4 (PARVO AND | IBM-PC | MS-Windows | 753 | 2,048 | Nov. 20, 2006 |
| AND | PICORNA)_families.txt | | | | | |
| PICORNA) | TABLE 4 (PARVO AND | IBM-PC | MS-Windows | 304,272 | 305,152 | Nov. 20, 2006 |
| | PICORNA)_Parvoviridae.txt | | | | | |
| | TABLE 4 (PARVO AND | IBM-PC | MS-Windows | 976,408 | 976,896 | Nov. 20, 2006 |
| | PICORNA)_Parvoviridae_data.txt | | | | | |
| | TABLE 4 (PARVO AND | IBM-PC | MS-Windows | 3,884,679 | 3,885,056 | Nov. 20, 2006 |
| | PICORNA)_Picornaviridae.txt | | | | | |
| | TABLE 4 (PARVO AND | IBM-PC | MS-Windows | 12,225,777 | 12,226,560 | Nov. 20, 2006 |
| | PICORNA)_Picornaviridae_data.txt | | | | | |

TABLE 5

| | | | | | File size | |
|---|---|---|---|---|---|---|
| | | Machine | Operating | File size in | on CD in | Date |
| CD Name | Name of file | Format | System | bytes | bytes | created |
| (POLYOMA | TABLE 5 (POLYOMA TO | IBM-PC | MS-Windows | 753 | 2,048 | Nov. 20, 2006 |
| TO TOGA) | TOGA)_families.txt | | | | | |
| | TABLE 5 (POLYOMA TO | IBM-PC | MS-Windows | 87,829 | 88,064 | Nov. 20, 2006 |
| | TOGA)_Polyomaviridae.txt | | | | | |
| | TABLE 5 (POLYOMA TO | IBM-PC | MS-Windows | 362,827 | 364,544 | Nov. 20, 2006 |
| | TOGA)_Polyomaviridae_data.txt | | | | | |
| | TABLE 5 (POLYOMA TO | IBM-PC | MS-Windows | 519,214 | 520,192 | Nov. 20, 2006 |
| | TOGA)_Poxviridae.txt | | | | | |
| | TABLE 5 (POLYOMA TO | IBM-PC | MS-Windows | 1,276,662 | 1,277,952 | Nov. 20, 2006 |
| | TOGA)_Poxviridae_data.txt | | | | | |
| | TABLE 5 (POLYOMA TO | IBM-PC | MS-Windows | 1,051,864 | 1,052,672 | Nov. 20, 2006 |
| | TOGA)_Reoviridae.txt | | | | | |
| | TABLE 5 (POLYOMA TO | IBM-PC | MS-Windows | 5,215,090 | 5,216,256 | Nov. 20, 2006 |
| | TOGA)_Reoviridae_data.txt | | | | | |
| | TABLE 5 (POLYOMA TO | IBM-PC | MS-Windows | 1,547,365 | 1,548,288 | Nov. 20, 2006 |
| | TOGA)_Retroviridae.txt | | | | | |
| | TABLE 5 (POLYOMA TO | IBM-PC | MS-Windows | 5,872,327 | 5,873,664 | Nov. 20, 2006 |
| | TOGA)_Retroviridae_data.txt | | | | | |
| | TABLE 5 (POLYOMA TO | IBM-PC | MS-Windows | 342,600 | 344,064 | Nov. 20, 2006 |
| | TOGA)_Rhabdoviridae.txt | | | | | |
| | TABLE 5 (POLYOMA TO | IBM-PC | MS-Windows | 1,119,112 | 1,120,256 | Nov. 20, 2006 |
| | TOGA)_Rhabdoviridae_data.txt | | | | | |
| | TABLE 5 (POLYOMA TO | IBM-PC | MS-Windows | 767,840 | 768,000 | Nov. 20, 2006 |
| | TOGA)_Togaviridae.txt | | | | | |
| | TABLE 5 (POLYOMA TO | IBM-PC | MS-Windows | 2,523,183 | 2,525,184 | Nov. 20, 2006 |
| | TOGA)_Togaviridae_data.txt | | | | | |

The compact discs are referred to herein as “CD-ROM Table Appendix”, “CD-ROM and “CD-ROM Program Listing Appendix”, contain Tables 1-5. The CD-ROM Table Appendix contains tables of information relating to vertebrate virus amino acid and nucleic acid sequences representative of various taxa. Each table contains information about a different vertebrate virus family and is present as a separate plain text (ASCII) file on the compact disc. On the compact disc, these table files are entitled: Adenoviridae, Adenoviridae Data, Arenaviridae, Arenaviridae Data, Arteriviridae, Arteriviridae Data, Asfarviridae, Asfarviridae Data, Astroviridae, Astroviridae Data, Birnaviridae, Birnaviridae Data, Bornaviridae, Bornaviridae Data, Bunyaviridae, Bunyaviridae Data, Caliciviridae, Caliciviridae Data, Circoviridae, Circoviridae Data, Coronaviridae, Coronaviridae Data, Deltavirus, Deltavirus Data, Filoviridae, Filoviridae Data, Flaviviridae, Flaviviridae Data, Hepadnaviridae, Hepadnaviridae Data, Hepatitis E-like viruses, Hepatitis E-like viruses Data, Herpesviridae, Herpesviridae Data, Infectious laryngotrachetitis-like viruses, Infectious laryngotrachetitis-like viruses Data, Iridoviridae, Iridoviridae Data, Nodaviridae, Nodaviridae Data, Orthomyxoviridae, Orthomyxoviridae Data, Papillomaviridae, Papillomaviridae Data, Paramyxoviridae, Paramyxoviridae Data, Parvoviridae, Parvoviridae Data, Picornaviridae, Picornaviridae Data, Polyomaviridae, Polyomaviridae Data, Poxyiridae, Poxyiridae Data, Reoviridae, Reoviridae Data, Retroviridae, Retroviridae Data, Rhabdoviridae, Rhabdoviridae Data, Togaviridae, and Togaviridae Data.

A portion of the disclosure of this patent document contains material that is subject to copyright protection, including the CD-ROM Appendices. The copyright owner has no objection to the facsimile reproduction by anyone of the patent document or the patent disclosure, as it appears in the Patent and Trademark Office patent file or records, but otherwise reserves all copyright rights whatsoever.

### BACKGROUND

The effective control of contagion and clinical management of infectious diseases require tools that can provide rapid, accurate, and differential analysis. Genetic methods for detecting unknown microbial pathogens in clinical or environmental samples require the ability to overcome the high mutation rate of such pathogens. To overcome the high mutation rate and the high degree of microbial speciation, molecular probes that are based on conserved sequences are needed. However, such probes have not been generated due to: (1) lack of comprehensive and accurately curated microbial databases; (2) lack of thorough sequence analysis of such databases; and (3) lack of new methods to analyze such databases to provide collections or sets of molecular probes that can detect a wide range of pathogens in an environmental or clinical sample.

### SUMMARY OF THE INVENTION

The invention provides for methods to design oligonucleotides that can detect/identify any unknown or known virus of a particular taxon. The invention provides methods to establish, implement and validate bioinformatics tools and databases to support microarray design and updates. The invention provides for the design and implementation of software for extracting viral sequence updates, for maintaining and curating an updated, integrated ICTV/NCBI virus database, for validating the database by testing performance of microarrays using a random sample of viral targets, and for developing an array analysis software for automated evaluation of hybridization results.

The invention further provides for specialized arrays for detection and speciation of select viral agents and viruses. The invention provides for the design of oligonucleotide targets, the printing of sub-arrays, obtaining nucleic acid extracts from infected, cultured cells, and optimizing assay performance using infected culture extracts.

The invention further provides for array protocols and methods for use with clinical materials. The invention provides for optimizing methods for detection of viral select agents in physiologically relevant compartments including blood, urine, sputum, feces, and tissues and obtaining and analyzing nucleic acid extracts from infected animals or humans.

The invention provides methods to establish stable and sensitive viral microarray assays for public health laboratory, hospital-based clinical laboratory, and point-of-care use to enable differential diagnosis of infection by select priority agents, agents that may cause signs and symptoms that mimic those due to infection with select agents, and influenza viruses. Features that enhance the probability of the programmatic success include but are not limited to: (1) expertise in clinical microbiology, bioinformatics, and molecular diagnostics; (2) a sensitive and stable microarray platform; (3) access to databases of select agent and virus sequences; (4) a comprehensive inventory of extracts from infected, cultured cells; naturally and experimentally infected animals; and human victims of infection with select viral agents or viruses; (5) partnership in a dedicated, international surveillance network wherein clinical samples can be shared for assay optimization, validation, and implementation to support global public health; and/or (6) commercial partners with expertise in manufacture, licensure, and distribution of diagnostic reagents.

In one aspect, the invention provides methods or systems for identifying oligonucleotide sequences that hybridize to related but not necessarily identical sequence targets. The methods can provide an automated system for consensus primer and target design. Such methods or systems can be used for designing, for example, primers for consensus PCR and targets (i.e., oligonucleotides) for DNA microarrays.

In one aspect, the invention provides methods to identify sequences that discriminate taxa rather than genes. The invention allows for the design of degenerate primers and targets. It also facilitates detection and discovery of similar but not identical sequences as well as enhancing assay efficiency by reducing the number of primers and targets required to address a specific diagnostic challenge. In another aspect, the invention provides methods that comprise an application of Shannon's Entropy to molecular biology based diagnostics.

In one aspect, the invention provides a set of oligonucleotides for detecting vertebrate viruses, the set of oligonucleotides comprising a plurality of nucleic acid sequences that are reverse translated from at least about 10,000, about 20,000, about 30,000, about 40,000 or about 50,000 different amino acid sequences, each amino acid sequence comprising a motif conserved in a virus family (or a motif conserved in a genus or species of the virus family), wherein the virus family is selected from the group consisting of: Adenoviridae, Arenaviridae, Arteriviridae, Asfarviridae, Astroviridae, Birnaviridae, Bornaviridae, Bunyaviridae, Caliciviridae, Circoviridae, Coronaviridae, Deltavirus, Filoviridae, Flaviviridae, Hepadnaviridae, Hepatitis E-like viruses, Herpesviridae, Infectious laryngotrachetitis-like viruses, Iridoviridae, Nodaviridae, Orthomyxoviridae, Papillomaviridae, Paramyxoviridae, Parvoviridae, Picornaviridae, Polyomaviridae, Poxyiridae, Reoviridae, Retroviridae, Rhabdoviridae, and Togaviridae. The genera and species of these virus families are listed in the CD-ROM Appendix and are also known to those skilled in the art of virology. In another aspect, the plurality of nucleic acid sequences are reverse translated from at least 100 different amino acid sequences, wherein the amino acid sequences comprise at least 5 motifs conserved in a genus or species of each of the vertebrate virus families. In one aspect, the plurality of nucleic acid sequences is reverse translated from less than 5,000; 4,000; 3,000; 2,000; 1000; 950; 900; 850; 800; 750; 700; or 500 different amino acid sequences. In one aspect, the set of oligonucleotides is capable of hybridizing to a genome of any vertebrate virus. In one aspect, the amino acid sequences are selected from the amino acid sequences listed in the CD-ROM Table Appendix.

In one aspect, the invention provides set of oligonucleotides for detecting vertebrate viruses, the set of oligonucleotides comprising a plurality of nucleic acid sequences that are reverse translated from at least about 10,000, about 20,000, about 30,000, about 40,000 or about 50,000 different amino acid sequences, each amino acid sequence comprising a motif conserved in a different virus genus, wherein the virus genus is selected from the group consisting of: Asfivirus, Orthopoxvirus, Parapoxvirus, Avipoxvirus, Capripoxvirus, Leporipoxvirus, Suipoxvirus, Molluscipoxvirus, Yatapoxvirus, Entomopoxvirus A, Entomopoxvirus B, Entomopoxvirus C, Iridovirus, Chloriridovirus, Ranavirus, Lymphocystivirus, Simplexvirus, Varicellovirus, Cytomegalovirus, Muromegalovirus, Roseolovirus, Lymphocryptovirus, Rhadinovirus, Ichnovirus, Bracovirus, Polyomavirus, Papillomavirus, Mastadenovirus, Aviadenovirus, Orthoreovirus, Orbivirus, Rotavirus, Coltivirus, Aquareovirus, Cypovirus, Fijivirus, Phytoreovirus, Oryzavirus, Aquabimavirus, Avibimavirus, Entomobirnavirus, Influenzavirus A, Influenzavirus B, Influenzavirus C, Influenzavirus D, Paramyxovirus, Morbillivirus, Rubulavirus, Pneumovirus, Bornavirus, Marburgvirus, Ebolavirus, Arenavirus, Alpharetrovirus, Betaretrovirus, Gammaretrovirus, Type D Retrovirus group, Deltaretrovirus, Epsilonretrovirus, Lentivirus, Spumavirus, Bunyavirus, Hantavirus, Nairovirus, Phlebovirus, Tospovirus, Calicivirus, Enterovirus, Rhinovirus, Hepatovirus, Cardiovirus, Aphthovirus, Astrovirus, Flavivirus, Pestivirus, Hepacivirus, Alphanodavirus, Coronavirus, Torovirus, Alphavirus, Arterivirus, and Deltavirus. In one aspect, the amino acid sequences are selected from the amino acid sequences listed in the CD-ROM Table Appendix.

In one aspect, the invention provides a set of oligonucleotides for detecting vertebrate viruses, the set of oligonucleotides comprising a plurality of nucleic acid sequences that are reverse translated amino acid sequences, each amino acid sequence comprising a motif conserved in either: (a) a virus family, wherein the virus family is selected from the group consisting of: Adenoviridae, Arenaviridae, Arteriviridae, Asfarviridae, Astroviridae, Birnaviridae, Bornaviridae, Bunyaviridae, Caliciviridae, Circoviridae, Coronaviridae, Deltavirus, Filoviridae, Flaviviridae, Hepadnaviridae, Hepatitis E-like viruses, Herpesviridae, Infectious laryngotrachetitis-like viruses, Iridoviridae, Nodaviridae, Orthomyxoviridae, Papillomaviridae, Paramyxoviridae, Parvoviridae, Picornaviridae, Polyomaviridae, Poxyiridae, Reoviridae, Retroviridae, Rhabdoviridae, and Togaviridae; (b) a virus genus, wherein the virus genus is selected from the group consisting of: Asfivirus, Orthopoxvirus, Parapoxvirus, Avipoxvirus, Capripoxvirus, Leporipoxvirus, Suipoxvirus, Molluscipoxvirus, Yatapoxvirus, Entomopoxvirus A, Entomopoxvirus B, Entomopoxvirus C, Iridovirus, Chloriridovirus, Ranavirus, Lymphocystivirus, Simplexvirus, Varicellovirus, Cytomegalovirus, Muromegalovirus, Roseolovirus, Lymphocryptovirus, Rhadinovirus, Ichnovirus, Bracovirus, Polyomavirus, Papillomavirus, Mastadenovirus, Aviadenovirus, Orthoreovirus, Orbivirus, Rotavirus, Coltivirus, Aquareovirus, Cypovirus, Fijivirus, Phytoreovirus, Oryzavirus, Aquabirnavirus, Avibirnavirus, Entomobirnavirus, Influenzavirus A, Influenzavirus B, Influenzavirus C, Influenzavirus D, Paramyxovirus, Morbillivirus, Rubulavirus, Pneumovirus, Bornavirus, Marburgvirus, Ebolavirus, Arenavirus, Alpharetrovirus, Betaretrovirus, Gammaretrovirus, Type D Retrovirus group, Deltaretrovirus, Epsilonretrovirus, Lentivirus, Spumavirus, Bunyavirus, Hantavirus, Nairovirus, Phlebovirus, Tospovirus, Calicivirus, Enterovirus, Rhinovirus, Hepatovirus, Cardiovirus, Aphthovirus, Astrovirus, Flavivirus, Pestivirus, Hepacivirus, Alphanodavirus, Coronavirus, Torovirus, Alphavirus, Arterivirus, and Deltavirus; and/or (c) a virus species from the virus family in (a) or the virus genus in (b); wherein the set of oligonucleotides as a whole can detect any vertebrate virus. In one aspect, the amino acid sequences are selected from the CD-ROM Table Appendix.

With respect to any aspect of the invention that relates to amino acid sequences that comprise a motif conserved in a virus taxon, the motif can comprise, for example, a portion of a polymerase or a structural region. The structural region can be a capsid protein. The motif can comprise a sequence of a non-coding region. The non-coding region can comprise a portion of a cis-regulatory region. The cis-regulatory region can comprise binding sites from transcription factors, proteases, ribosomes, or other proteins. The cis-regulatory region can comprise a portion of an internal ribosomal entry site (IRES).

In one aspect, the invention provides a set of oligonucleotides that can hybridize to any vertebrate virus, wherein the set of oligonucleotides comprise sequences reverse-translated from conserved motifs, wherein the conserved motifs do not comprise more than 3 motifs conserved in any vertebrate virus species.

In one aspect, the invention provides a set of oligonucleotides that can hybridize to any vertebrate virus, wherein the set of oligonucleotides comprise sequences reverse-translated from conserved motifs, and wherein the set of oligonucleotides do not comprise more than 10,000 different sequences.

With respect to any aspect of the invention that refers to the amino acid sequences from the CD-ROM Table Appendix, such amino acid sequences can comprise: (1) conservative mutations; and/or (2) sequences that are at least 10 amino acid residues in length and are at least 90, 95, 96, 97, 98, or 99% identical to one of the sequences listed in the CD-ROM Table Appendix.

In one aspect, the invention provides a set of oligonucleotides for the detection of a vertebrate virus, the set of oligonucleotides comprising a plurality of nucleotide sequences, wherein each nucleotide sequence is at least 20 nucleotides in length and has at least 90% (or 95, 96, 97, 98, 99%) sequence identity to a portion of a sequence listed in the CD-ROM Table Appendix. The set of oligonucleotides can be for the detection of a virus from a particular vertebrate virus family, genus, or species, and the sequences can therefore be selected from sequences in the CD-ROM Table Appendix that corresponds to the desired virus family, genus, or species.

In one aspect, the invention provides a set of oligonucleotides for the detection of vertebrate viruses, the set of oligonucleotides comprising from about 500 (or from about 1000, 1500, 2000, 2500, 3000, 3500, 4000, 5000, 7500, 8000, 8500) to about 10,000 different oligonucleotide sequences, wherein each oligonucleotide of the set comprises a sequence that is reverse translated from an amino acid sequence (or portion thereof) from the CD-ROM Table Appendix.

In one aspect, the invention provides a method for designing an oligonucleotide for viral screening, the method comprising: (a) compiling a database of viral sequences, wherein the database of viral sequences comprises nucleotide sequences and amino acid sequences; (b) classifying each nucleotide sequence and amino acid sequence into a viral order, family, genus, and species; (c) identifying from the database of viral sequences a set of amino acid sequences wherein each amino acid sequence of the set comprises a protein domain or motif, (d) identifying from the set of amino acid sequences of step (c) a subset of amino acid sequence motifs that are conserved throughout a viral family, genus, and/or species; (e) determining the nucleotide sequences coding for the subset of amino acid sequence motifs of step (d), wherein the nucleotide sequences coding for the subset are obtained from the database of viral sequences; and (f) designing a group of oligonucleotides comprising nucleotide sequences selected from the nucleotide sequences coding for the subset of amino acid sequence motifs.

In one aspect, the designing of step (f) comprises using a set covering algorithm to determine the minimum number of sequences that needs to be selected from the nucleotide sequences coding for the subset of amino acid sequence motifs in order to represent every viral species in the viral database. In one aspect, in step (f), the group of oligonucleotides can comprise nucleotide sequences selected from nucleotide sequences that code for amino acid sequence motifs conserved in a single viral family or in a single viral genus.

In one aspect, the viral sequence database consists essentially of sequences classified to be from vertebrate viruses. In one aspect, the viral sequence database comprises sequences for partial genomes of a viral species or for partial coding sequences for a viral protein. In one aspect, the viral database comprises sequences from at least 25, 50, 75, or 100 species of vertebrate viruses. In another aspect, the nucleotide sequences for a viral species comprises sequences from more than one representative genome of the virus species. In another aspect, the viral sequence database does not comprise sequences from viruses that infect plants or bacteria.

In one aspect, the compiling step comprises obtaining a nucleotide sequence or amino acid sequence identified to be viral from one or more public sequence collections, wherein the public sequence databases comprise GenBank®; DNA DataBank of Japan (DDBJ); the European Molecular Biology Laboratory (EMBL); Reference Sequence (RefSeq) collection; translated coding regions from DNA sequences in GenBank, EMBL, and DDBJ; Protein Information Resource (PIR); SWISS-PROT; Protein Research Foundation (PRF); and Protein Data Bank (PDB); and any successor entity.

In one aspect, the classifying step further comprises classifying each nucleotide sequence and amino acid sequence into a viral subfamily, serogroup, subspecies, and/or isolate. The classifying step can be based on, for example, viral taxonomic tree criteria from the International Committee on the Taxonomy of Viruses.

In one aspect, wherein in step (c) (i.e, identifying from the database of viral sequences a set of amino acid sequences wherein each amino acid sequence of the set comprises a protein domain or motif), at least 150,000 different amino acid sequences are identified to comprise a protein domain or a portion of a protein domain. In another aspect, the identifying in step (c) comprises using Hidden Markov Models (HMMs). In another aspect, the protein domain or motif in step (c) comprises a Pfam domain or portion thereof.

In one aspect, wherein step (d) (i.e., identifying from the set of amino acid sequences of step (c) a subset of amino acid sequence motifs that are conserved throughout a viral family, genus, and/or species) comprises using a probabilistic model for identifying from the set of amino acid sequences of step (c) the subset of amino acid sequence motifs that are conserved throughout a viral family, genus, and/or species. The probabilistic model can be, for example, a MEME algorithm.

In one aspect, the invention provides a microarray comprising any one of the sets of nucleic acids of the invention. In one aspect, the invention provides a microarray comprising the nucleotide sequences listed in the CD-ROM Table Appendix (or complementary sequences thereof, or sequences that are at least 90% identical to the sequences listed in the Table Appendix). In one aspect, the invention provides a microarray comprising nucleotide sequences reverse-translated from the amino acid sequences listed in the CD-ROM Table Appendix.

In one aspect, the invention provides a method for identifying a virus from a environmental or clinical sample, the method comprising: (a) isolating nucleic acids from a sample containing the virus; (b) labeling the nucleic acids with a label; (c) hybridizing the labeled nucleic acids to a set of nucleic acids of any one of the sets described herein; and (d) identifying the nucleic acids from the set of nucleic acids that hybridized to the labeled nucleic acids, thereby identifying the virus. The isolated nucleic acids from the sample can be amplified by PCR. The virus from the sample can also be grown/expanded in culture prior to isolating nucleic acids. Alternatively, the isolated nucleic acids from the sample can be directly assayed without amplification or culture expansion.

In one aspect, the invention provides a computer program product residing on a computer readable medium, the computer program product comprising instructions for causing a computer to: (a) compile a database of viral sequences, wherein the database of viral sequences comprises nucleotide sequences and amino acid sequences; (b) classify each nucleotide sequence and amino acid sequence into a viral order, family, genus, and species; (c) identify from the database of viral sequences a set of amino acid sequences wherein each amino acid sequence of the set comprises a protein domain or motif, (d) identify from the set of amino acid sequences of step (c) a subset of amino acid sequence motifs that are conserved throughout a viral family, genus, and/or species; (e) determine the nucleotide sequences coding for the subset of amino acid sequence motifs of step (d), wherein the nucleotide sequences are obtained from the database of viral sequences; and (f) design a group of oligonucleotides comprising nucleotide sequences selected from the nucleotide sequences coding for the subset of amino acid sequence motifs.

In one aspect, the invention provides a method for generating an antibody reactive to a plurality of different viruses, the method comprising: (a) compiling a database of viral sequences, wherein the database of viral sequences comprises nucleotide sequences and amino acid sequences; (b) classifying each nucleotide sequence and amino acid sequence into a viral order, family, genus, and species; (c) identifying from the database of viral sequences a set of amino acid sequences wherein each amino acid sequence of the set comprises a protein domain or motif, (d) identifying from the set of amino acid sequences of step (c) a subset of amino acid sequence motifs that are conserved throughout a viral family, genus, and/or species; (e) immunizing an animal with a peptide comprising a sequence motif from step (d) or a portion thereof, and (f) isolating serum from the animal comprising antibodies reactive to the peptide or an antibody producing cell from the animal that produces antibodies reactive to the peptide. The method can further comprise making a hybridoma cell from the antibody producing cell isolated in step (f).

In one aspect, the invention provides a system for at least one of diagnosis, surveillance, or discovery of infection or disease, the system comprising: a central processing unit (CPU); a storage device coupled to the CPU; a database of genetic information residing on the storage device, wherein the CPU checks a data for new genetic information and updates the database with the genetic information; and an input device coupled to the CPU which inputs a genetic sequence and hybridization results of the genetic sequence, wherein the CPU analyzes the hybridization results of the genetic sequence and generates information regarding the placement of the genetic sequence in the database.

In one aspect, the invention provides a method for updating a database of genetic information, comprising: downloading one or more sequences from at least one source of information at an interval; reconciling differences among the one or more sequences; determining if the one or more sequences should be added to the database; and storing the one or more sequences in the database. The determining can comprise determining if the sequence is covered, within a programmable difference of nucleotide mismatches, by at least one sequence already in the database.

In one aspect, the invention provides a method for maintaining the quality of a taxonomic database of genetic information, comprising: creating a subset of a collection of gene sequences; attempting hybridization with each member of the subset against the database; and deciding, based on the number of successful hybridizations of the subset against the database, to make changes to the database;

In one aspect, the invention provides a method for the evaluation of hybridization results, comprising: analyzing a pattern of positive signals in the hybridization results; eliminating signals from internal controls and position makers; and calculating a probability for the pattern to match a family, genus, and species.

In one aspect, the invention provides a method for generating one or more primers, comprising: generating a matrix from a set of one or more primers; building a tree from the matrix; applying a scoring function to the tree; and applying a set covering algorithm to the information from the scoring function. The generated primers can be, for example, degenerate primers. The building of the tree can comprise, for example: extracting sub-alignments from the entire alignment; filtering the sub-alignments for uniqueness; performing a pairwise comparison of the sub-alignments; and building the tree. The tree can be generated, for example, by using an algorithm that depends on Euclidean distance. The scoring function can comprise, for example: removing primers which are outside ranges of physical constraints; comparing the primers to sequences in the sub-alignment to determine the likelihood of hybridization; and assigning a score based on the said comparing. The set-covering algorithm can comprise, for example, a greedy algorithm.

In one aspect, the invention provides a method for generating one or more primers, comprising: selecting sequence information from a data source; performing multiple alignments; and designing primers. The data source can be a collection of DNA sequences, such as one of the databases described herein. The performing multiple alignment step can comprise, for example, using a general purpose multiple sequence alignment program. In one aspect, the primers can be designed for multiplex PCR.

### BRIEF DESCRIPTION OF THE FIGURES

FIG. 1. Flow-chart of one embodiment for a method to design a set of oligonucleotides to detect vertebrate viruses.

FIG. 2. Plot of confidence intervals over the number of successful predictions in a sample size of n=30. See Example 3.

FIG. 3. Analysis of the detected hybridization pattern and calculation of a ranked probability for that pattern to match a given viral family, genus and species based on the taxonomic information implemented in the integrated Greene database.

FIG. 4. Identification of an unclassified virus isolate by GreeneChip1.1. RNA from an unclassified virus isolate was analyzed by GreeneChip 1.1 and identified by a prototype analysis tool as ‘Sindbis virus’ (FIG. 3 and Table 9). Hybridized sequences were eluted from the chip and sequenced. Sequence analysis identified the ‘unclassified’ isolated stored in the repository as a Sindbis isolate that had recently been characterized (GenBank Acc. AF429-428).

FIG. 5. Schematic of a primer design method (see Example 6). Sequences in a window the size of the desired primer (1) are compared to generate a similarity matrix (2), which is then used to build a phylogenetic tree (3). The consensus sequence for each branch of the tree is determined, then scored (4). Primers which do not pass the criteria are filtered out. A matrix corresponding to the ability of a primer to amplify a template is constructed, where 1 is true and 0 is false (5). The matrix is used by the Set Covering Algorithm to determine the minimal set of primers which can amplify sequences in the window. Acceptable primers from the entire sequence are matched for Tm and grouped by amplicon size for the user to review.

FIG. 6. Multiplex PCR detection of Viral Hemorrhagic Fevers (see Example 6). Primers for Ebola Zaire, Crimean Congo Hemorrhagic Fever (CCHV), Seoul Hantavirus, Kyasanur Forest virus and Rift Valley Fever virus were mixed together and used to amplify Viral Hemorrhagic Fever (VHF) standards from a background of human DNA. Amplicons were separated by agarose gel electrophoresis and visualized by ethidium bromide staining. The multiplex PCR successfully amplified all standards at 500,000 and 50,000 copies; no false positives were detected.

FIG. 7. Running time of Greedy vs. Brute Force implementation of SCP (see Example 6). Computational time as a function of possible primers is plotted. Each datapoint represents the average of hundreds of trials; variance was minimal. Solutions for both algorithms were identical in all cases. Time data were fitted to exponential and logarithmic equations.

FIG. 8. Phylogeny of HAdV derived from Hexon fragment. A phylogenetic tree was constructed from an alignment of the PCR amplified region in the Hexon gene by neighbor joining followed by bootstrapping. Leaves of the tree were condensed in undifferentiated branches for clarity. HAdV probes were designed to identify individual serotypes in cases where they could be separated by molecular phylogeny (HAdV-12, -31, -18, -21, -7, -3, -8, -4, -5, -1, -6, -2, -40, an -41). In cases where bootstrap values between serotypes were low, probes were designed to identify the species, as in species D and B. HAdV-7 is separated into two genogroups; G1 is more related to HAdV-3 than G2.

FIG. 9. HAdV-5 Hybridization. A HAdV-5 reference strain and a clinical sample were amplified and hybridized to the array using the described protocol. (a) The array, Cy3 fluorescence (green) is generated by the labeled amplicon, Cy5 (red) is the quality control oligonucleotide. (b) Cy3 fluorescence only, HAdV-5 specific probes are boxed. (c) Average relative fluorescence of triplicate spots for hybridization in (b), error bars represent 1 standard deviation. (d) Hybridization data of clinical sample SO4367; posterior sequence analysis confirms it is a HAdV-5.

FIG. 10A-C. Sequences of HAdV Array. Probes were designed to identify specific HAdV serotypes (in bold, 14 total) when molecular phylogeny allowed differentiation. Three probes from different regions were designed to increase assay robustness by redundancy. Due to high similarity in Species B and D, specific probes could only be designed for HAdV-3, -7, -21, and -8.

FIG. 11. Identification of HAdV Reference Strains by Hybridization. Reference strains of Adenovirus from each species were hybridized to the array. Fluorescence data was normalized by percentage of maximum signal, and then spots were categorized by quartile. Asterisks (*) indicate highest hybridization signal.

FIG. 12. Identification of Adenovirus clinical Samples by Hybridization. 19 clinical samples of Adenoviruses were hybridized to the array. The clinical samples were serotyped by sequencing and molecular phylogeny to confirm the result. Fluorescence data was normalized by percentage of maximum signal, and then spots were categorized by quartile. Asterisks (*) indicate highest hybridization signal.

FIG. 13. Performance with clinical materials was tested using blood, sera or oral swaps from 24 human victims of VHF including 5 cases of Ebola hemorrhagic fever from the 1995 Kikwit outbreak, Democratic Republic of the Congo (DRC); 6 cases of Marburg hemorrhagic fever collected in 2000 during the Durba outbreak, DRC, and in 2005 in Uige, Angola; 4 cases of Lassa fever obtained in 2004 from Sierra Leone; 4 cases of Rift Valley fever from Namibia in 2004 and Kenya in 1998; and 5 cases of Crimean-Congo hemorrhagic fever from South Africa collected from 1986-93. Infection with the respective agent had been previously diagnosed through virus isolation, RT-PCR and in case of Lassa virus infections with antigen detection ELISA. Differential diagnosis by blinded MassTag PCR analysis was accurate in all cases.

### DETAILED DESCRIPTION OF THE INVENTION

As used herein, the following terms and phrases shall have the meanings set forth below. Unless defined otherwise, all technical and scientific terms used herein have the same meaning as commonly understood to one of ordinary skill in the art. The singular forms “a,” “an,” and “the” include plural reference unless the context clearly dictates otherwise.

The term “about” is used herein to mean approximately, in the region of, roughly, or around. When the term “about” is used in conjunction with a numerical range, it modifies that range by extending the boundaries above and below the numerical values set forth. In general, the term “about” is used herein to modify a numerical value above and below the stated value by a variance of 20%.

The term “conserved residue” refers to an amino acid that is a member of a group of amino acids having certain common properties. The term “conservative amino acid substitution” refers to the substitution (conceptually or otherwise) of an amino acid from one such group with a different amino acid from the same group. A functional way to define common properties between individual amino acids is to analyze the normalized frequencies of amino acid changes between corresponding proteins of homologous organisms (Schulz, G. E. and R. H. Schirmer, Principles of Protein Structure, Springer-Verlag). According to such analyses, groups of amino acids may be defined where amino acids within a group exchange preferentially with each other, and therefore resemble each other most in their impact on the overall protein structure (Schulz, G. E. and R. H. Schirmer, Principles of Protein Structure, Springer-Verlag). One example of a set of amino acid groups defined in this manner include: (i) a charged group, consisting of Glu and Asp, Lys, Arg and His, (ii) a positively-charged group, consisting of Lys, Arg and His, (iii) a negatively-charged group, consisting of Glu and Asp, (iv) an aromatic group, consisting of Phe, Tyr and Trp, (v) a nitrogen ring group, consisting of His and Trp, (vi) a large aliphatic nonpolar group, consisting of Val, Leu and Ile, (vii) a slightly-polar group, consisting of Met and Cys, (viii) a small-residue group, consisting of Ser, Thr, Asp, Asn, Gly, Ala, Glu, Gln and Pro, (ix) an aliphatic group consisting of Val, Leu, Ile, Met and Cys, and (x) a small hydroxyl group consisting of Ser and Thr.

In addition to the groups presented above, each amino acid residue may form its own group, and the group formed by an individual amino acid may be referred to simply by the one and/or three letter abbreviation for that amino acid commonly used in the art.

As used herein, the term “specifically hybridizes” refers to the ability of a nucleic acid probe/primer of the application to hybridize to at least 12, 15, 20, 25, 30, 35, 40, 45, 50 or 100 consecutive nucleotides of a target sequence, or a sequence complementary thereto, or naturally occurring mutants thereof, such that it has less than 15%, less than 10%, less than 5%, or less than 2% background hybridization to a cellular nucleic acid (e.g., mRNA or genomic DNA) other than the target gene. A variety of hybridization conditions can be used to detect specific hybridization.

Appropriate hybridization conditions are known to those skilled in the art or can be determined experimentally by the skilled artisan. See, for example, Current Protocols in Molecular Biology, John Wiley & Sons, N.Y. (1989), 6.3.1-12.3.6; Sambrook et al., 1989, Molecular Cloning, A Laboratory Manual, Cold Spring Harbor Press, N.Y; S. Agrawal (ed.) Methods in Molecular Biology, volume 20; Tijssen (1993) Laboratory Techniques in biochemistry and molecular biology-hybridization with nucleic acid probes, e.g., part I chapter 2 “Overview of principles of hybridization and the strategy of nucleic acid probe assays”, Elsevier, N.Y.; Tibanyenda, N. et al., Eur. J. Biochem. 139:19 (1984) and Ebel, S. et al., Biochem. 31:12083 (1992); Rees et al., Biochemistry 32: 137-144 (1993); Chakarbarti and Schutt, BioTechniques 32: 866-874 (2002); and SantaLucia and Hicks, Annu. Rev. Biomol. Struct. 33: 415-40 (2004).

As used herein, “identity” means the percentage of identical nucleotide or amino acid residues at corresponding positions in two or more sequences when the sequences are aligned to maximize sequence matching, i.e., taking into account gaps and insertions. Identity can be readily calculated by known methods, including but not limited to those described in (Computational Molecular Biology, Lesk, A. M., ed., Oxford University Press, New York, 1988; Biocomputing: Informatics and Genome Projects, Smith, D. W., ed., Academic Press, New York, 1993; Computer Analysis of Sequence Data, Part I, Griffin, A. M., and Griffin, H. G., eds., Humana Press, New Jersey, 1994; Sequence Analysis in Molecular Biology, von Heinje, G., Academic Press, 1987; and Sequence Analysis Primer, Gribskov, M. and Devereux, J., eds., M Stockton Press, New York, 1991; and Carillo, H., and Lipman, D., SIAM J. Applied Math., 48: 1073 (1988). Methods to determine identity are designed to give the largest match between the sequences tested. Computer program methods to determine identity between two sequences include, but are not limited to, the GCG program package (Devereux, J., et al., Nucleic Acids Research 12(1): 387 (1984)), BLASTP, BLASTN, and FASTA (Altschul, S. F. et al., J. Molec. Biol. 215: 403-410 (1990) and Altschul et al. Nuc. Acids Res. 25: 3389-3402 (1997)). The BLAST X program is publicly available from NCBI and other sources (BLAST Manual, Altschul, S., et al., NCBI NLM NIH Bethesda, Md. 20894; Altschul, S., et al., J. Mol. Biol. 215: 403-410 (1990). The Smith Waterman algorithm can also be used to determine identity.

The term “oligonucleotide” refers to a short nucleic acid molecule, e.g., a nucleic acid molecule having from about 10 to about 200 nucleotides. Oligonucleotides can be single stranded or double stranded.

The invention provides methods for the design of sets or groups of oligonucleotides that can be used to detect, identify, and/or analyze pathogens. The invention also provides compositions produced by these methods. The methods produce sets of oligonucleotides that can be comprehensive in their ability to detect pathogens from specified taxa. For example, the invention provides a vertebrate viral database that is comprehensive in terms of public and private viral sequences and accurate in terms of accepted taxonomy. The database can be culled to identify a subset of coding sequences, which can then be further culled to identify a subset of sequences comprising protein domains or Pfams. This protein domain comprising subset can then be analyzed to identify motifs in the subset that are conserved in one or more taxa. Given the need to detect pathogens from given taxa, oligonucleotides can be derived or reverse-translated from motifs conserved in the given taxa, such that the oligonucleotides as a group can provide comprehensive detection/analysis capability over pathogens in the given taxa. In one application, these sets of oligonucleotides can be used to identify biowarfare pathogens in public environments. In another application, these sets of oligonucleotides can be used to analyze pathogens from environmental or tissue samples.

Thus, the invention provides tools for the diagnosis of pathogenic infection, including without limitation the differential diagnosis of infection by influenza viruses, select NIAID priority agents, and agents that may cause signs and symptoms that mimic those due to infection with select agents. In one embodiment, the invention seeks to establish stable and sensitive viral detection/analysis kits for use in public health laboratories, hospital-based clinical laboratories, point-of-care settings, and in field-operations.

Sequence motifs can be identified or discovered by examining whether sequence patterns are conserved among different species such that the sequence patterns display evolutionary conservation. MEME (Multiple EM for Motif Estimation) and MAST (Motif Alignment and Search Tool) are two exemplary methods that can be used to identify sequence motifs within large amounts of sequence data.

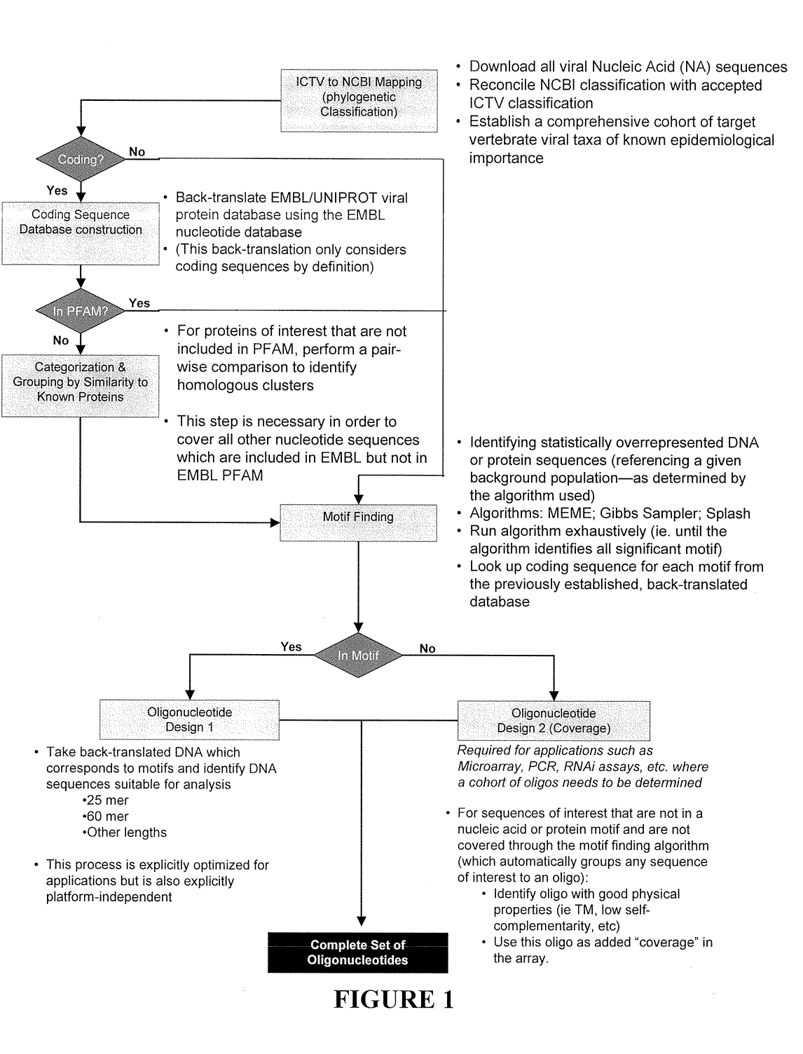

MEME represents motifs as position-dependent letter-probability matrices which describe the probability of each possible letter at each position in the pattern. Individual MEME motifs do not contain gaps. Patterns with variable-length gaps are split by MEME into two or more separate motifs. MEME takes as input a group of DNA or protein sequences (the training set) and outputs as many motifs as requested. MEME uses statistical modeling techniques to automatically choose the best width, number of occurrences, and description for each motif. In one embodiment, the invention provides sequence motifs obtained from the input of the amino acid sequences listed in Tables 1-5 provided in the CD-ROMs that accompany U.S. provisional patent application Ser. No. 60/861,365, filed Nov. 28, 2006 (the contents of which are hereby incorporated by reference in their entirety for all purposes). In another embodiment, the invention provides sequence motifs obtained from the input of sequences that comprise a PFAM as described herein and in FIG. 1.

MAST is a tool for searching biological sequence databases for sequences that contain one or more of a group of known motifs. MAST takes as input a file containing the descriptions of one or more motifs and searches a sequence database for sequences that match the motifs.

In one embodiment, the invention provides a method for increasing the simplicity and/or efficiency for identifying or discovering sequence motifs in a plurality of sequence data. For example, as described below and in FIG. 1 (at step 1), viral nucleic acid sequences can be downloaded from public databases. After reducing this group of sequences by selecting only coding sequences (FIG. 1, step 2), this group of coding sequences itself can be reduced by selecting only coding sequences (FIG. 1, step 4) that are in a Motif or Protein Domain Database, such as Pfam, ProDom, Prosite, BLOCKS, Prints, InterPro, TIGRFAMs, or a database that is generated using an Automatic Domain Decomposition Algorithm (ADDA). Coding sequences that contain a motif can then be analyzed (FIG. 1, step 6) to identify which motifs are conserved across viral taxa, for example, to identify motifs that are conserved in members of a particular viral family, genus, or species. In one embodiment, a minimum number of sequences can be obtained from a larger number of input sequences through the use of a heuristic method. In another embodiment, a dataset having a minimum number of sequences can be a dataset having any number of sequences that is reduced relative to the input sequence set.

Pfam is a collection of protein families and domains that contains sequence motifs of these families and domains by using multiple protein alignments and profile-Hidden Markov Models (HMMs). As used herein, a “Hidden Markov Model (HMM)” is a statistical model for any system that can be represented as a succession of transitions between discrete states. For example, with respect to protein sequences, the discrete states correspond to the successive columns of a protein multiple sequence alignment. In principle, HMMs can be developed from unaligned sequences by successive rounds of optimization, but in practice, protein profile HMMs are simply built from curated multiple sequence alignments. HMM searches resemble later round PSI-BLAST searches (although based on curated alignments), with position-specific scoring for each of the amino acid, insertion, and deletion over the length of the sequence. Scores are reported both in bits of information and as an E-value. PFAM models may be constrained to be non-overlapping with one another and thus are more likely to describe domains rather than full-length proteins.

ProDom is a comprehensive set of protein domain families automatically generated from the SWISS-PROT (a curated protein sequence database where sequences are annotated with descriptions of the function of a protein, its domains structure, post-translational modifications, variants, etc.) and TrEMBL (a computer-annotated supplement of Swiss-Prot that contains the translations of EMBL nucleotide sequence entries not yet integrated in Swiss-Prot. sequence databases). Prosite is a database of protein families and domains and contains patterns and profiles specific for more than a thousand protein families or domains. The blocks for the BLOCKS Database are made automatically by looking for the most highly conserved regions in groups of proteins documented in the Prosite Database. The Prosite pattern for a protein group is not used in any way to make the BLOCKS Database and the pattern may or may not be contained in one of the blocks representing a group. These blocks are then calibrated against the SWISS-PROT database to obtain a measure of the chance distribution of matches. It is these calibrated blocks that make up the BLOCKS Database. The PRINTS is a compendium of protein motif fingerprints. A fingerprint is a group of conserved motifs used to characterize a protein family. It is derived by the excision of conserved motifs from sequence alignments and refined by iterative dredging of the OWL, a non-redundant composite sequence database. InterPro is a database of protein families, domains and functional sites in which identifiable features found in known proteins can be applied to unknown protein sequences. TIGRFAMs (The Institute for Genomic Research Protein Families) are a collection of protein families featuring curated multiple sequence alignments, Hidden Markov Models (HMMs), and associated information designed to support the automated functional identification of proteins by sequence homology.

The methods of the invention can identify conserved regions in genomes of viruses, which regions can be used to design molecular and protein based assays for detection of known and unknown viruses. Applications include clinical microbiology; surveillance of blood products, tissues for transplantation, domestic animals and wildlife, and pathogen discovery. The methods of the invention use algorithms for sequence retrieval and analysis, pattern recognition and alignment. The methods of the invention can also involve manual modification based on human expertise in viral phylogenetics and molecular virology.

In certain embodiments, the invention provides methods for designing a set (or group or collection) of oligonucleotides that can be used to identify any unknown organism within a sample. In one embodiment, the organism is a microorganism. In one embodiment, the microorganism is a virus or a bacterium. In one embodiment, the virus is a vertebrate virus (i.e., a virus that infects vertebrates). In one embodiment, the virus is a plant virus. In one embodiment, the virus is a phage.

Sets of oligonucleotides that are designed by the methods of the invention can provide comprehensive coverage or, in other words, provide the ability to identify any unknown virus as long as the virus belong to a particular viral taxa. A particular viral taxon can be, for example, a family, subfamily, genus, subgenus, serogroup, subserogroup, species, subspecies, or isolate. The sets of oligonucleotides can be designed to provide comprehensive coverage for a particular viral taxon of interest. For example, in step 6 of FIG. 1, sequences containing PFAM motifs can be analyzed to identify motifs present in a particular viral taxon.

Compiling and Curating Databases

The methods for designing oligonucleotides involve a step of compiling a database of nucleotide and/or amino acid sequences (for example, see Step 1, FIG. 1). In one embodiment, a database is compiled with available viral nucleic acid sequences. For example, viral nucleic acid sequences (nucleotide and amino acid) can be obtained from: GenBank®; DNA DataBank of Japan (DDBJ); the European Molecular Biology Laboratory (EMBL); Reference Sequence (RefSeq) collection; translated coding regions from DNA sequences in GenBank, EMBL, and DDBJ; Protein Information Resource (PIR); SWISS-PROT; Protein Research Foundation (PRF); and Protein Data Bank (PDB); and any successor entity.

Once a database (whether viral, vertebrate viral, plant viral, phage viral, bacterial, etc.) is compiled, the sequences are then taxonomically and/or phylogenetically classified. Although public databases may already have provided a taxonomic or phylogenetic annotation to the sequences, public databases can often have serious classification errors. For example, NCBI (National Center for Biotechnology Information) viral sequences are classified by a NCBI Taxid, which corresponds to a phylogenetic classification. The NCBI Taxonomic database has serious errors in the classification of viruses, especially at the species level. The NCBI has a classification system for viruses that is not well curated, and differs from the ICTV (International Committee on the Taxonomy of Viruses) for many virus families. The NCBI viral taxonomy does not reflect accepted viral phylogeny because the classification of viruses is determined by the submitting author. This contributes to errors for three reasons: (1) the author may have erroneously classified virus, (2) no classification was available at the time of submission, or (3) historical changes in the nomenclature were not updated in the sequence record. Thus, in one embodiment, the invention provides a step of independently annotating a public database taxonomic annotation (such as the NCBI Taxid hierarchy) in order to reflect the accepted viral taxonomic tree, as accepted by the International Committee on the Taxonomy of Viruses (ICTV). The result of an independent step of annotation is a sequence database with correct phylogenetic relationships according to accepted standards, such as ICTV standards for a viral database. For a viral database, each genus can have different phylogenetic classes because the grouping of viruses varies because each genus can include clades, genotypes, serotypes, subgroups, or even geographical regions. Sequences from public databases that are taxonomically unclassified can be compared to known sequences in order to provide annotation.

Although the ICTV online database provides sequences that are representative of a subset of the taxonomic tree, it is not comprehensive. In contrast, in certain embodiments, the invention provides a comprehensive protein and nucleic acid sequence database that is built by extracting nucleic acid and protein sequences from the public repositories. For creating a viral database, each sequence is properly classified (i.e, ICTV designation) by the viral species it represents, along with any information about isolation and strain.

Creation of a sequence database of microorganisms can be important for developing diagnostic tests, screening assays, and epidemiology, among other purposes. The invention thus provides a sequence database that accurately reflects taxonomic relationships. In one embodiment, the invention provides a vertebrate viral database. In other non-limiting embodiments, the invention provides a plant virus database, a phage database, or a bacteria database. In one embodiment, the database of the invention reconciles two common errors in the NCBI classification: (1) sequences that are strains of a viral species but are classified as species themselves, and (2) sequences which are members of sub-genera groups but are not accurately classified. In one embodiment, such NCBI classification errors are corrected by manual curation for vertebrate viruses. Manual curation of a database requires specialists. For a vertebrate virus database, manual curation of the database requires specialists in viral phylogeny.

In one embodiment, databases of the invention include viral (or other pathogen) sequences from the Special Pathogens Branch (SPB) of the Centers for Disease Control. The sequences can be from source materials, such as isolates of select agents or clinical specimens. The CDC Special Pathogens Branch (SPB) virus collection includes a large number of VHF virus isolates that can be used for assay development (e.g., Ebola, Marburg, RVF, CCHF, Lassa, Machupo, Guanarito, Junin, Sabia, Nipah and HPS- and HFRS-associated hantaviruses).

TABLE 6
Samples available through the CDC Special Pathogens Branch
(SPB) virus collection

| Agent | Place | Year |
|---|---|---|
| Ebola or Marburg | | |
| Hemorrhagic Fever | | |
| Ebola Zaire | Dem. Rep. Congo | 1995 |
| Ebola Reston | Texas, Philippines | 1996 |
| Ebola Zaire | Gabon | 1996 |
| Marburg | Dem. Rep. Congo | 1999 |
| Ebola Sudan | Uganda | 2000-01 |
| Ebola Zaire | Gabon/Congo | 2001-03 |
| Ebola Sudan | Sudan | 2004 |
| Marburg | Angola | 2005 |
| Hantavirus Pulmonary | | |
| Syndrome (HPS) | | |
| HPS | southwestern US | 1993 |
| HPS | Paraguay, Argentina, Chile | 1995-97 |
| HPS | Panama | 2000 |
| Other | | |
| Machupo - Bolivian | Bolivia | 1994 |
| hemorrhagic fever | | |
| Lassa fever | Lagos, rural Nigeria | 1994 |
| Lassa fever | Sierra Leone | 1997 |
| Rift Valley fever | Kenya/Somalia/Tanzania | 1997-98 |
| Rift Valley fever | Saudi Arabia | 2000 |
| Crimean-Congo hemorrhagic fever | Kazakhstan &Oman | 1999 |
| Crimean-Congo hemorrhagic fever | Turkey | 2003-04 |
| Nipah | Malaysia/Singapore | 1999 |
| SARS | Asia > Global | 2003 |
| Transplant-associated | USA | 2004-05 |
| Lymphocytic choriomeningitis | | |

Updating Databases

Databases compiled as described above should be updated at regular intervals by downloading additional new sequences from the public databases. For example, in one embodiment, a viral database can be updated by downloading relevant information at least from GenBank/EMBL, SwissProt/UniProt, ISD (Influenza Sequence Database), ICTVdb, and PFAM. The downloaded information can be placed into a temporary integrated database and compared to the existing virus database (such as an integrated and curated ICTV/NCBI database). In one embodiment, the invention provides a programming script that queries GenBank/EMBL, SwissProt/UniProt, ISD and the CDC databases for new sequence entries at monthly intervals.

New sequence information can affect whether existing oligonucleotide sets need to be modified. For example, for a new sequence for a known viral species in a region represented on an oligonucleotide-coated array: The new sequence entry can be compared to probes on the existing array. No action is necessarily required if the sequence is covered by at least one existing probe with less than 4 nucleotide mismatches. New probes can be added if coverage is inadequate using the existing probe set. For a new sequence for a known viral species in a region not represented on the array: The new sequence entry can be used to query the integrated ICTV/NCBI virus database. If there is no related entry, or if homology to existing sequences is less than 90%, a new probe can be created. Without being bound by theory, the reasoning here is that divergence in one genome region not represented on the array may predict divergence in another region that is represented on the array.

Coding Sequence Database Construction

The database comprising publicly available nucleic acid sequences annotated (or curated or otherwise identified) to be viral (or other) in origin can be checked for coding sequence accuracy (for example, see Step 2, FIG. 1). In one embodiment, viral protein sequences from the Uniprot database can be cross referenced to the protein records in the EMBL nucleotide sequence database. Each protein record references a nucleotide record, from which the coding sequence (cDNA) can be extracted. The EMBL database has some errors in coding positions, so the DNA or RNA sequences can be validated by translating nucleotide sequences independently and comparing the independently translated sequences to the expected protein sequences. This will yield a “back-translated” viral protein database that can be used for motif determination.

Coding Sequence Delimitation

The compiled databases sequences that contain a coding sequence can be delimited or narrowed to a group of sequences that comprise a protein domain or portion thereof or a protein motif. One method for narrowing the coding sequence set to those sequences comprising a protein domain (or portion thereof) or a protein motif is to select only those coding sequences (or portions thereof) that are included in Pfam or other protein domain/motif database. Many viral proteins will have a described Pfam domain, some more than one. If a coding sequence that is not included in Pfam or other protein domain/motif database then a pair-wise comparison can be conducted to identify the sequence with respect to homologous clusters. The subset or group of viral sequences with Pfam domains (or portions thereof) or with sequences homologous to Pfam domains is then extracted or selected.

Motif Finding within the Group of Sequences Comprising Pfams

The complexity of an initial sequence database can be reduced by: (1) reducing the collection of database sequences to sequences comprising coding sequences or portions of coding sequences, and (2) reducing this coding sequence collection further to sequences comprising a protein domain or motif. This population of coding sequences that comprise a protein domain (or portion thereof) or a motif is then analyzed to identify statistically overrepresented DNA or protein sequence motifs within the population itself. Statistically overrepresented DNA or protein sequence motifs can be with respect to whether a DNA or protein sequence motif is statistically overrepresented in any level of an taxonomy (i.e., family, subfamily, genus, subgenus, serogroup, serotype, species, subspecies, isolate, etc.). The methods can use, for example, the information theoretical concept of Shannon's Entropy to identify regions of conservation and variability in a collection of sequences.

Motif funding can be used to identify sequence patterns within protein domains that are overrepresented in a level of taxonomy, thereby identifying motifs that are conserved across taxa or with in a taxon.

A probability calculating algorithm, for example, MEME, Gibbs Sampler, or Splash, can identify statistically overrepresented DNA or protein sequences. The algorithm can be run exhaustively, which means that the algorithm can be run until a desired number of significant motifs are identified. Statistically overrepresented protein motifs can be cross-referenced to the nucleotide coding sequences from the previously established back-translated database in order to design oligonucleotides.

In another embodiment, statistically overrepresented protein motifs can be cross-referenced to the nucleotide coding sequences in order to identify conserved peptides that can be used as immunogens for the generation of antibodies. It is possible that such antibodies can have a wide range of cross-reactivity across a taxon, where the cross-reactivity may potentially correspond to the level of taxonomic conservation displayed by the peptide sequence.

Oligonucleotide Design

The statistically overrepresented protein motifs can be cross-referenced to the nucleotide coding sequences from the previously established back-translated database in order to design oligonucleotides. In another embodiment, the protein motifs are not cross-referenced to the back-translated database to obtain nucleotide sequences for the motifs—rather oligonucleotides are designed in a degenerate fashion with respect to the motifs. In addition to the back-translated DNA or degenerate DNA that corresponds to the motifs, the oligonucleotides can further comprise nucleotide sequences that are upstream (5′) or downstream (3′) of the back-translated DNA.

In one embodiment, oligonucleotides can comprise from about 10 to about 250 oligonucleotides. In another embodiment, oligonucleotides can comprise from about 20 to about 65 nucleotides. In another embodiment, oligonucleotides can comprise about 25 or about 60 nucleotides.

In one embodiment, DNA that corresponds to the motifs are analyzed to determine which sequences are suitable for hybridization. Factors suitable for hybridization include, but are not limited to, identifying sequences: (1) having a high melting temperature, (2) little or no secondary structure, and (3) few homopolymeric stretches.

In another embodiment, DNA (or RNA) that corresponds to the motifs are analyzed to determine which sequences are suitable for a particular oligonucleotide-related application, such as microarray screening, PCR, and RNAi assays.

In one embodiment, oligonucleotides can be designed according to the algorithms and instructions described in Example 6. For example, in one embodiment, the invention provides a set of oligonucleotides that comprise the minimal number of oligonucleotides required to hybridize to any virus species in a specified taxon. The minimal number of oligonucleotides required to hybridize to any virus species in a specified taxon (i.e., a set of oligos providing comprehensive coverage of a taxon) can be determined by a Set Covering Algorithm, such as those described in Example 6.

Ensuring Comprehensive Coverage of a Taxon

A set of oligonucleotides can be designed as described above such that any organism of a particular taxon can be detected. In one embodiment, the invention provides a set of oligonucleotides that can detect any vertebrate virus. In another embodiment, the invention provides a set of oligonucleotides that can detect a virus that belongs to a particular vertebrate virus family. In another embodiment, the invention provides a set of oligonucleotides that can detect a virus that belongs to a particular vertebrate virus genus.

To ensure coverage of a taxon, oligonucleotides can be designed with respect to database sequences that are not represented by a motif. Sequences that are not represented by a motif can be analyzed to identify regions that may be conserved, where the analysis can be conducted by using probability matrices that can describe mutation rates, such as PAM250 and BLOSUM. A mutation matrix can be used to identify protein stretches that have a low probability of mutation, these stretches can then be back translated and used to design oligonucleotides to complement the motif-based oligonucleotides thereby ensuring coverage.

In one embodiment, the invention provides methods for identifying regions of conservation and variability. In another embodiment, the invention provides the below-described methods for identifying regions of conservation and variability by conducting sequence alignments of portions of genomes from databases that have not been reduced to smaller sets comprising coding sequences and PFAMs.

Thus, in one embodiment, the methods use the information theoretical concept of Shannon's Entropy to identify regions of conservation and variability in viral genomes. A curated, aligned database of viral genomes from public and private sequences is maintained to reflect diversity and phylogeny. Oligonucleotides for typing and subtyping of viruses are selected by software for specificity and minimal cross-reactivity. This method has general applicability for a wide variety of platforms including but not restricted to PCR, microarrays, and multiplex bead based assays (e.g. Luminex technologies). Its utility has been proven in assays wherein oligonucleotide targets were spotted onto glass slides to create DNA microarrays. Arrays were hybridized to a probe from a PCR or non specific amplification reaction, yielding signal when the probe is homologous to the target. This approach can in be extended to the identification of any nucleic acid.

Shannon's Entropy is the measure of variability in a system. In the case of DNA, there are 4 discrete states, corresponding to the nucleotides. The Shannon Entropy is the shortest binary encoding of the states of a variable. The formula is H(x)−E px log 2 where px is the probability of a given state (Shannon 1948, Cover and Thomas 1991). Alignments of viral genomes (or alignments of viral PFAMs) are made using sequences deposited in public databases. Where these databases are inadequate (e.g., there is only one representative of a given serotype or genomic sequence is incomplete) additional sequences are obtained primarily from either infected animals or cultured cells. Recent examples where this has been required include flaviviruses, bunyaviruses, and enteroviruses. Subregions of the alignments are chosen for conservation or variability based on the Entropy metric by evaluating each position of the alignment. Also, subregions are chosen by the knowledge of viral biology that will allow speciation. To create alignments which represent known sequence in a particular area, a representative seed alignment is used to query the database by BLAST for homologous viral sequences. In cases where the automated retrieval is unsatisfactory, sequences homologous sequences can be manually retrieved from the databases. The sequences can be classified according to virus phylogeny along the lines of the ICTV scheme.

The subregions are analyzed in parallel by software which implements the Entropy metric to quantify variability. A key objective of the method is to identify targets for specific purposes, such as forward and reverse primers for PCR, reporter oligonucleotides for real rime PCR, oligonucleotides for various microarray formats where different lengths are employed. Thus, a sliding window of 25, 50, 60, 70 nucleotides is used to evaluate every possible target.

In one embodiment, two categories of oligonucleotides are chosen based on their potential to (i) capture broad viral taxa including unknown viruses (e.g., genus specific targets) or (ii) allow discrete speciation of viral taxa (e.g., serotype or strain-specific targets). Whereas the former approach facilitates broad range surveillance and pathogen discovery, the latter facilitates molecular epidemiology and microbial forensics. In selecting broad targets, regions which are highly conserved (low entropy scores, connoting a similar makeup of nucleotides among the strains) are chosen. A degenerate oligonucleotide is determined, similar to consensus PCR, though fully automated programs. The degenerate target design algorithm maximized the chance for hybridization with members of a genius while minimizing the number of degenerate positions.

A refining algorithm chooses minimized degeneracy based on the propensity for nucleotide changes to occur together between strains. Serotype and strain-specific oligonucleotide are determined by an algorithm which identifies speciating areas. By evaluating the contribution of a family to the overall Entropy (variability) of a virus taxon, regions are selected which are conserved in the family but variable in the rest of the genus. The algorithm maximizes intrafamily similarity to the target while minimizing extra-family similarity. Filtering of the potential targets examines critical performance characteristics including Tm, hairpin formation and self-annealing.

Compositions for Detecting Vertebrate Viruses

Some of the potential application of this invention include, for example: detection and differentiation of microorganism and host transcripts in clinical, environmental, and food samples; genetic compatibility studies; screening of blood and transplantation products; and forensics. One of the problems this invention solves is dealing with the sensitive, multiplex detection and characterization of genetic targets where precise target sequence might not be known. The invention provides an advantage, for example, in that it does not only consider completely sequenced viral genomes. It considers both completely and incompletely sequenced species. The invention also includes more than one species representative because, for example, sequences can be considerably divergent within a species.

In one embodiment, the invention is useful as a tool for identifying oligonucleotide sequences that hybridize to related but not necessarily identical sequence targets. In one embodiment, the methods of the invention can be used to design primers for consensus PCR and targets for DNA microarrays. Other non-limiting potential applications of the invention include, for example: detection and differentiation of microorganism and host transcripts in clinical, environmental, and food samples; genetic compatibility studies; screening of blood and transplantation products; forensics; and bio-safety testing of pharmaceutical compounds. Because oligonucleotides designed by the invention are based on sequence conservation, the invention provides methods using such oligonucleotides for sensitive, multiplex detection and characterization of genetic targets where precise target sequence might not be known.

In one embodiment, the methods of the invention for designing oligonucleotides allow for the creation of sets of oligonucleotides that can detect or identify viruses in a sample.

In one embodiment, the invention provides a set of oligonucleotides that can be used to detect any vertebrate virus in a sample.

In one embodiment, the invention provides a set of oligonucleotides that can provide comprehensive coverage of vertebrate viruses, wherein the set of oligonucleotides comprise the nucleic acid sequences listed in the CD-ROM Table Appendix.

In another embodiment, the invention provides a set of oligonucleotides that can provide comprehensive coverage of vertebrate viruses, wherein the set of oligonucleotides comprise nucleic acid sequences derived or reverse-translated from the amino acid sequences listed in the CD-ROM Table Appendix.

In another embodiment, the invention provides sequence motifs that are derived or obtained from the amino acid sequences listed in the CD-ROM Table Appendix. These sequence motifs are then compared to nucleic acid sequences in NCBI or ICTV databases or the NCBI/ICTV integrated database in order to identify viral nucleic acid sequences that may code for the sequence motifs. Oligonucleotides can be designed based on the sequence motifs, where the design can include degenerate sequences, conservative mutations, and sequence variation such that the oligonucleotides are at least 90, 95, 96, 97, 98, or 99% identical to the nucleotide sequences in NCBI or ICTV databases that code for the motifs.

In one embodiment, the invention provides a set of oligonucleotides for detecting vertebrate viruses, where the set of oligonucleotides are reverse translated from amino acid sequences, where each amino acid sequence comprises a motif conserved in either: (a) a virus family, wherein the virus family is selected from the group consisting of: Adenoviridae, Arenaviridae, Arteriviridae, Asfarviridae, Astroviridae, Birnaviridae, Bornaviridae, Bunyaviridae, Caliciviridae, Circoviridae, Coronaviridae, Deltavirus, Filoviridae, Flaviviridae, Hepadnaviridae, Hepatitis E-like viruses, Herpesviridae, Infectious laryngotrachetitis-like viruses, Iridoviridae, Nodaviridae, Orthomyxoviridae, Papillomaviridae, Paramyxoviridae, Parvoviridae, Picornaviridae, Polyomaviridae, Poxyiridae, Reoviridae, Retroviridae, Rhabdoviridae, and Togaviridae; (b) a virus genus, wherein the virus genus is selected from the group consisting of: Asfivirus, Orthopoxvirus, Parapoxvirus, Avipoxvirus, Capripoxvirus, Leporipoxvirus, Suipoxvirus, Molluscipoxvirus, Yatapoxvirus, Entomopoxvirus A, Entomopoxvirus B, Entomopoxvirus C, Iridovirus, Chloriridovirus, Ranavirus, Lymphocystivirus, Simplexvirus, Varicellovirus, Cytomegalovirus, Muromegalovirus, Roseolovirus, Lymphocryptovirus, Rhadinovirus, Ichnovirus, Bracovirus, Polyomavirus, Papillomavirus, Mastadenovirus, Aviadenovirus, Orthoreovirus, Orbivirus, Rotavirus, Coltivirus, Aquareovirus, Cypovirus, Fijivirus, Phytoreovirus, Oryzavirus, Aquabimavirus, Avibimavirus, Entomobirnavirus, Influenzavirus A, Influenzavirus B, Influenzavirus C, Influenzavirus D, Paramyxovirus, Morbillivirus, Rubulavirus, Pneumovirus, Bornavirus, Marburgvirus, Ebolavirus, Arenavirus, Alpharetrovirus, Betaretrovirus, Gammaretrovirus, Type D Retrovirus group, Deltaretrovirus, Epsilonretrovirus, Lentivirus, Spumavirus, Bunyavirus, Hantavirus, Nairovirus, Phlebovirus, Tospovirus, Calicivirus, Enterovirus, Rhinovirus, Hepatovirus, Cardiovirus, Aphthovirus, Astrovirus, Flavivirus, Pestivirus, Hepacivirus, Alphanodavirus, Coronavirus, Torovirus, Alphavirus, Arterivirus, and Deltavirus; and/or (c) a virus species from the virus family in (a) or the virus genus in (b); wherein the set of oligonucleotides as a whole can detect any vertebrate virus. In one embodiment, the amino acid sequences are selected from the CD-ROM Table Appendix.

In one embodiment, the invention provides a set of oligonucleotides for detecting vertebrate viruses from a particular family, where the set of oligonucleotides are reverse translated from amino acid sequences, where each amino acid sequence comprises a motif conserved in the virus family to be detected, wherein the virus family is selected from the group consisting of: Adenoviridae, Arenaviridae, Arteriviridae, Asfarviridae, Astroviridae, Birnaviridae, Bornaviridae, Bunyaviridae, Caliciviridae, Circoviridae, Coronaviridae, Deltavirus, Filoviridae, Flaviviridae, Hepadnaviridae, Hepatitis E-like viruses, Herpesviridae, Infectious laryngotrachetitis-like viruses, Iridoviridae, Nodaviridae, Orthomyxoviridae, Papillomaviridae, Paramyxoviridae, Parvoviridae, Picornaviridae, Polyomaviridae, Poxyiridae, Reoviridae, Retroviridae, Rhabdoviridae, and Togaviridae. The motif conserved in the virus family can include motifs that are conserved in genera or in species of the family. In one embodiment, the amino acid sequences are selected from the appropriate virus family table from the CD-ROM Table Appendix.

In one embodiment, the invention provides a set of oligonucleotides for detecting vertebrate viruses from a particular genus, where the set of oligonucleotides are reverse translated from amino acid sequences, where each amino acid sequence comprises a motif conserved in the virus genus to be detected, wherein the virus genus is selected from the group consisting of: Asfivirus, Orthopoxvirus, Parapoxvirus, Avipoxvirus, Capripoxvirus, Leporipoxvirus, Suipoxvirus, Molluscipoxvirus, Yatapoxvirus, Entomopoxvirus A, Entomopoxvirus B, Entomopoxvirus C, Iridovirus, Chloriridovirus, Ranavirus, Lymphocystivirus, Simplexvirus, Varicellovirus, Cytomegalovirus, Muromegalovirus, Roseolovirus, Lymphocryptovirus, Rhadinovirus, Ichnovirus, Bracovirus, Polyomavirus, Papillomavirus, Mastadenovirus, Aviadenovirus, Orthoreovirus, Orbivirus, Rotavirus, Coltivirus, Aquareovirus, Cypovirus, Fijivirus, Phytoreovirus, Oryzavirus, Aquabimavirus, Avibimavirus, Entomobirnavirus, Influenzavirus A, Influenzavirus B, Influenzavirus C, Influenzavirus D, Paramyxovirus, Morbillivirus, Rubulavirus, Pneumovirus, Bornavirus, Marburgvirus, Ebolavirus, Arenavirus, Alpharetrovirus, Betaretrovirus, Gammaretrovirus, Type D Retrovirus group, Deltaretrovirus, Epsilonretrovirus, Lentivirus, Spumavirus, Bunyavirus, Hantavirus, Nairovirus, Phlebovirus, Tospovirus, Calicivirus, Enterovirus, Rhinovirus, Hepatovirus, Cardiovirus, Aphthovirus, Astrovirus, Flavivirus, Pestivirus, Hepacivirus, Alphanodavirus, Coronavirus, Torovirus, Alphavirus, Arterivirus, and Deltavirus. The motif conserved in the virus genus can include motifs that are conserved in genus or in species of the genus. In one embodiment, the amino acid sequences are selected from the appropriate virus genus from the appropriate family table from the CD-ROM Table Appendix.

Detection Assays

Rapid, accurate differential diagnosis is critical to control of contagion and clinical management of infectious diseases. Syndromes are rarely specific for single pathogens, particularly early in the course of infection. Thus, diagnostic tools must simultaneously consider multiple agents. The methods of the invention can design sets of oligonucleotides that can be used to detect any agent within various taxa. For example, if a device is desired for the detection of any virus in the families Picornaviradae and Adenoviridae, then the invention can provide a set of oligonucleotides that can provide coverage of both families. Such a set of oligonucleotides can be based on conserved sequence motifs such that the collection of motifs ensure coverage of every species in both families.

Molecular methods for direct detection of microbial pathogens in clinical specimens are rapid, sensitive and may succeed where fastidious requirements for agent replication or the need for high level biocontainment confound cultivation. Various methods are employed or proposed for cultivation-independent characterization of infectious agents. These can be broadly segregated into methods based on direct analysis of microbial nucleic acid sequences (e.g., cDNA microarrays, consensus PCR, representational difference analysis, differential display), direct analysis of microbial protein sequences (e.g., mass spectrophotometry), immunological systems for microbe detection (e.g., expression libraries, phage display), and host response profiling. These prior methods can be enhanced with the use of the methods and oligonucleotide collections of the invention.

Consensus PCR (cPCR) has been a remarkably productive tool for biology. In addition to identifying pathogens, this method has facilitated identification of a wide variety of host molecules, including cytokines, ion channels, and receptors. One difficulty in applying cPCR to pathogen discovery in virology has been that it is difficult to identify conserved viral sequences of sufficient length to allow cross-hybridization, amplification, and discrimination in a traditional cPCR format. Domain-specific differential display attempts to overcome this problem, where the method employs short, degenerate primer sets designed to hybridize to viral genes that represent larger taxonomic categories than can be resolved in cPCR. Although this modification allowed the identification of the West Nile virus as the causative agent of the 1999 New York City encephalitis outbreak, it did not resolve issues of low throughput with cPCR due to limitations in multiplexing.

To address the need for highly multiplexed real time assays, Mass Tag PCR was created, which is a new PCR platform wherein digital mass tags rather than fluorescent dyes serve as reporters. The first description of this method was published in the context of a panel that distinguishes between 22 different viral and bacterial respiratory pathogens. However, Mass Tag PCR is not sufficient in instances where larger numbers of known pathogens must be considered, new but related pathogens are anticipated, or there is risk that sequence divergence can impair binding of PCR primers. To remedy these deficiencies, the invention provides for the design of sets of oligonucleotides that provide a higher tolerance for sequence divergence in part due to their foundation in sequence conservation.

Viral microarrays have potential to provide a platform for highly multiplexed differential diagnosis of infectious diseases. The number of potential features far exceeds that with any other known technology. Furthermore, sequence targets of up to 70 nucleotides are not uncommon. Thus, there is capacity to capture a wide variety of viral targets. Lastly, one can incorporate both microbial and host gene targets. This affords an opportunity to both detect viral agents and assess host responses for signatures consistent with various classes of infectious agents. In various embodiments, the invention provides viral microarrays that comprise any one of the sets of oligonucleotides described herein, or any of one of the sets of oligonucleotides that can be designed by the methods described herein.

### EXAMPLES

### Example 1

### A Vertebrate Viral Database and Microarrays

Because vertebrate viruses are highest priority for human disease, a vertebrate viral database was first constructed, with a plan to later extend the database to viruses of invertebrates, plants, and prokaryotes. A database was compiled that included (1) every vertebrate virus listed in the ICTV database (ICTVdb), and (2) non-published sequences from private collections (i.e., Centers for Disease Control, etc.). The NCBI database is not exhaustively curated; thus, it contains many entries where annotation is missing, outdated, or inaccurate. An additional difficulty is that only incomplete sequence is available for many viruses where genomic sequencing efforts have received less emphasis or data are confined to networks focused on biodefense and emerging infectious diseases (e.g., WHO network and Department of Defense laboratories).

To circumvent limitations in curation and nomenclature in the NCBI database, and to eliminate the need for supercomputing in establishment of multiple alignments at the nucleotide (nt) level, the construction of vertebrate viral database was conducted by using the PFAMs and HMMs. The NCBI database contained at the time of analysis a total of 291,571 viral sequences. Eighty-four percent of viral protein coding sequences in NCBI were represented in the PFAM database; the remaining 16% were mapped to this set using BLAST. The PFAM database is an assembly of sequence alignments, currently listing 7973 specific protein domains or ‘families’ defined through their sequence homology.

Sequences for the design of oligonucleotides were selected from these families based on biological parameters, including the degree of conservation of proteins or domains, their expression level during infection, and the amount of data available for the respective region. Where possible, one highly conserved region within the coding sequence of an enzyme such as a polymerase, and two more variable regions within structural proteins. Although more conserved sequences offer the most economic approach to global detection, RNAs encoding structural proteins can be present at higher levels than those encoding proteins needed only in catalytic amounts. In addition, a redundancy of three probes targeting noncontiguous sites along the genome might allow detection of naturally occurring or engineered chimeric viruses. This selection process yielded a database comprising 151,039 PFAM sequences that cover every family or genus in the ICTV database, listing vertebrate viruses for which complete or partial sequence is available with a redundancy of three genomic target regions whenever possible.

To condense the PFAM selection to a tractable number suitable for printing on common arrays that allow for 10,000 to 20,000 features, two approaches were taken. First, in instances where one viral species is associated with a large number of PFAM hits, such as HIV which is associated with approximately 80,000 PFAM hits, the selection of oligonucleotides was limited to highly conserved regions, without distinguishing subtypes, lineages, or serotypes. Second, long target regions were selected, for example, oligos designed to 60mer target regions were selected and were allow up to 4 nucleotide mismatches between entries in the viral database and the 60mer targets. This decision was based on experimental data (see first array discussion below) wherein the following were established: (1) array performance was better with 60mer than with 50mer targets, (2) 70mer targets were more difficult and expensive to synthesize and did not improve performance, and (3) hybridizations of intentionally mismatched target/sample pairs indicated tolerance of up to 8 mismatched bases distributed throughout the 60mer. Tolerance was still greater when mismatches were confined to the termini of the target.

Although substantial viral coverage can be achieved by using several Mass Tag panels, there are nonetheless instances where larger numbers of known pathogens must be considered, new but related pathogens are anticipated, or there is risk that sequence divergence can impair binding of PCR primers. To address this challenge, the vertebrate viral database was used to construct a viral array uniquely suited to diagnostic use with clinical materials.

The First Test Array

Initial viral array studies were conducted by spotting 50, 60, and 70 nucleotide long oligonucleotides representing a wide range of bunyaviruses and adenoviruses, with and without amino modifications at the 5′ end, on poly L-lysine and epoxy-coated glass slides. No difference between poly L-lysine or epoxy coatings, or between unmodified 60 or 70 nucleotide oligonucleotide targets were observed. However, hybridization signal improved with the increase in target length from 50 to 60 nucleotide for unmodified oligonucleotides, or amino modification. The enhanced signal with amino acid modification can be due to controlled binding of the target to the slide at one end of the molecule, such that the remainder of the target is free for hybridization. Unfortunately, the cost for amino acid modification can become prohibitive as the size of the library of targets increases. Thus, a first array was produced using 60 nucleotide oligonucleotides representing 1-2 sequences for each of the 1710 vertebrate viruses in the ICTVdb. This array of 3418 targets was used to establish conditions for amplification, labeling, and detection of viral sequences in clinical materials. A subset of targets in this slide was designed to include mismatches to bona fide viral sequences at the 5′ or 3′ end, or interspersed throughout the target. Analysis of these chimeric targets revealed that under standard conditions, hybridization signal could be achieved with up to 8 nucleotide mismatches randomly distributed throughout the target molecule. Tolerance was higher still when mismatching was confined to the termini. Based on these experiments, oligonucleotides can be conservatively designed such that each array contains targets that address known viral sequences with no more than 4 nucleotide mismatches.

The Second Test Array (GreeneChip 1.1)

In silica experiments were pursued and a total of 9066 oligonucleotides (60mers) were identified that cover vertebrate viruses in the integrated ICTV/NCBI virus database (1710 species, including reported isolates) in 3 gene regions, with 4 or fewer nucleotide mismatches. A panel of 9366 oligonucleotides, including 300 additional probes targeting host gene markers, was used to create the second test array.

To acquire additional information as described below through microarrays, 300 additional targets were selected, where the targets represented: the genes associated with cytokines, chemokines, and their receptors; components of the interferon-inducible signaling pathways; immunoglobulins (Ig) and Ig receptors; toll-like receptors and their downstream signaling pathways; complement components; MHC molecules; and heat shock proteins from a set of validated oligonucleotides. In applications of microarrays to an infectious disease outbreak of unknown cause, identification of signal(s) representing a single pathogen in samples from affected subjects is a primary objective. Implication of an agent in disease, however, requires far more than the demonstration of a molecular signature, and can be bolstered by coterminous evidence of gene expression consistent with host immune response to infection. Some acute samples evaluated using the microarray described herein platform can also show signs of coinfection; by examining profiles of signals representing infectious agents and host immune response genes across samples, agents involved in pathogenesis can be more easily distinguished from those that are preexisting or commensal. Furthermore, inclusion of oligonucleotides representing genes associated with innate and adaptive immune pathways can facilitate the application of microarray platforms in chronic diseases. To establish whether a chronic condition is induced or exacerbated by infection, the presence of dysregulated immune responses, in conjunction with low-level signal that is suggestive of a specific pathogen, can assist in defining the role of infectious agents in pathogenesis. Finally, in cases where clear evidence of any known pathogen is hard to find, or where signal is low, the presence of a profile consistent with immune activation can be helpful in determining whether to pursue additional studies focused on pathogen discovery.

After initial success with a 3418-target version of the array (the first test panel), additional bioinformatics analyses were performed to create a more comprehensive array. Oligonucleotides for this second generation array were selected to represent three distinct genomic target regions for every family or genus of vertebrate virus in the ICTVdb. Through an iterative process 98,310 sequences (151,039 PFAM domains) were identified that included available sequence data (complete or partial) for vertebrate viruses registered by the ICTV. Due to practical considerations, in silica experiments were conducted to further refine this library to a tractable number—9066 oligonucleotides (60mers) were selected that cover vertebrate viruses in the database (1710 species, including reported isolates) in 3 gene regions, with 4 or fewer nucleotide mismatches. Three hundred host gene targets were added to enhance functionality and provide clues to pathogenesis. This 9366 oligonucleotide panel was used to create “GreeneChip1.1” (see list of viral and host targets contained in CD-ROM Table Appendix filed along with U.S. provisional patent application Ser. No. 60/861,365, which are hereby incorporated by reference into this application in their entireties).

GreeneChip1.1, produced using mask-less printing technology (Agilent Technologies), has several advantages: (1) at 9066 viral targets it is more complex than the first generation array (represents a minimum of three 60 nucleotide oligonucleotide probes for each of the 1710 viruses in the ICTVdb); (2) oligonucleotides are synthesized in situ at right angle with respect to the planar surface to allow optimal exposure for hybridization; (3) the fidelity and reproducibility of spotting density is markedly improved (see FIG. 1); (4) GreeneChips are produced in batches of 20 to facilitate modification to include new sequences (larger runs can be conducted with the pin-driven spotting strategy); (5) human gene sequences have been introduced to allow assessment of host response; (6) implementation of an Agilent scanner allows automatic adjustment of the focal plane for improved resolution.

Table 7 below compares the viral array developed by DeRisi and colleagues (Wang, D. et al., 2003, “Viral discovery and sequence recovery using DNA microarrays,” PLoS Biol 1:E2) with GreeneChip1.1. The two array systems differ substantively in design, complexity and coverage of vertebrate viruses.

TABLE 7
Viral Array Comparison

| | 1^{st} Generation DeRisi Array | GreeneChip1.1 |
|---|---|---|
| Coverage | All completely sequenced viruses (vertebrate, | All completely or partially sequenced vertebrate |
| | plant, and phage) | viruses |
| | 1582 complete viral genome sequences | 98,310 vertebrate viral sequences |
| | (November 2002) | |
| Probe selection | Each genome divided into overlapping 70mers | PFAM and multiple alignments |
| strategy | offset by 25 nt | (assures coverage and redundancy) |
| | Pairwise BLASTN search between each 70mer | Conserved region at different levels (family, |
| | and family sequences | genus, species) |
| Number of probes | Five highest-ranking 70mers for each virus | Number of probes driven by biology (sequence |
| per target | (both polarities; hence 10 probes/virus) | variation, level of expression) |
| | | Minimum number to cover each sequence in |
| | | each taxa with < 4 mismatches |
| Array composition | 11,315 probes | 9066 probes (see list of viral and host targets |
| | 70mers | contained in CD-ROM Table Appendix filed |
| | ~1000 viruses | along with U.S. provisional patent application |
| | 661 vertebrate viruses | Ser. No. 60/861,365, which are hereby |
| | (39% of vertebrate viruses in the ICTVdB) | incorporated by reference into this application in |
| | | their entireties) |
| | | 60mers |
| | | 1710 viruses |
| | | 1710 vertebrate viruses |
| | | (100% of vertebrate viruses in ICTVdB) |

The DeRisi database is much more limited in scope for vertebrate viruses, is largely based on older reference strains for which complete genomic sequence is known, employs an algorithm that is less robust than the combination of PFAM and HHM, and is based on 70mer sequence targets as opposed to the 60mer sequences of the GreeneChip. The labeling protocol is also different. In summary, the GreeneChip system is broader, more specific, and more sensitive.

### Example 2

### Protocols for Microarrays of the Invention

The below protocols refer to the “GreeneChip,” but the methods of the invention can be generally applied to design oligonucleotide microarrays of the invention.

Method of Recovery of Viral Sequences From Greenechips

The specificity of hybridization signal can be tested by eluting and sequencing viral cDNA hybridized to arrays. In experiments using WNV, SARS, and Sindbis isolates, cDNAs ranging from 200 to 1000 nucleotide were obtained. GreeneChips can display a minimum of 3 or more probes representing different genomic regions for each virus; thus, this method allows rapid sequence characterization and phylogenetic analysis. The protocol is straightforward and does not require complex equipment; it can be readily implemented in clinical or field laboratories. A silicon gasket can be applied to the slide to define a well over the array. 200 ml of water is placed in the well at 65° C. for 10 min. The water containing the eluted cDNA is removed. 5 ml is used as template for PCR amplification with the specific amplification primer used to generate the hybridized product (denaturation at 94° C.×1 min, annealing 55° C.×1 min, extension 72° C.×90 sec, 35 cycles). Products can be ligated into a plasmid vector (pCR2.1-TOPO TA, Invitrogen) and used to transform competent E. coli. White colonies can be screened by direct colony PCR and dideoxy sequencing.

A Method of Viral Microchip Sample Preparation Hybridization and Labeling

Results can be compared with phenol based extraction systems (Tri-Reagent TR, tissues; Tri-Reagent LS, fluids), silica binding cartridges (Qiagen kits), and magnetic silica binding solutions (MiniMag/NucliSense systems). Performance of samples stored for delayed processing (RNAlater, Ambion) can also be tested. Negative controls can include relevant specimen types obtained from healthy volunteers, including blood, urine, sputum, and feces.

Extracts from previously diagnosed samples, or derived from experimental infections, can be quantitated by real time PCR prior to array analysis to confirm integrity of the material and to assess detection sensitivity. Unknowns that score negative on the respective specialized sub-array can be subjected to GreeneChip1.1 analysis.

Sensitivity is important to implementation of arrays with clinical materials. It can be important to establish a robust universal amplification method. Efficiency of individual steps of the protocol can be monitored and optimized using spiked samples and real time PCR.

RNA can be extracted from an infected cell using TriReagent (Molecular Resource Center). Following DNAse treatment and DNAse neutralization to eliminate host DNA, viral RNA can be enriched by column chromatographic subtraction of 28S and 18S rRNA. First-strand reverse transcription can be initiated with a random octamer linked to a specific primer sequence (5′ GTTTCCCAGTAGGTCTCNNNNNN) (SEQ ID NO: 1). After RNase H digestion, cDNA can be amplified using a 1:9 mixture of the above primer and a primer targeting the specific primer sequence (5′ CGCCGTTTCCCAGTAGGTCTC) (SEQ ID NO: 2). Initial PCR amplification cycles can be performed at a low annealing temperature (35° C.); subsequent cycles use a stringent annealing temperature (55° C.) to favor priming through the specific sequence.

Products of this first PCR can be then amplified in a second ‘labeling’ PCR using the specific primer sequence that is linked to a capture sequence for 3DNA dendrimers containing more than 300 fluorescent reporter molecules (Genisphere Inc.). The PCR product can be denatured in hybridization buffer (final concentration: 0.25M NaPO4, 0.5% SDS, 1 mM EDTA, 1×SSC, 2×Denhardt's Solution; pH 7) and added to GreeneChips for hybridization at 65° C. for 16 hours on a rotating platform. Following washes, a second hybridization step can be performed to add Cy3-labeled dendrimers (Genisphere, Hartfield, Pa.). GreeneChips can be incubated with the dendrimer hybridization mix at 65° C. for 1 hour, washed, dried, imaged using an Agilent DNA microarray scanner, and analyzed using Agilent Feature Extractor software.

The use of dendrimers can provide a 100× gain in sensitivity over microarray labeling methods where reporter molecules are directly incorporated into amplification products.

In concert, these modifications can have increased sensitivity from 1,000,000 RNA molecules (standard DOP-PCR protocol) to 1000 RNA molecules (GreeneChip protocol presented above) and can be sufficient to allow one to detect any of 1710 known vertebrate viruses or related viruses with the sensitivity required for clinical and surveillance applications.

Method of Optimizing Assay Performance Using Infected Culture Extracts

Target genes can be cloned into transcription vectors pGEM-Teasy (Promega) or pCR2.1-TOPO (Invitrogen) by conventional RT-PCR cloning methods. Quantitated plasmid standards can be used in initial assay establishment. Thereafter, RNA transcripts generated by in vitro transcription, quantitated and diluted in a background of random human RNA (representing brain, liver, spleen, lung and placenta in equal proportions) can be employed to establish sensitivity and specificity parameters.

One representative isolate for each viral select agent/gene and one isolate of each influenza H and N subtype can be used during initial assay establishment. Calibration reagents can be components of kits distributed to network laboratories and ultimately to customers. Three types of calibration reagents can be provided: 1) cloned target genes for performance tests, 2) tissue culture extracts calibrated by real time PCR for sensitivity assessment, and 3) a synthetic RNA target to be spiked in each sample prior to the reverses transcription as an internal positive control; a matching probe for this internal control is printed on the arrays.

Cell culture extracts of authentic pathogens can be used to optimize performance of RT transcription reactions, and to confirm detection of DNA virus (Poxyiridae) through its mRNA transcripts. RNA from tissue culture supernatant virus and infected cell layer can be extracted for RNA using commercial extraction kits and then treated with DNase.

DNase treatment can enhance the sensitivity of GreeneChip1.1. The efficiency of different RT enzymes will be compared by using real time PCR quantitation of cDNA produced from identical aliquots of the same sample.

Array Analysis Algorithm/Software for Automated Evaluation of Hybridization Results

A software tool can be tailored to analyze the pattern of positive signals (probes with fluorescence above the threshold level, defined as the average background plus three standard deviations). This software can eliminate signals from internal controls and position markers. Based on the taxonomic information implemented in the integrated Greene database, the software can analyze the detected hybridization pattern and calculate a ranked probability for that pattern to match a given viral family, genus and species.

Method & Algorithm/Software for Database Quality Control

Random subsets selected from a total of 432 vertebrate viruses can be purchased from the American Type Culture Collection (ATCC). Successful hybridization with a subset of 30 viruses selected at random from the 432 available isolates would yield a positive predictive value of 1.0.

If appropriate probes yield signal, provide an anticipated outcome and score as positive, then no further action may be needed.

If none or only a subset of appropriate probes yield signal, the outcome can relate to technical difficulty (no signal) or relative abundance of target. To confirm the presence of target template by quantitative PCR, hybridization can be repeated. If the pattern consistent with transcription gradient it can be scored as positive and no further action may needed. If probes fail, the probes can be redesigned.

If appropriate probes (or a subset thereof) yield signal but if an inappropriate probe also yields signal, they can be scored as positive and assessed for sequence homology. If there is no homology, then the probe can be replaced. If there is homology present, it can be recorded in the assay software as potential cross-hybridizing target and be considered for probe replacement if appropriate as it may serve as surrogate marker.

### Example 3

### Maintenance, Updates, Testing and Software of a Vertebrate Viral Database

The software can be designed to establish, implement and validate bioinformatics tools and databases to support microarray design and updates.

Design and Implement Software for Extracting Viral Sequence Updates

The rapid evolution of pathogens, particularly RNA viruses such as influenza, may compromise detection even by oligonucleotide microarray technology. Thus, the methods of the invention allow a continuous updating of databases and oligonucleotide sets. A key advantage of the Agilent mask-less printing technology is that GreeneChips can be rapidly modified at no additional cost. Thus, if needed, new versions of the GreeneChip can be introduced on a monthly basis.

Integration of Public and Proprietary Sequence Data to Create a Comprehensive Viral Database

Access to viral sequences that are neither published nor deposited in public databases can provide a more comprehensive database. For example, as the first installment from proprietary collections, the complete genome sequence of the following select agents were obtained and added to the vertebrate viral database: Crimean-Congo hemorrhagic fever virus, strains AP92, ArD8194, ArD15786, ArD39554, C-68031, Drosdov, Kashmanov, Oman, SPU97/85, SPU103/87, SPU415/85, Turkey200310849, UG30010; Rift Valley fever virus, strains ZH-501, ZM-657, ZC-3349, ZSS-6365, ZS-501-777, ZH-501-TI, 763/70 Rhodesia, 2250/74 Rhodesia, 2269/74 Rhodesia, 1260/78 Rhodesia, Kenya 98000523, Saudi 10911, Entebbe, Os-1, SA-75, Smithburn, Kenya56, Kenya57, MP12, Clone 13, CAR R1662, AnK6087, ANK3837, 73HB1449, MgHb24; Marburg virus, strains Ravn, DRC Nga, DRC Aru, DRC Dra, Angola05, and Voege67. These additions to the database can enhance the accuracy of initial probe design, and, via updates, ensure that the arrays remain current. In this context, it is noteworthy that while GenBank contains complete genome sequence for 2 strains of Marburg virus, the expanded database represents complete sequences for 15 strains of Marburg virus; similarly, while GenBank contains complete genome sequence for 2 strains of Crimean-Congo hemorrhagic fever virus (CCHFV), the expanded database represents complete sequences for 15 strains of CCHFV.

Updating Script

A script can be designed that will query GenBank/EMBL, SwissProt/UniProt, ISD and the CDC databases for new sequence entries at monthly intervals. New sequence entries can be curated for relevance (vertebrate viruses). This process can identify, for example, four types of new entries: (1) new sequence for a known viral species in a region represented on the array; (2) new sequence for a known viral species in a region not represented on the array; (3) new sequence for a previously uncharacterized viral species in a region represented on the array; and (4) new sequence for a previously uncharacterized viral species in a region not represented on the array.

(1) New sequence for a known viral species in a region represented on the array:

The new sequence entry can be compared to probes on the existing array. No action will be required if the sequence is covered by at least one existing probe with less than 4 nucleotide mismatches. New probes can be added if the coverage is inadequate using the existing probe set.

(2) New sequence for a known viral species in a region not represented on the array: The new sequence entry can be used to query the vertebrate viral database. If there is no related entry, or if homology to existing sequences is less than 90%, a new probe can be created. Without being bound by theory, the reasoning here is that divergence in one genome region not represented on the array may predict divergence in another region that is represented on the array.

(3) New sequence for a previously uncharacterized viral species in a region represented on the array: The new sequence entry can be compared to probes on the existing array. No action will be required if the sequence is covered by at least one existing probe with less than 4 nucleotide mismatches. New probes can be added if coverage is inadequate using the existing probe set.

(4) New sequence for a previously uncharacterized viral species in a region not represented on the array: The new sequence entry can be used to query the vertebrate viral database. If there is no related entry, or if homology to existing sequences is less than 90%, a new probe can be created. Without being bound by theory, the reasoning here is that divergence in one genome region not represented on the array may predict divergence in another region that is represented on the array.

Maintain and Curate the Updated Integrated an ICTV/NCBI Virus Database (Vertebrate Viral Database; Greene Viral Database)

Databases evolve and taxonomic nomenclature and protein domain assignments may change as a result of the accumulation of new sequences and insights into protein structure and function. To address this, at 6-month intervals relevant information can be downloaded from GenBank/EMBL, SwissProt/UniProt, ISD, CDC databases, ICTVdb, and PFAM. The downloaded information can be placed into a temporary integrated database and compared to the existing database.

Validate Database by Testing Performance of GreeneChip1.1 Using a Random Sample of Viral Targets

Logistical constraints make it impractical to validate the hybridization of 1710 vertebrate viruses, their variants, and subtypes. Therefore, validation of the oligonucleotide selection process and the resulting GreeneChip1.1 through a random sampling approach can be conducted.

The random sampling approach can be based on sampling theory, which predicts that a “stable system of chance” will behave according to intrinsic patterns that can be both anticipated and measured. Variation within each predictable pattern is inevitable. Variation outside this pattern can be detected with statistical quality control measurements (Grant, E., and R. Leavenworth, 1980, Statistical Quality Control, McGraw Hill, 5th Ed.). Results of hybridization experiments with West Nile virus, SARS coronavirus, and Ebola virus have been consistent. Thus, one can estimate the confidence intervals for sensitivity and specificity of the microarray for each target agent by randomly sampling the total number of possible experiments (i.e., the number of different organism species in the database, here, 1710 vertebrate viruses).

In a group of N potential experiments (e.g., up to 1710 species), from which we select a sample of size n (e.g., 30 species), the probability of measuring x success from a total of k possible successes (value to be predicted) follows a hypergeometric distribution. The “sampling experiment” is then known as a hypergeometric experiment (Johnson, N. et al., 1993. Univariate Discrete Distributions, 2nd Ed. John Wiley and Sons, Inc.). It follows from the previous description that the failure rate of the experiment is n−x, and the failure rate of the GreeneChip that we are trying to estimate with the experiment is equal to N−k.

Formula 1 illustrates the calculation of the hypergeometric distribution. For proportions of n/N<10, one can estimate the hypergeometric distribution with the binomial distribution provided in Formula 2.

Formula 1: Hypergeometric distribution. The probability of x successes (true positive prediction of the species) in a random sample of size n is equal to the product of the possible combinations of successes,

$$( \frac{k}{x} ) ,$$

times the possible combinations of failing arrays,

$$( \frac{N - k}{n - x} ) ,$$

divided by the possible combinations of samples of size n that can be drawn from the total number of species (N) for which we have isolates available,

$$( \frac{N}{n} ) .$$

$$h ( x , N , n , k ) ≅ b ( x , n , \frac{k}{N} ) = ( \frac{n}{x} ) \cdot {[ \frac{k}{N} ]}^{x} \cdot {[ 1 - \frac{k}{N} ]}^{n - x} , \\ when n / N < 10$$

Formula 2: The binomial distribution as an approximation to the hypergeometric distribution. When n/N<10, the hypergeometric distribution can be estimated with the simpler binomial distribution. [k/N] is the positive predictive value and [1−k/N] is the negative predictive value.

Random subsets can be sampled and selected from a total of 432 vertebrate viruses that can be purchased from the American Type Culture Collection (ATCC). Successful hybridization with a subset of 30 viruses selected at random from the 432 available isolates yields a positive predictive value of 1.0 (Table 8 below). Table 8 shows an estimation of the confidence intervals for the total number of viruses likely to be detected by the GreeneChip, according to the number of viruses accurately detected in experiments using a sample of n=30 randomly selected vertebrate viruses.

TABLE 8
Estimation of Confidence Intervals

| | | | | | | | Total number of |
|---|---|---|---|---|---|---|---|
| | Observed | 5% conf | | 95% conf | 5% | 95% | potential viruses |
| Sample | successful | interval | Estimated | interval of | confidence | confidence | upon which the |
| size (n) | detections (x) | of PPV | PPV | PPV | interval | interval | prediction is based |
| 30 | 0 | 0.00 | 0.00 | 0.00 | 0 | 0 | 432 |
| 30 | 1 | 0.00 | 0.03 | 0.10 | 0 | 43 | 432 |
| 30 | 2 | 0.00 | 0.07 | 0.13 | 0 | 58 | 432 |
| 30 | 3 | 0.03 | 0.10 | 0.20 | 14 | 86 | 432 |
| 30 | 4 | 0.03 | 0.13 | 0.23 | 14 | 101 | 432 |
| 30 | 5 | 0.07 | 0.17 | 0.30 | 29 | 130 | 432 |
| 30 | 6 | 0.10 | 0.20 | 0.33 | 43 | 144 | 432 |
| 30 | 7 | 0.10 | 0.23 | 0.37 | 43 | 158 | 432 |
| 30 | 8 | 0.13 | 0.27 | 0.40 | 58 | 173 | 432 |
| 30 | 9 | 0.17 | 0.30 | 0.43 | 72 | 187 | 432 |
| 30 | 10 | 0.20 | 0.33 | 0.47 | 86 | 202 | 432 |
| 30 | 11 | 0.23 | 0.37 | 0.50 | 101 | 216 | 432 |
| 30 | 12 | 0.27 | 0.40 | 0.53 | 115 | 230 | 432 |
| 30 | 13 | 0.30 | 0.43 | 0.57 | 130 | 245 | 432 |
| 30 | 14 | 0.33 | 0.47 | 0.60 | 144 | 259 | 432 |
| 30 | 15 | 0.37 | 0.50 | 0.63 | 158 | 274 | 432 |
| 30 | 16 | 0.40 | 0.53 | 0.67 | 173 | 288 | 432 |
| 30 | 17 | 0.43 | 0.57 | 0.70 | 187 | 302 | 432 |
| 30 | 18 | 0.47 | 0.60 | 0.73 | 202 | 317 | 432 |
| 30 | 19 | 0.50 | 0.63 | 0.77 | 216 | 331 | 432 |
| 30 | 20 | 0.53 | 0.67 | 0.80 | 230 | 346 | 432 |
| 30 | 21 | 0.57 | 0.70 | 0.83 | 245 | 360 | 432 |
| 30 | 22 | 0.60 | 0.73 | 0.87 | 259 | 374 | 432 |
| 30 | 23 | 0.63 | 0.77 | 0.90 | 274 | 389 | 432 |
| 30 | 24 | 0.67 | 0.80 | 0.90 | 288 | 389 | 432 |
| 30 | 25 | 0.70 | 0.83 | 0.93 | 302 | 403 | 432 |
| 30 | 26 | 0.77 | 0.87 | 0.97 | 331 | 418 | 432 |
| 30 | 27 | 0.80 | 0.90 | 0.97 | 346 | 418 | 432 |
| 30 | 28 | 0.87 | 0.93 | 1.00 | 374 | 432 | 432 |
| 30 | 29 | 0.90 | 0.97 | 1.00 | 389 | 432 | 432 |
| 30 | 30 | 1.00 | 1.00 | 1.00 | 432 | 432 | 432 |

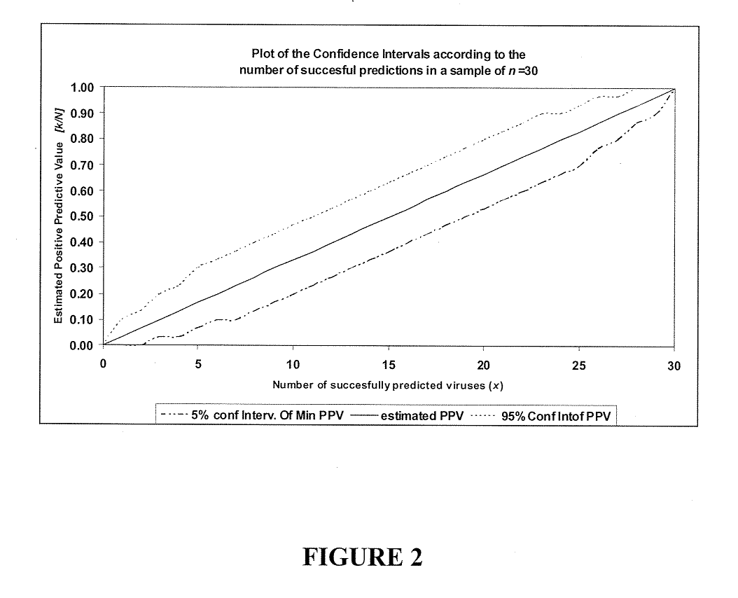

Table 8 and FIG. 2 show the confidence intervals for different success and failure rates, based on the binomial distribution, for a sample size of n=30 and a group of species of size N=432, according to every value of positive predictive values (PPV) by increments of 5% and for every PPV measured in the sample. In order to find the estimated PPV of the total set of 432, one identifies the PPV of the sample and looks up the corresponding estimated confidence interval.

$$h ( x , N , n , k ) = \frac{( \frac{k}{x} ) ( \frac{N - k}{n - x} )}{( \frac{N}{n} )} , x = 1 , 2 , 3 … n$$

Sampling Plan: 30 virus isolates will be randomly selected out of the possible 432 vertebrate viruses available for purchase from the ATCC. Following propagation in tissue culture, nucleic acid will be extracted and used as template for array hybridization. FIG. 2 illustrates the confidence intervals of the PPV according to the number of successful detections by the GreeneChip on a sample of 30 isolates. The sample size of 30 can be selected as a compromise between the increased precision in measurement of PPV that would be obtained with a larger sample and the resources and time available to conduct the experiments. As shown in Table 8, with a sample of 30 isolates, the confidence interval for a PPV of 90% is 80%-97%. In contrast, on a smaller sample of 10 isolates, the confidence interval for a PPV of 90% would broaden to 70%-99%. Using a sample of 60 isolates, the confidence interval for a PPV of 90% is 83%-97%, which is only slightly reduced compared to the 80-97% confidence interval associated with a PPV of 90% and a sample size of 30. Thus, the gain in precision by doubling the sample size from 30 to 60 is negligible, whereas the additional expense attendant to the greater number of experiments that would be required is substantial.

Accuracy Measurements

For each assay, there are three potential outcomes: (1) appropriate probes yield signal; (2) none or a subset of appropriate probes yield signal; (3) appropriate probes (or a subset thereof) yield signal but an inappropriate probe also yields signal.

(1) Appropriate probes yield signal: This can be an anticipated outcome and the result can be scored as positive. No further action is needed.

(2) None or only a subset of appropriate probes yield signal: The outcome can relate to a technical difficulty (no signal) or a relative abundance of a target. The presence of target template can be confirmed by quantitative PCR and hybridization can be repeated. If the pattern is consistent with transcription gradient, the results can be scored as positive. No further action needed. If probes fail, the probes can be redesigned.

(3) Appropriate probes (or a subset thereof) yield signal but an inappropriate probe also yields signal: The results can be scored as positive and assessed for sequence homology. If no homology is found, the probe can be replaced. If there is homology present, the assay can be recorded as a potential cross-hybridizing target and it can be determined if probe replacement is appropriate and may serve as surrogate marker. Inappropriate or false positive signals can also be detected using negative control RNA. Probes responsible for false positive signal can be redesigned.

Develop Array Analysis Software for Automated Evaluation of Hybridization Results

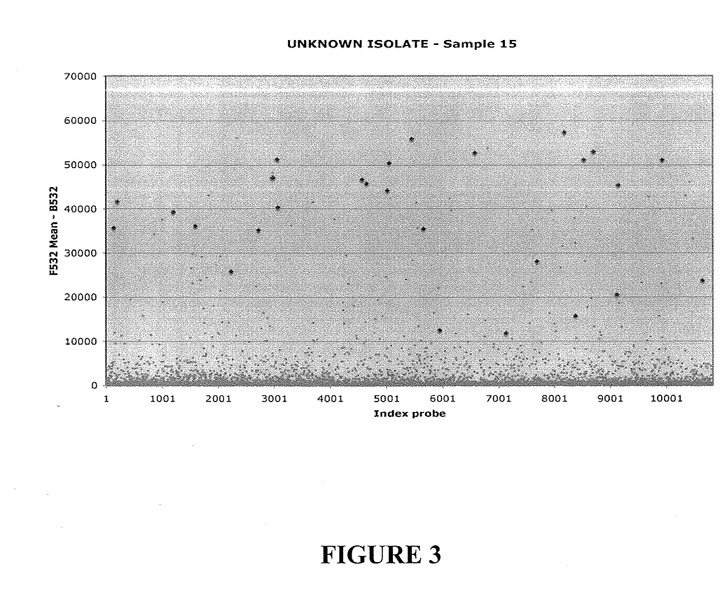

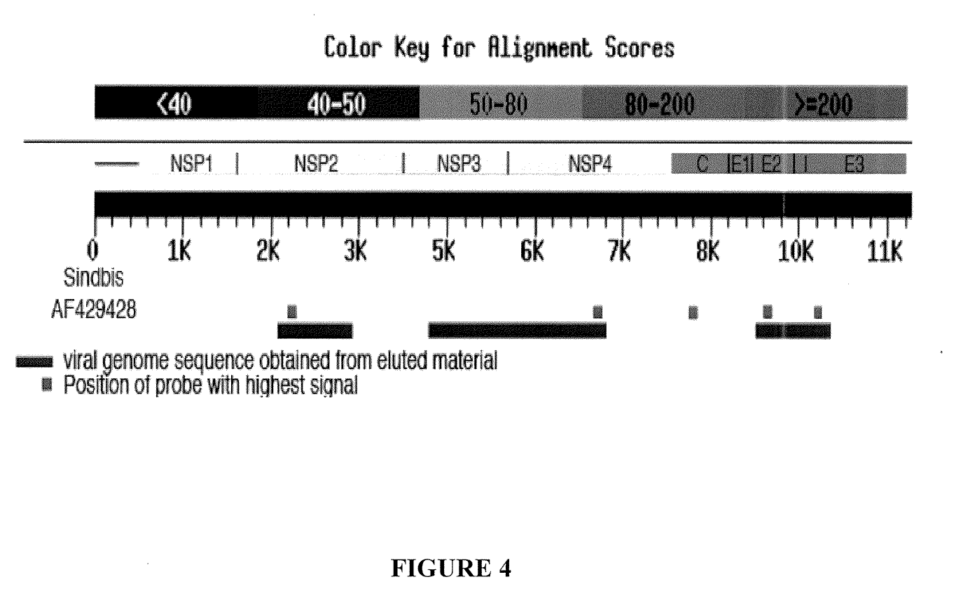

One goal of the present invention is to create user-friendly assays that can be deployed for hospital-based clinical laboratory and point-of-care differential diagnosis. Thus, bioinformatics tools are developed that allow automatic interpretation of the array results. GreeneChip microarray results are not amenable to analysis with tools used in expression profiling; expression-profiling results rely on the comparison of difference in signal intensity in two channels, while GreeneChip results follow primarily an on-off pattern. A software tool can be tailored to analyze the pattern of positive signals (probes with fluorescence above the threshold level, defined as the average background plus three standard deviations). This software will eliminate signals from internal controls and position markers. Based on the taxonomic information implemented in the integrated Greene database, the software can analyze the detected hybridization pattern and calculate a ranked probability for that pattern to match a given viral family, genus and species. FIG. 3 and Table 9 illustrate this process using a prototype script.

TABLE 9
Ranked Probability

| Significant hits | Genus | Corrected | Probes | Number |
|---|---|---|---|---|
| by genus | p-value | p-value | for genus | positive |
| Alphavirus | 1.30E−12 | 1.56E−10 | 369 | 28 |
| Significant hits | Species | Corrected | Probes | Number |
| by species | p-value | p-value | for species | positive |
| Sindbis virus | 4.76E−19 | 1.01E−28 | 42 | 16 |

### Example 4

### Creating Specialized Arrays for Detection and Speciation of Select Viral Agents and Influenza Viruses

Designing oligonucleotide targets: Currently there is no system that achieves comprehensive detection of H and N subtypes directly in clinical materials. Although other arrays for influenza are described, they require growth in culture prior to detection, target only influenza B, or lack resolution due to the use of 500 bp spotted cDNAs. The GreeneChip1.1 probes targeting influenza virus A, B, and C (˜600 probes) can be evaluated for coverage and differential detection of H and N subtypes. As required, additional probes can be designed to achieve complete coverage. The current platform employs 60mer oligonucleotides. Thus, while it has the potential to identify H and N serotypes and differentiate their major genotypes, it will not be sensitive to subtle differences in base composition. If this becomes a critical issue in diagnostics, sub-arrays can be created with shorter oligonucleotides (25mers) that can detect single base pair differences. However, it can be more practical to recover hybridized nucleic acids for direct sequencing.

The significance of deploying methods for speciation of influenza viruses is underscored by the genomic data obtained by the Influenza Genome Sequencing Project. The data explains why the 2003-04 annual influenza vaccine was not fully protective. During the 2002-03 season, different strains of the H3N2 virus underwent genetic mixing. The resulting strain emerged late in the season and was not addressed by the 2003-04 vaccine. Arrays for influenza virus speciation provide an inexpensive, high throughput solution for tracking strain evolution and facilitating vaccine design.

Probes of existing arrays can be refined to adapt to select agent detection and can also be designed to serve at the species level. Although it allows differentiation between Ebola Zaire, Sudan, Ivory Coast, and Reston viruses, it does not allow for distinction at the level of serotype, variant or strain. The latter can be important in epidemiological and forensic applications. New probes can be added to address differentiation below the species level where subtyping is informative, e.g., the four lineages of Eastern equine encephalitis virus (EEEV), or the serotypes of Venezuelan equine encephalitis virus (VEEV). Additional probes can include those for the detection of sequences important in pathogenesis that might be transferred naturally or deliberately into less pathogenic viruses. Examples include the Ebola virus G, p24, and p35 proteins, which have been implicated in immunosuppression and/or vasculopathy. In other cases such as poxviruses, where genome size and multitude of pathogenicity markers can prevent comprehensive coverage, at least 8 relevant genome regions can be targeted to better recognize chimeric genomes, and to ascertain differentiation of Variola major/minor from other poxviruses on the basis of redundant specific markers. Additionally, the set of probes can be supplemented with probes that identify non-select agents relevant to differential diagnosis because they may cause signs and symptoms that mimic those due to infection with select agents.

New probes, for updating probe sets and for creating arrays for biodefense and influenza epidemiology, can be designed. New or additional sequences can be mapped to PFAM domains and probes designed.

Although many applications can require slides that use up to 9,000-10,000 probes to represent the entire range of vertebrate viral targets, there can be instances where fewer probes are required because an objective is to differentiate only influenza viruses, or hemorrhagic fever viruses, or some other smaller taxon of viruses. A prototype has been produced wherein eight arrays can be simultaneously queried in independent experiments on a single slide (Agilent Technologies). To meet needs of this project and other high throughput applications, up to 24 independent 1500-probe arrays can be printed on individual slides (Agilent Technologies).

### Example 5

### Optimizing and Testing Assay Performance

Optimize Assay Performance Using Infected Culture Extracts

Target genes can be cloned into transcription vectors pGEM-Teasy (Promega) or pCR2.1-TOPO (Invitrogen) by conventional RT-PCR cloning methods. Quantitated plasmid standards can be used in initial assay establishment. Thereafter, RNA transcripts generated by in vitro transcription, quantitated and diluted in a background of random human RNA (representing brain, liver, spleen, lung and placenta in equal proportions) can be employed to establish sensitivity and specificity parameters. One representative isolate for each viral select agent/gene and one isolate of each influenza H and N subtype can be used during initial assay establishment.

Calibration reagents can be components of kits distributed to network laboratories and to customers. Three types of calibration reagents, for example, can be provided: 1) cloned target genes for performance tests, 2) tissue culture extracts calibrated by real time PCR for sensitivity assessment, and 3) a synthetic RNA target to be spiked in each sample prior to the reverses transcription as an internal positive control; a matching probe for this internal control is printed on the arrays.

Cell culture extracts of authentic pathogens can be used to optimize performance of RT transcription reactions, and to confirm detection of DNA virus (Poxyiridae) through its mRNA transcripts. RNA from tissue culture supernatant virus and infected cell layer can be extracted for RNA using commercial extraction kits and then treated with DNase. DNase treatment enhances the sensitivity of GreeneChip1.1. The efficiency of different RT enzymes will be compared via real time PCR quantitation of cDNA produced from identical aliquots of the same sample.

The amplification protocol for GreeneChip1.1 employs a random octamer-driven priming approach for reverse transcription followed by a random PCR amplification of cDNA. For the more specific sub-arrays targeting viral select agents or influenza viruses, one can examine whether alternative approaches can increase sensitivity beyond a threshold of 10³ copies/assay (see Table 10 below). In case of influenza, one can test the use of universal primer sequences that target the conserved terminal sequences of influenza A virus segments 4 (HA) and 6 (NA), or the respective segments of influenza B and C. Many of the viruses to targeted in select agent sub-arrays have conserved terminal sequences. Thus, it is possible to enhance sensitivity by using degenerate primer pools instead of random primers. Enrichment of relevant sequences can be obtained using pools comprising 30 to 60 degenerate primer pairs whether or not specific amplification is required. The value of alternative amplification strategies will be tested using one isolate representative of each select virus species and each influenza H and N serotype.

TABLE 10
Quantitative analysis of WNV detection on GreeneChip1.1 by real time RT-PCR.

| Target | Treatment | NA^{a} | RT^{b} | R-PCR^{c} | L-PCR^{d} | Hybridization |
|---|---|---|---|---|---|---|
| WNV | No DNase | 1.0.E+08 | 1.0.E+08 | 9.9.E+09 | 1.0.E+10 | Positive |
| | treatement | 1.0.E+07 | 1.0.E+07 | 8.0.E+09 | 1.0.E+10 | Positive |
| | | 1.0.E+06 | 1.0.E+06 | 7.3.E+10 | 1.0.E+10 | Positive |
| | | 1.0.E+05 | 1.0.E+05 | 8.0.E+10 | 1.0.E+10 | Positive |
| | | 1.0.E+04 | 1.0.E+04 | 1.0.E+03 | 1.0.E+05 | Negative |
| | | 1.0.E+03 | 1.0.E+03 | 1.5.E+04 | 8.0.E+03 | Negative |
| | | 1.0.E+02 | 1.0.E+02 | 1.1.E+01 | 8.0.E+00 | Negative |
| WNV | DNase | 1.0.E+08 | 2.0.E+08 | 1.2.E+10 | 1.5.E+11 | Positive |
| | treatement | 1.0.E+07 | 6.0.E+06 | 8.0.E+09 | 1.5.E+10 | Positive |
| | | 1.0.E+06 | 7.4.E+04 | 7.5.E+09 | 1.7.E+10 | Positive |
| | | 1.0.E+05 | 6.3.E+04 | 9.0.E+10 | 8.0.E+09 | Positive |
| | | 1.0.E+04 | 7.0.E+03 | 9.0.E+10 | 9.5.E+09 | Positive |
| | | 1.0.E+03 | 6.0.E+02 | 6.0.E+09 | 1.2.E+09 | Positive |
| | | 1.0.E+02 | 5.0.E+01 | 1.0.E+04 | 3.0.E+03 | Negative |

^{a}RNA, input copy number determined by real time PCR;
^{b}RT, copy number after reverse trancription;
^{c}R-PCR, copy number after random PCR;
^{d}L-PCR, copy number after labeling PCR.

For select agents, testing can be done on isolates available through the CDC SPB virus collection that differ genetically in the regions targeted by the oligonucleotide probes designed by the methods of the invention. Additional isolates can be added from other collections in the unlikely event they are not represented in the CDC SPB inventory. For influenza A viruses, testing can begin on one representative of each serotype. Thereafter, testing can be conducted on 400 random isolates collected over the past 57 years in North America, Oceania, Asia, and Europe.

Testing of Arrays on Influenza Virus Samples from New York State

Influenza virus isolates from the last 13 seasons can be retrieved, and a total of 130 isolates can be tested and analyzed. The total genomic sequence data for the isolates is available on GenBank. For each of the 13 seasons, 1992 through 2005, 10 isolates will be selected with a view to covering strains of influenza A and B that were detected in New York State during that season. These 130 isolates will be extracted, amplified, and tested on the microarray to confirm its ability to detect strains from the last 13 years.

Influenza Isolates from Australia

A total of 40 Australian isolates will be selected for analysis on the microarray to determine that it will also detect a range of strains from across several years at a distant geographical location. It is estimated that approximately 10% of samples may need retesting by the current molecular methods used in the clinical laboratory (real-time RT-PCR for influenza, conventional PCR for subtype determination, and sequencing for strain analysis) to verify results.

Historical Isolates

A total of 40 historical samples will be selected for analysis in the current microarray study. These will be reconstituted and inoculated into both eggs and Rhesus Monkey Kidney cells, in order to ensure sufficient virus for analysis. Due to no information about the virulence of these isolates, and as some are H2N2, work with these viruses prior to and including nucleic acid extraction, will be performed under BSL-3 containment. Additionally, since no molecular analysis has been previously performed on these isolates, they will be tested by real-time RT-PCR for influenza, conventional PCR for subtype determination, and sequencing of HA and NA genes in both directions for strain analysis. The analysis of these samples will ensure the microarray's ability to detect available influenza strains that have circulated in the human population in New York State for the last 57 years.

Primary Clinical Samples

From this sample repository, 100 specimens from each of the four seasons from 2004-08 can be selected for testing on the new microarray. For each season, 75 samples will have tested positive for influenza virus by one or more of the methods currently available in the laboratory, and 25 will have tested negative for influenza, but will include samples that tested positive for other human respiratory pathogens. These samples will represent as much diversity as possible in terms of type, subtype, strain, patient age, and geographic location throughout the state. Approximately 10% of samples may need retesting by the current molecular methods used in the clinical laboratory (real-time RT-PCR for influenza, conventional PCR for subtype determination, and sequencing for strain analysis) to verify results. The testing of these samples can be used to ensure the ability of microarrays having oligonucleotide probes designed by the methods of the invention to detect recently and currently circulating strains directly from clinical samples, and compare the detection sensitivity to that of the other currently available diagnostic techniques.

### Example 6

### Rapid Multiplex Primer Design From Nucleic Acid Alignments

Polymerase Chain Reaction (PCR) is a fundamental molecular biology tool and has been widely applied in pathogen detection. Various programs exist to automatically generate primers, though few programs use multiple sequence alignments as input. The invention provides a new method for generating degenerate primers by tree building and application of a set covering algorithm. Primer pairs were generated for viral hemorrhagic fevers, and then tested against DNA standards for sensitivity and specificity in multiplex PCR. Experimental results confirmed the utility of this primer design algorithm in pathogen detection.

Polymerase chain reaction (PCR) is the most widely adopted method for clinical detection of pathogens due to its speed, sensitivity and specificity. Primers for pathogen detection are designed to amplify highly conserved coding regions, though these may fail to hybridize due to wobble in the third codon. This problem can be solved by synthesizing primers with degenerate positions which cover the possible variants of a target strand. This method has been successfully applied to both pathogen discovery and cloning of homologous genes.

There is a consensus for appropriate GC content, acceptable hairpin lengths, melting temperature and maximum homopolymeric runs for sequencing primers (Buck, G. A., et al., Biotechniques, 1999. 27(3): p. 528-36.). These parameters also form the minimum requirements for diagnostic PCRs. In clinical applications, 3′ mismatches, degeneracy and the ability to multiplex are further concerns for primer design. The 3′ terminal base of the primer must exactly complement the target template because a polymerase will rarely extend a primer such a mismatch (Kuppuswamy, M. N., et al., Proc Natl Acad Sci USA, 1991. 88(4): p. 1143-7). This characteristic is often used for single nucleotide polymorphism (SNP) genotyping (Ross, P., et al., Nat Biotechnol, 1998. 16(13): p. 1347-51). Mismatches generally destabilize the binding of the primer to the template. If primers must have mismatches to their templates, mismatches should be closer to the 5′ end to keep the extension efficiency high.

Degeneracy is a critical factor in the sensitivity of PCR. A primer which is highly degenerate will have few species that are an exact match to the template. In the early rounds of PCR, the most homologous primers will likely be incorporated into amplicons, thus exhausting the exactly matching primers. Whether the reaction proceeds far enough to generate a detectable amount of amplicon is dependent on the similarity of the remaining primers in the solution. Some closely related primers may be able to amplify the products further, bringing the reaction to plateau even though they are not exact matches. Predicting the kinetics of this effect can be difficult because it is a function of the similarity of the primers, the concentration of the initial successful amplicons and the stringency of the reaction. One strategy is to design primers with the least number of degeneracies. If this is not possible, a cycling strategy like touchdown PCR can be used to progressively lower stringency. This will favor exact matches in initial cycles, but allow mismatching primers to continue the amplification in later cycles.

Primer Design Software

Current primer design programs implement a heuristic to find short DNA sequences which can be used for PCR. The heuristic is a scoring function based on parameters for successful primers which have been experimentally determined. Many tools exist for design of non-degenerate primers, the two most popular are Primer3 (Rozen, S, and H. Skaletsky, Methods Mol Biol, 2000. 132: p. 365-86) and Primer Express (www appliedbiosystems com).

To amplify a whole family of genes or agents, a consensus sequence can be generated from a multiple alignment and then used to synthesize primers. CODEHOP (COnsensus-DEgenerate Hybrid Oligonucleotide Primer) is a tool which uses multiple alignments of amino acids to create degenerate consensus primers in such a way (Rose, T. M., et al., Nucleic Acids Res, 2003. 31(13): p. 3763-6). Though very useful, the program can create primers which are highly degenerate, and may not be useful in diagnostic PCRs due to mispriming. Amplicon is a recently developed graphical tool for design of primers which identified potential sites for primers within a multiple alignment (Jarman, S. N., Amplicon: software for designing PCR primers on aligned DNA sequences. Bioinformatics, 2004). The user can input a primer sequence utilizing the suggested binding region, the program then scores the primer for melting temperature and secondary structure. The program does not provide a mechanism to minimize mismatches to the sequences, which is the most difficult part of degenerate primer design.

Computational Description of Primer Design Problem

Primer design can be considered a multi-criteria decision problem using a predefined scoring function to rank sequences. The upper and lower bounds of any parameter in the scoring function can be determined by the requirements and limitations of PCR. Parameters may have an ideal, a penalty for derivation and absolute requirements (Kampke, T. et al., Bioinformatics, 2001, 17(3): p. 214-25). The problem can be defined by: in a character string of length m, finding two substrings between length l to r which have a maximum score according to function S(p). The use of multiple alignments to create degenerate consensus primers introduces another level of complexity in the general problem. For a string a, with the alphabet [A, T, G, C] and length l find a string from the IUPAC degenerate alphabet (i.e., R=A or G) which covers the variability in the alignment with the minimum of degenerate positions. The search space of primers is bounded by the amount of degeneracy that can be tolerated in a PCR reaction and the number of mismatches a primer is allowed. When choosing a primer, the least degenerate is always desired above others with the same number of mismatches.

The Set Covering problem (SCP) has classically been used to describe an airline crew scheduling problem, where a group of crews must travel to a set of destinations with minimal cost. A brute force solution, which is an evaluation of combinations of sub-solutions, becomes computational intractable with only a few thousand possibilities. The SCP has been described as NP-hard (Johnson, M.R.G.a.D.S., Computers and Intractability: A Guide to the Theory of NP-Completeness. 1979, New York: W.H. Freeman), which means there is probably no polynomial time solution. A variety of approximation algorithms exist for solving the SCP, including Genetic Algorithms [Aickelin, U., An indirect genetic algorithm for set covering problems. Journal of the Operational Research Society, 2002. 53(10):p. 1118-1126.], Simulated Annealing [Sen, S. Minimal Cost Set Covering Using Probabilistic Methods. in ACM. 1993.], Linear Programming with branch and bound (Caprara, A., et al., Operations Research, 1999, 47(5): p. 730-743) and classical greedy methods (Slavik, P., 1997, Journal of Algorithms, 25(2): p. 237-254). For a review, see Caprara, A., et al., Annals of Operations Research, 2000, 98: p. 353-371). SCP has been applied in the biological literature, from HLA typing (Woodbury, M. A. et al., Comput Programs Biomed, 1979, 9(3): p. 263-73), identifying RNAi sequences (Zhao, W. et al., Artif Intell Med, 2005) and most recently oligonucleotides for identifying bacterial genomes (DasGupta, B., et al., Computational Science—Iccs, 2005, Pt 2, 2005. 3515: p. 1020-1028; Dasgupta, B., et al., Bioinformatics, 2005, 21(16): p. 3424-6).

In one embodiment, the invention provides a program that automatically determines optimum primer pairs from a group of nucleic acid multiple alignments. The program formulates the primer design problem as a SCP then leverages a fast, greedy algorithm to find the smallest set of primers that can amplify sequences in the alignment. The program was tested by designing primers to detect viruses which cause viral hemorrhagic fevers (VHF). The viruses have significant sequence variability due their RNA genomes and are medically important.

Materials and Methods

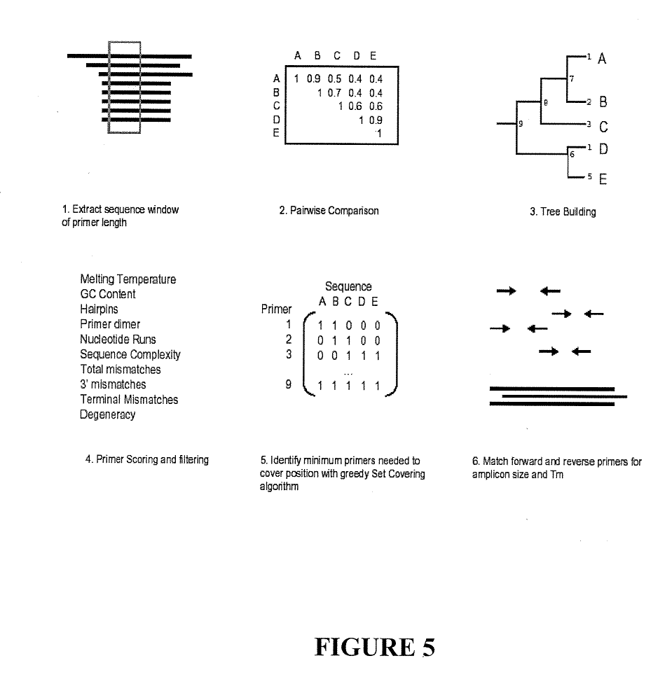

Algorithm and Implementation: The primer design algorithm consists of four parts, a tree building algorithm, a scoring function, the SCP approximation and primer pair matching (FIG. 5). Algorithms were programmed in Perl, using modules from the BioPerl distribution (http://www bioperl org) for sequence manipulation.

Pairwise Comparison and Tree Building: Sub-alignments of the user specified minimum to maximum primer length are extracted from the entire alignment and filtered for uniqueness. A pairwise comparison is used to generate a similarity matrix for each sub-alignment. The matrix is used to generate a phylogenetic tree, using a Hierarchical clustering algorithm based on Euclidean distance from the open source C Clustering Library (de Hoon, M. J. L., et al., Bioinformatics, 2004. 20(9): p. 1453-1454).

Scoring Function: A strict consensus at each node of the phylogenetic tree is computed then scored for user-specified parameters. First, primers are checked for the physical constraints like Tm, GC content, homopolymeric runs, hairpin/primer-dimer formation, and degeneracy. Primers outside acceptable cutoffs are removed from further consideration. Remaining primers are compared to sequences in the sub-alignment to determine if they are likely to hybridize and extend the template. In this phase of the scoring, total mismatches to templates, 3′ mismatches and terminal 3′ mismatches are identified for each primer (FIG. 5, step 4—primer scoring and filtering). The output of the mismatch function is a binary value, 1 means the primer can extend the sequence template, 0 means it will not. In computational terms, it is an integer matrix which represents a set to cover (sequences, as columns) and elements which accomplish the covering (primers, as rows), this matrix is illustrated in FIG. 5, step 5.

Set Covering Algorithm: The primer hybridization matrix is the input for the greedy SCP approximation algorithm. In this formulation of the SCP, the primer count is used as a cost function to optimize. The SCP algorithm is implemented in Perl, using the method described in [16]. The output of the algorithm is a minimal set of primers which can amplify sequences in the sub-alignment, if a set exists.

Primer Matching: The final stage is identifying primer pairs that form useful amplicons for PCR. The user can specify the minimum and maximum length amplicons, and the maximum difference in Tm between primer sets and pairs which pass the parameters will be returned in a tabular format. The software is licensed under the GNU GPL, and is free for non-commercial, academic use. A web version is available at http://www greeneidlab columbia edu/PrimerDesign.

Viral Hemorrhagic Fever (VHF) PCR: VHF sequences in various genes were extracted from NCBI GenBank. The viral targets for design included Ebola Zaire, Crimean Congo Hemorrhagic Fever (CCHV), Seoul Hantavirus, Kyasanur Forest virus and Rift Valley Fever virus. They were aligned using ClustalW (Nucleic Acids Res, 2003. 31(13): p. 3497-500), then submitted to the primer design program. The parameters used were: length, between 20 and 29 bp, Tm 50-65° C., GC 40-60%, maximum allowed hairpins of 8 bp, maximum continuous nucleotides of 4, maximum degeneracy 8, no mismatches allowed in 5 bp of 3′ end, maximum of 5 mismatches to the any template, and amplicon size of 60-150 bp. Finally, primer pairs should have melting temperature within 3 degrees of each other. Of the acceptable set, a portion was synthesized for testing.

Standards and Cycling Protocol for VHF PCR: To verify the sensitivity and specificity of the primers, DNA standards were made by overlapping PCR for the reference strains of VHF. Long 60 bp oligonucleotides with 20 bp overlapping segments were synthesized, annealed and amplified with High-Fidelity PCR Master kit (Roche, Indianapolis, Ind.). The amplicons were cloned into pGEM-T-Easy vector (Promega, Madison, Wis.) then sequenced to ensure no errors were introduced by the polymerase. Standards were generated by diluting linearized plasmid into 25 ng/ul Human Placental DNA (Sigma-Aldrich, St. Louis, Mo.).

The HotStartTaq polymerase Multiplex PCR kit (Qiagen, Hilden, Germany) was used to amplify the templates. Primers were used at a final concentration of 1.25 μM for singleplex and 0.5 μM each for multiplex with 0.5 mM Cl2Mg and 5 μM dNTP. The following cycling protocol used: an annealing step with a temperature reduction in 11° C. increments from 65° C. to 51° C. during the first 15 cycles, and then 35 cycles continuing with a cycling profile of 94° C. for 20 sec., 50° C. for 20 sec., and 72° C. for 30 sec. in a MJ PTC200 thermal cycler (MJ Research, Waltham, Mass., USA).

Results and Discussion

Viral Hemorrhagic Fever PCR: RNA viruses from various families are the agents responsible for hemorrhagic fevers, they include: Filoviridae (Ebola and Marburg viruses), Arenaviridae (Lassa virus, Junin virus, Machupo virus, Guanarito virus, and Sabia virus), Bunyaviridae (CCHF virus, Rift Valley fever virus (RVF), and Flaviviridae (Yellow fever, Kyasanur forest and Dengue viruses). The most striking symptom of these viruses is bleeding diathesis after infection. Other symptoms include fever, shock, headache, myalgia, and upper respiratory track complaints; most of which are not diagnostic of VHF infection. No cures exist, but ribavirin has been shown to be effective if given early after infection with Lassa virus, hantavirus, or CCHF virus.

Since VHF symptoms are nonspecific in the early phase of infection, clinicians need rapid differential diagnostics to control outbreaks. PCR is the method of choice, since it is rapid, flexible and can be deployed in the field. Designing PCR primer sets for a variety of pathogens which can be multiplexed is a daunting task, especially in quickly mutating RNA viruses. Primers were designed for five VHF pathogens to validate the SCP based algorithm, and to it can create high quality primers for multiplex. Nucleotide alignments were created for Ebola Zaire, Crimean Congo Hemorrhagic Fever virus (CCHV), Seoul Hantavirus, Kyasanur Forest virus and Rift Valley Fever virus. The alignments contained less than 30 sequences in most cases, and contained areas of high conservancy. Primers were designed using the parameters described in the methods; sequences are detailed in Table 11. Since the alignments were small (less than 30 sequences in most cases) the design time was negligible. Primer pairs conforming to the parameters were identified for viruses except CCHV. The forward primer has a very high Tm, but was the only choice without violating the terminal mismatch requirements.

TABLE 11
Viral Hemorrhagic Fever Primer Sequences

| | | | | | | |
|---|---|---|---|---|---|---|
| | Reference | | | | Tm | Amplicon |
| Virus | Strain | Gene | Direction | Sequence | ° C. | Length |

| | | | | | | |
|---|---|---|---|---|---|---|
| Ebola | NC_002549 | L | F | AACACCGGGTCTTAATTCTTATATCAA | 54 | 86 |
| Zaire | | Polymerase | | (SEQ ID NO: 3) | | |
| | | | R | GGTGGTAAAATTCCCATAGTAGTTCTTT | 55 | |
| | | | | (SEQ IDNO: 4) | | |
| CCHV | NC_005302 | Nucleocapsid | F | AGAACACGTGCCGCTTACGCCCA | 63 | 118 |
| | | | | (SEQ ID NO: 5) | 52- | |
| | | | R | CCATTCYTTYTTRAACTCYTCAAACCA | 56 | |
| | | | | (SEQ ID NO: 6) | | |
| Seoul | NC_005236 | Nucleocapsid | F | CAGGATTGCAGCAGGGAAGA | 55 | 67 |
| Hantavirus | | | | (SEQ ID NO: 7) | 53- | |
| | | | R | ATGATCACCAGGYTCTACCCC | 54 | |
| | | | | (SEQ ID NO: 8) | | |
| Kyasanur | AF013385 | NS5 | F | TGGAAGCCTGGCTGAAAGAG | 55 | 64 |
| Forest | | | | (SEQ ID NO: 9) | | |
| virus | | | R | TCATCCCCACTGACCAGCAT | 54 | |
| | | | | (SEQ ID NO: 10) | | |
| Rift | NC_002045 | NS | F | GGATTGACCTGTGCCTGTTGC | 55 | 108 |
| Valley | | | | (SEQ ID NO: 11) | | |
| Fever | | | R | GCATTAGAAATGTCCTCTTTTGCTGC | 58 | |
| Virus | | | | (SEQ ID NO: 12) | | |

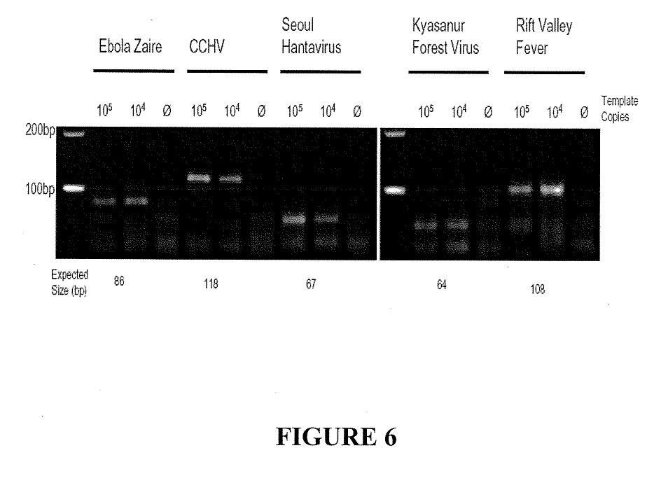

Using linearized DNA standards of the VHF reference strains, the sensitivity of the PCR primers were assayed in singleplex. Sequences were amplified, with the appropriate sized amplicons at a 50,000 copies. Further testing for the utility of primers in multiplex was carried out. Primer pairs for the VHFs were mixed together, to a final concentration of 0.5 μM/primer. DNA standards were tested separately by multiplex PCR. All VHFs were successfully detected at 50,000 copies, with minimal mis-priming to human DNA (FIG. 6). Analysis of the primer binding sites show they are in highly conserved regions, some with nearly 100% conservation.

The rapid and successful design of multiplex ready PCR primers is useful for pathogen detection. The design for this small set of viruses was completed in less than a day, and oligonucleotide synthesis could have been completed overnight. This method can be used in outbreak situations where a set of primers is needed for differential diagnosis.

Computational Complexity

The use of the greedy Set Covering algorithm guarantees a O log 2 nm (Slavik, P., Journal of Algorithms, 1997, 25(2): p. 237-254) which is quite good for a NP-Hard problem. Since the possible solution sets are strongly bounded by the requirements for PCR amplification, there is a high probability that the primer set found by the greedy algorithm will be the optimum set.

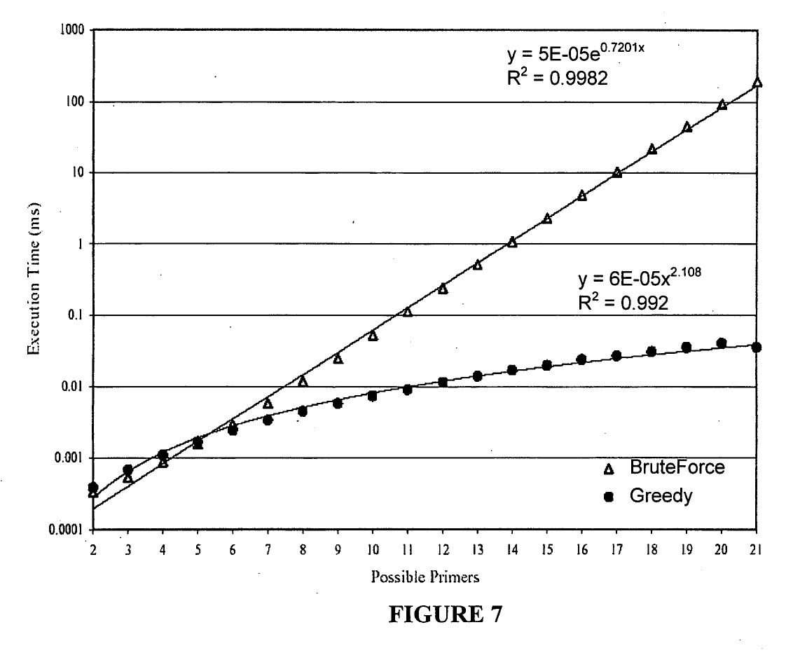

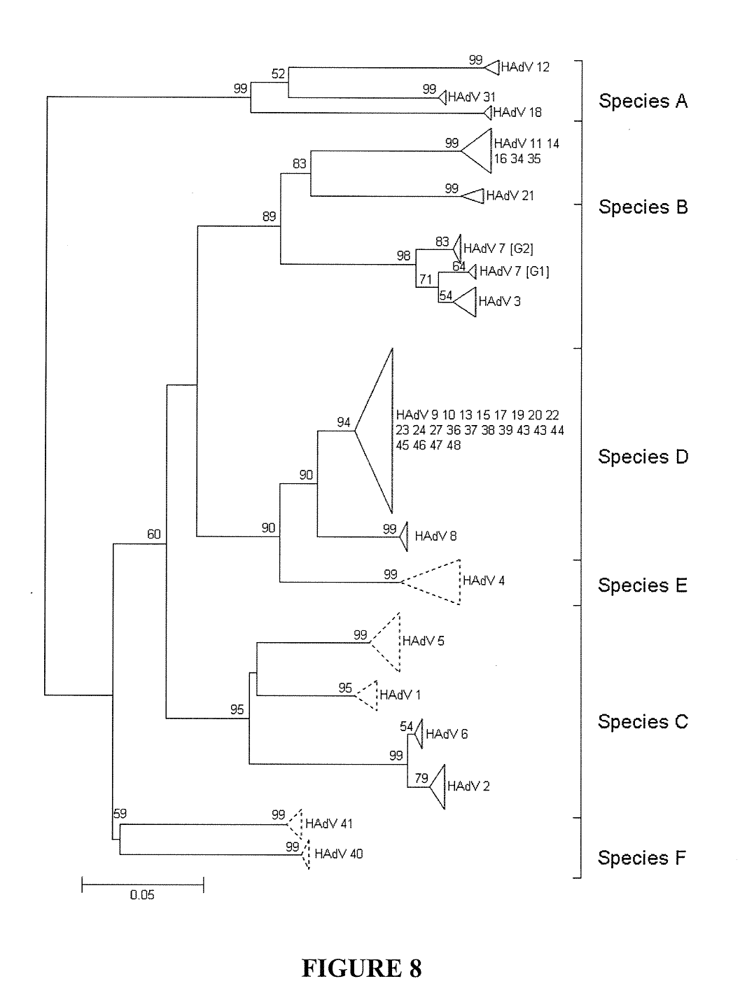

A comparison of the greedy heuristic to a brute force exact solution was carried out for a large alignment of the Influenza Hemagglutinin gene (HA5). The alignment was downloaded from the Influenza Sequence Database (flu.lanl.gov), and contains 550, 2 kb sequences. FIG. 7 shows the computational time for increasing number of possible primers. As expected, the time for the greedy implementation scales logarithmically, while the brute force approach is exponential. Even though the alignment had 550 sequences, the number of possible primers for a position did not exceed 21. This is due to the strict requirements on Tm, secondary structure and 3′ mismatches.

Interestingly, both algorithms returned exactly the same solution for all primer coverage problems analyzed. To further investigate this relationship, randomly generated coverage matrixes were created and solved by Greedy and Brute Force implementations. With this complex dataset, there were slight differences between the two. As expected, Brute Force had the better score when they differed. The underlying tree structure of the primers may be contributing to the success of the Greedy implementation. This relationship can be the topic of further investigation with other viral families or a mathematical approach.

Algorithm Optimizations

Three optimizations were implemented to improve the speed of the primer design program. First, the primer search space is restricted by only considering sequences which are consensuses to each branch of the phylogenetic tree. The maximum number of primers to consider is the number of nodes on the tree, or (n*2)−1. This is reduced to the number of primers passing the degeneracy, Tm, secondary structure and other requirements.

The second optimization is the use of a staged scoring function. The algorithm can be very computationally intensive due nearest-neighbor melting temperature or secondary structure prediction, which must be performed on each primer evaluated. In an actual implementation, the set of minimum requirements for a primer is very strict, leaving few possible pairs. Calculations which are inexpensive to perform can be very selective, for example: GC content, single nucleotide repeats and terminal mismatches to templates are lightweight pattern matches. To avoid excessive computational time, the scoring function postpones expensive calculations until the minimum requirements have been met.

Finally, only primers that cover a large number of sequences continue to the SCP solver. The initial step of the SCP is to identify subsets in the coverage matrix and exclude them. For example, if primer A covers sequences 1-5 and primer B covers 3-5, B is disregarded since it is a subset of A. The SCP is implemented using bitwise comparisons, so the set theory computations are done in machine language. This is significantly faster and uses less memory than creating the same matrix in a high level language like Perl.

Application to Pathogen Detection

This program was created to address the specific needs of virologists and bacteriologists in designing primers for pathogen detection. By design, the program provides primers which pass the user criteria. Although an “optimum” sequence could be estimated by a more complicated scoring mechanism, this would not reflect the reality of experimental PCR. Investigators who want to generate primer pairs with sensitivity in the 10-50 molecule range generally tune 5 or more potential pairs by modifying PCR conditions such as annealing temperature, MgCl₂ concentration, cycling time and number. Due to the significant validation time required for highly sensitive and specific primers, an automated design program which incorporates the parameters used by scientists provides a reliable starting point for experimentation.

This novel application of SCP significantly improves the specificity of primers in a complex alignment. A single, highly degenerate primer could be designed to cover two divergent groups. In practice, the PCR would be more successful if two different primers were designed targeting subgroup of sequences, resulting in primers with less degeneracy. The choice of where to split a single highly degenerate primer to cover subgroups is not obvious when designing primers by hand. One could use the phylogeny and split the alignment by clade, then identify consensus sequences for each group. Since phylogenetic trees are created with large regions, primer binding sites may not reproduce the same relationship. Calculating a similarity tree at each position of the alignment and using it to guide primer design is a simpler solution. The SCP based primer design program guarantees a near optimal result for this type of problem.

During the course of infection, viruses and bacteria exhibit high rates of mutation followed by purifying selection. This leads to a huge diversity of strains, which may vary in pathogenicity and hosts. VHF viruses were chosen as a test for the primer design methodology since they represent a medically important example of this diversity. The primer design program can be applied to any nucleic acid alignment. Immediate uses include identifying consensus primers for VHF families. A panel of PCRs could be used to find the animal reservoirs of VHFs and identify existing strains before they acquire the ability to infect humans.

The invention provides a primer design program which uses the well studied Set Covering Problem, coupled with a tree building approach to identify degenerate primer sets. The algorithm was tested with alignments of viral hemorrhagic fever pathogens, and successfully designed primers which were sensitive and specific. This method has wide applicability, for example, for diagnostic PCRs, for PCR cloning of entire families of genes, and for designing oligonucleotides for microarrays.

The algorithm can be further optimized by using modern linear programming methods to solve the SCP formulation. This would have the benefit of an exact solution, without the time required of the brute force method. Also, the program can be modified to take coding information into account. Many viral diagnostic PCRs target expressed genes, so sequence variation will be limited by selective pressure. Given a protein and nucleic acid a multiple alignment, the wobble codon can be identified. Enumerating conservative mutations and including them in the primer design process could be used to design primers which detect remote homologues.

### Example 7

### Adenovirus Typification by Oligonucleotide Microarray Hybridization

The invention provides a rapid, sensitive molecular system for human adenoviruses (HAdV) typing based on hybridization of hexon gene products amplified by PCR to an oligonucleotide array decorated with HAdV genetic targets. This system was validated with clinical isolates and is amenable to high throughput serotyping for epidemiological applications.

HAdVs are a significant cause of respiratory tract infections, some leading to fatal pneumonia or bronchiolitis in children or immunocompromised adults. They are also common agents of gastroenteritis, conjunctivitis, and keratoconjunctivitis, and infections on bone marrow transplant recipients. While there is no treatment for adenoviruses, virulence varies with strain. Thus, typing of adenovirus infections has clinical and epidemiological importance.

Adenoviruses, belonging to the family Adenoviridae, are non-enveloped, linear double-stranded DNA with genomes of 26-45 kb. The icosahedral capsid of the virus is composed of the hexon structural protein and the fiber protein, which mediates viral entry into hosts. HAdVs are divided into six species (A to F) based on molecular phylogeny, fiber protein variation, and biological activity. Traditional techniques of HAdV identification such as viral culture or sera neutralization, are being quickly supplanted by molecular methods for detection and serotyping. A variety of PCR based methods have been established including fluorescence based assays, singleplex PCR, multiplex PCR, and PCR in combination with restriction enzyme based typing. Molecular phylogenetic approaches are the most rigorous but require sequencing and may indicate different relationships as a function of the genomic region selected for analysis.

DNA microarrays, originally applied to host gene expression analysis, have recently been gaining favor as a tool for viral epidemiology. Viral families targeted for array based detection include herpes, retroviruses, human papilloma viruses, influenza virus, rotaviruses, and orthopoxviruses. An adenovirus typing array has been developed, based on regions in the hexon, fiber, and E1A genes, that can differentiate 5 of the 51 known serotypes. However, its utility in a clinical setting is limited since it requires three PCRs, followed by a hybridization step. In contrast, the invention provides a rapid typing method designed for implementation in a high throughput clinical setting.

A sensitive universal PCR was established that detects hexon gene sequences of known ADV in clinical samples. DNA standards were created for 47 different serotypes of ADV from viral stocks (ATCC, Manassas Va.) or cultured samples. Products were cloned into pGEM-T-Easy (Promega, Madison, Wis.) and sequenced to confirm fidelity. Linearized plasmid DNAs diluted into 25 ng/μl human placental DNA (Sigma-Aldrich, St. Louis, Mo.) were used as standards for optimizing PCR conditions. The PCR protocol of Avellon et al. (J Virol Methods, 2001, 92:113-20) was modified to obviate the need for nesting. PCR amplification was done using HotStart Taq PCR (Qiagen, Hilden, Germany), primers at 1.25 μM each, 2 mM MgCl₂, 0.2 mM each dNTP, and the following cycling protocol: 10 min. at 94° C., an annealing step with a temperature reduction in 1° C. increments from 65° C. to 51° C. during the first 15 cycles, and then continuing with a cycling profile of 94° C. for 20 sec., and 72° C. for 30 sec. in a MJ PTC200 thermal cycler (MJ Research, Waltham, Mass.).

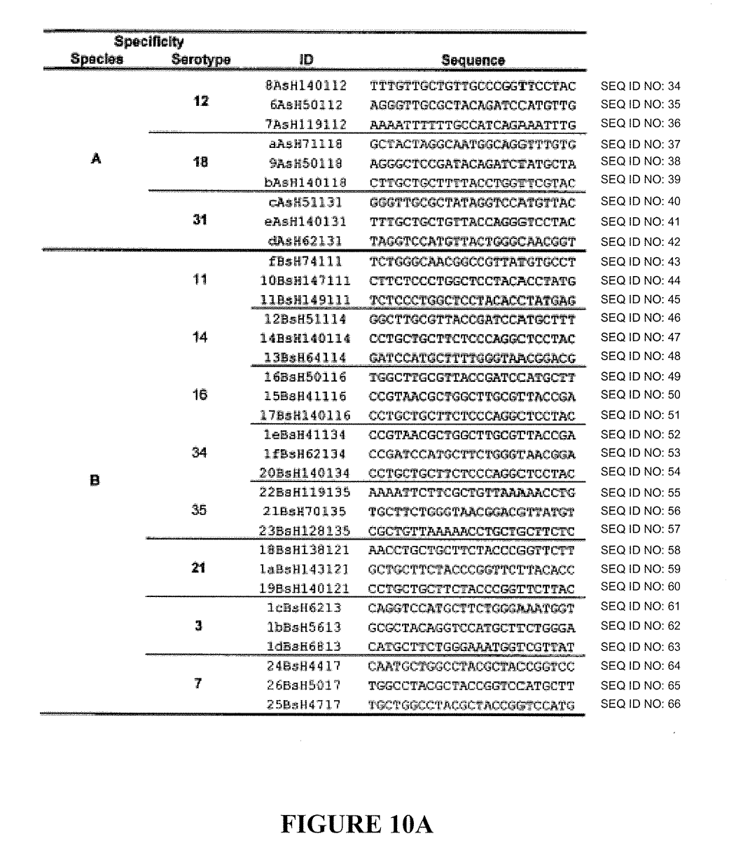

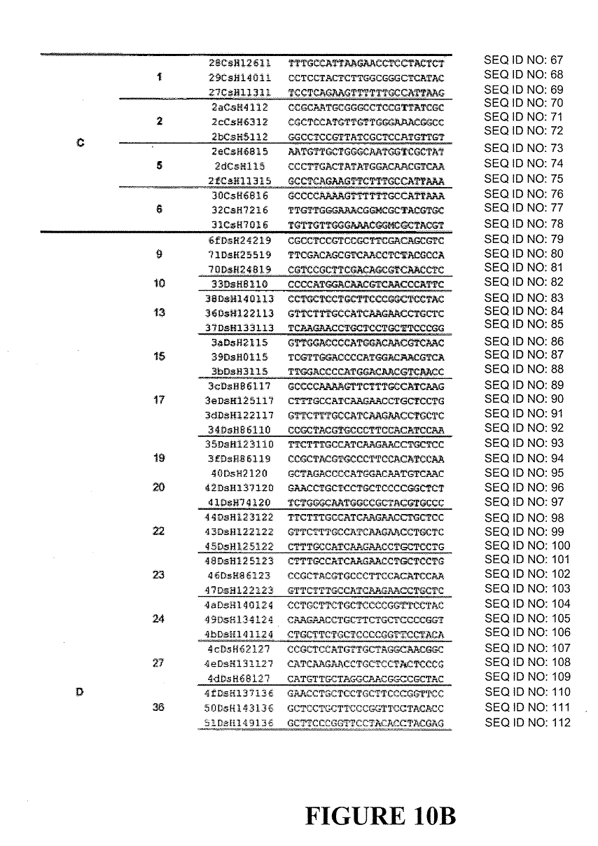

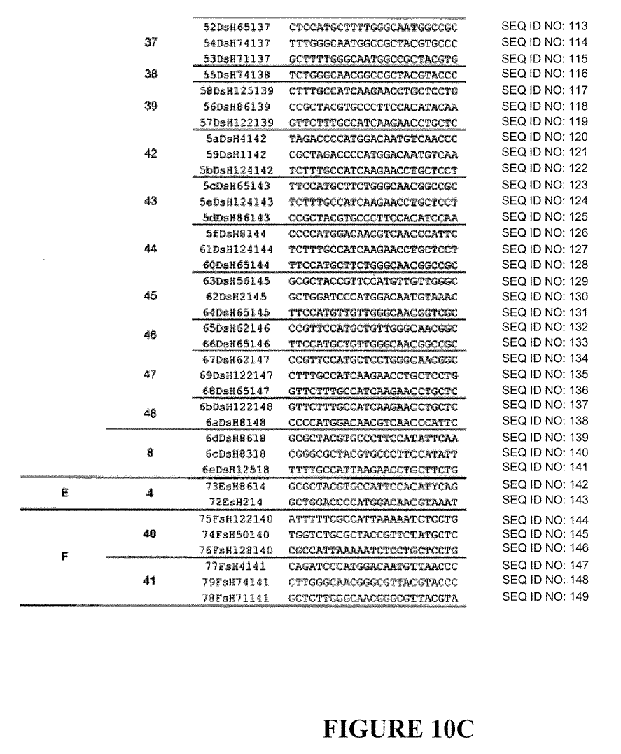

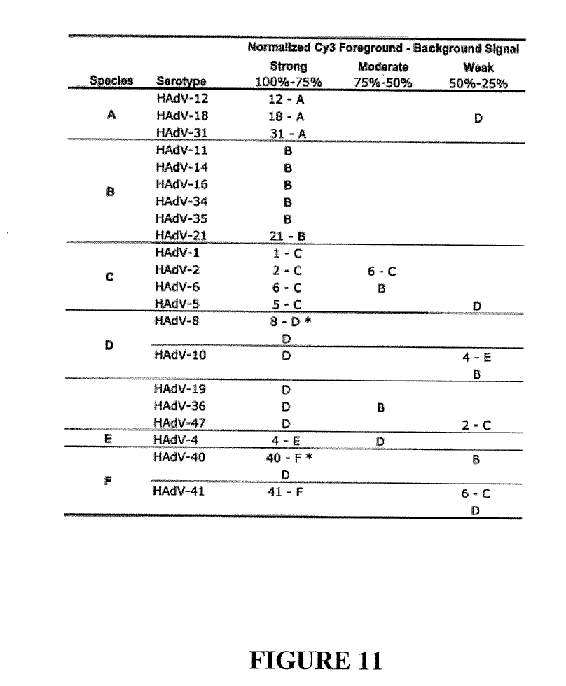

A database of hexon sequences was generated by extracting HAdV sequences in Genbank. The sequences were classified into serotypes using phylogenetic inference. Serotypes 12, 31, 18, 21, 7, 3, 8, 4, 5, 1, 6, 2, 40, 41 had unique sequence features which allowed specific oligonucleotide targets to be designed (FIG. 10). With the exception of HAdV-8 in species D and HAdV-3, -7, and -21 in species B, all species D or species B serotypes were unable to be resolved using the 460 bp hexon fragment.

A Perl based program was created to design 25 bp oligonucleotides for the array. Probes were selected to hybridize specifically to members of the target serogroup. 116 oligonucleotide targets were designed to cover 48 serotypes (FIG. 10). Pilot studies indicated that 6 mismatches were sufficient to preclude hybridization; thus, where feasible, serotype specific probes were designed to have at least 6 mismatches with other serotypes. Species D and species B probes were designed to minimize hybridizations with serotypes outside the species. Three different probes were designed for each serotype to minimize the potential of failure due to secondary structure, melting temperature, or high sequence divergence (FIG. 10A-C).

Target oligonucleotides were synthesized with a 5′ amino-link and spotted onto epoxy coated slides at 25 μM (MWG Biotech, High Point, N.C.). Each target was printed in triplicate to minimize the effect of technical artifacts. To enhance throughput and reduce reagent costs, the spots were arranged in 16 sub-arrays per slide. Sub-arrays were isolated by a silicone gasket to allow 16 independent hybridizations. Four slides could be locked into a microtiter plate adaptor tray to process 64 samples simultaneously (Grace BioLabs, Bend, Oreg.). A quality control oligonucleotide (GAAACTGTAGATTCTCCAAGATCCA) (SEQ ID NO: 13) was included in each target spot at 0.5 μM. This control target was hybridized with a fluorescently labeled complement as a probe to evaluate spot morphology and DNA density.

After initial PCR screening to confirm the presence of HAdV hexon sequences, the residual amplification product was subjected to asymmetric PCR to generate single stranded probes for hybridization. The reverse primer HEX2R used in the initial PCR was modified to incorporate a sequence for secondary hybridization to a fluorescently tagged 3DNA molecule during the asymmetric PCR (Genisphere, Hatfield Pa.). The modified reverse primer was used at a concentration of 100:1 relative to the 20 μM forward primer in a standard PCR using AmpliTaq polymerase (Applied Biosystems, Foster City, Calif.). The single stranded product was hybridized at 42° C. with a buffer containing 3×SSC, 5×Denhardt's, and 0.01% SDS for 1 hour. Slides were washed with 4×SSC for 5 minutes, dried, then 0.2 μl 3DNA-Cy3 and 1 μl of 1 μM Cy5 tagged quality control oligonucleotide were hybridized at 42° C. for 1 hour in the same buffer. Slides were washed with 4×SSC, dried, and then scanned on Axon GenePix 4000B Array Scanner (Union City, Calif.). Images were analyzed using GenePix Pro 5. Array spots with excessive background were excluded from the analysis. The foreground 532 nm Cy3 fluorescence minus background Cy3 fluorescence (F532-B532) was used to measure the spot intensity. To allow data comparison across experiments, the F532-B532 values were normalized as a percentage of the highest value.

Reference strains of HAdVs from each species were processed for hybridization to the array. Initially, spot fluorescence was normalized by DNA density and Z-Scores calculated for analysis. Since the array printing was highly uniform, this method proved needlessly complex. To simplify analysis, the normalized Cy3 fluorescence measurements were split into quartiles. Signals were above 75% of maximum were classified as “strong”; between 75% and 50% were “moderate” and 50% to 25% were “weak.”

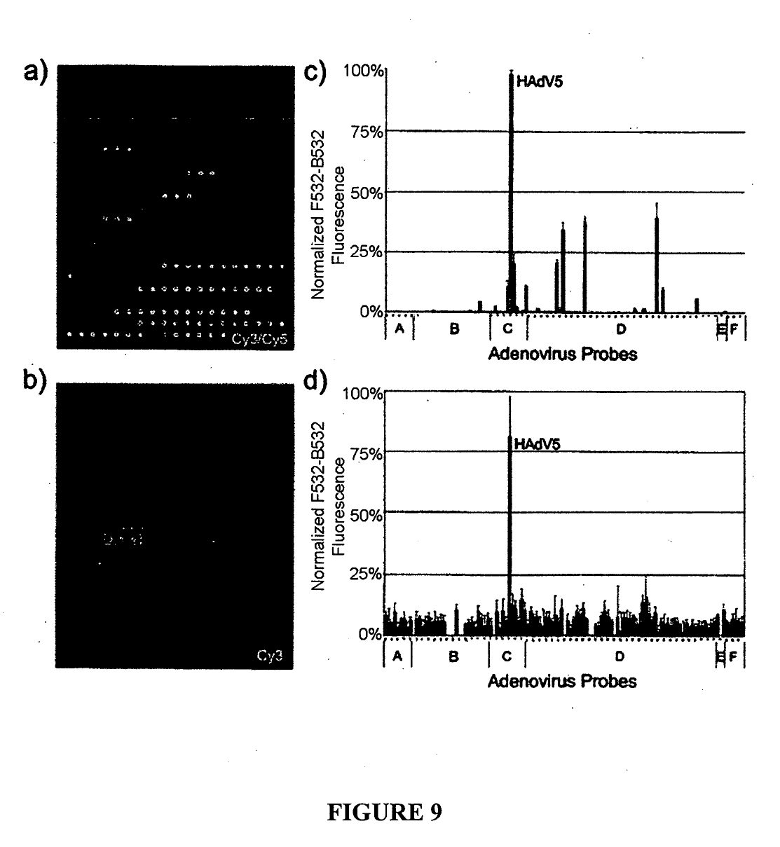

Probes designed for specific serotypes showed strong signal for their cognate targets (FIG. 9). FIG. 9 shows hybridization of HAdV-5 with a matched clinical sample of the same serotype (SO4367), illustrating the specificity of hybridization signal.

Within the panel of 47 HAdV probes, one instance was found where binding was promiscuous. One Species D probe generated high signal with a HAdV-40 (Species F) hexon product; however, higher signal for this product was obtained with serotype 40 probes. Thus, despite cross reactivity the array allowed accurate identification of serotype.

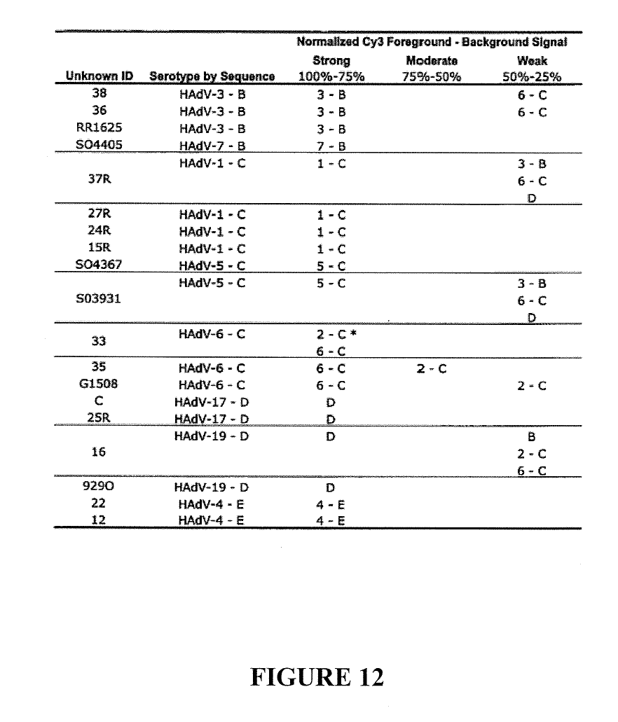

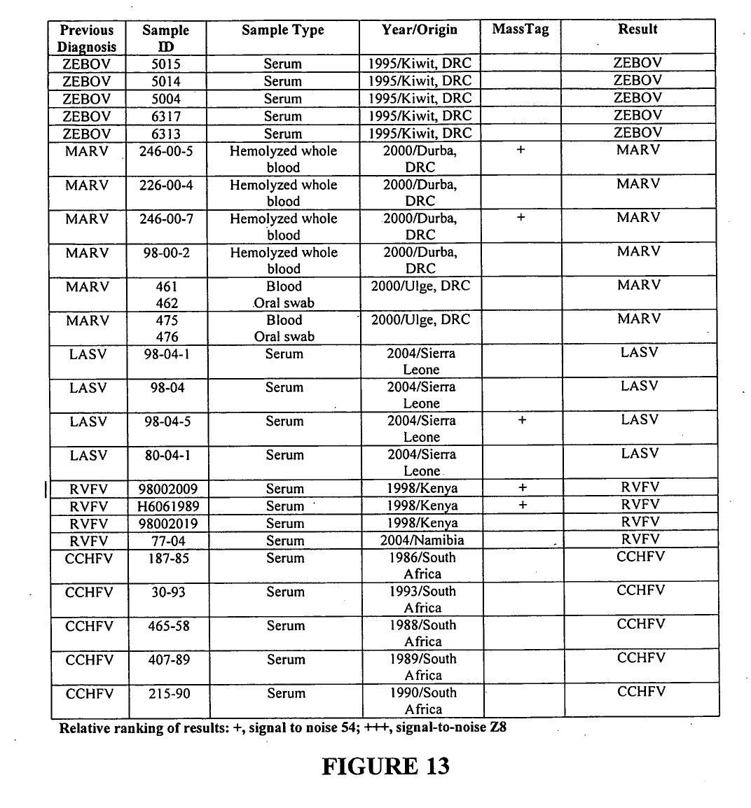

To assess the utility of the microarray in clinical microbiology, respiratory and fecal samples were collected during HAdV outbreaks in Spain. Nineteen samples representing species B, C, D, and E serotypes were processed for microarray and sequence analysis. Results using the two methods were concordant (FIG. 12).

The hexon gene array assay has major advantages with respect to classical methods for serotyping HAdV. The initial PCR detects known HAdV serotypes without nesting; furthermore, as it is based in highly conserved regions, and can be used for the discovery of new HAdV strains. Importantly for implementation in epidemiology and clinical microbiology, array based hybridization is inexpensive and suited to high throughput robotics.

### Example 8

### Differential Diagnosis of Viral Hemorrhagic Fever by MassTag PCR

Viral hemorrhagic fevers (VHF) are associated with high morbidity and mortality. Although current therapeutic options are limited, early differential diagnosis has implications for containment of contagion and may become important for clinical management. The invention provides a diagnostic system for rapid, multiplex PCR identification of 10 different causes of VHF.

Increasing international travel, trafficking in wildlife, political instability and terrorism have made emerging infectious diseases a global concern. Viral hemorrhagic fevers (VHF) warrant specific emphasis because of their high morbidity and mortality, and the potential for rapid dissemination through human-to-human transmission. The term ‘viral hemorrhagic fever’ characterizes a severe multisystem syndrome associated with fever, shock and a bleeding diathesis caused by infection with one of several RNA viruses, including Ebola and Marburg viruses (family Filoviridae), Lassa virus and the South American hemorrhagic fever viruses Guanarito, Junin, Machupo, and Sabia (Arenaviridae), Rift Valley fever, Crimean-Congo hemorrhagic fever, and hantaviruses (Bunyaviridae), and Kyasanur Forest disease, Omsk hemorrhagic fever, yellow fever, and dengue viruses (Flaviviridae). Although clinical management of VHFs is primarily supportive, early diagnosis is of utmost importance for containment of contagion and the implementation of public health measures, especially in instances where the agents are encountered out of their natural geographic context. Vaccines have been developed for yellow fever, Rift Valley fever, Junin, Kyasanur Forest disease, and hantaviruses, but only the yellow fever vaccine is widely available. Early treatment with immune plasma was shown to be effective in Junin virus infection. The nucleoside analogue ribavirin may be helpful if given early in the course of Lassa fever, Crimean-Congo hemorrhagic fever, or hemorrhagic fever with renal syndrome (HFRS), and is recommended in post-exposure prophylaxis and early treatment of arena- and bunyavirus infections.

Methods for direct detection of nucleic acids of microbial pathogens in clinical specimens are rapid, sensitive, and obviate the need for high level biocontainment associated in case of VHFs with cultivation based methods. Numerous systems are described for nucleic acid detection of VHF agents; however, none are multiplex. Although geographic location or travel history of suspected cases classically restricts the number of agents to be considered, diagnosis of VHF may be difficult in case of an intentional release, where such information will be lacking. Symptoms of VHF are initially nonspecific and may include fever, headache, myalgia, and gastrointestinal or upper respiratory tract complaints; thus, assays that allow simultaneous consideration of multiple agents are needed.

MassTag PCR is a multiplex assay wherein microbial gene targets are coded by a library of 64 distinct mass tags. Nucleic acids (RNA or DNA) are amplified by multiplex (RT)-PCR using up to 64 primers, each labeled via a photo-cleavable linkage with a different molecular weight tag. After separation of the amplification products from unincorporated primers, and release of the mass tags from the amplicons by UV-irradiation, tag identity is analyzed by mass spectrometry. The identity of the microbe in the clinical sample is determined by the presence of its two cognate tags, one from each primer.

To facilitate rapid differential diagnosis of VHF agents, the invention provides Greene MassTag Panel VHF v1.0 comprising the following targets: Ebola Zaire (ZEBOV), Ebola Sudan (SEBOV), Marburg (MARV), Lassa fever (LASV), Rift Valley fever (RVFV), Crimean-Congo hemorrhagic fever (CCHFV), Hantaan (HNTV), Seoul (SEOV), yellow fever (YFV), and Kyasanur Forest disease (KFDV) viruses. Oligonucleotide primers were designed in conserved genomic regions to detect the broadest number of members for a given pathogen species. A software program was developed that culls sequence information from GenBank, performs multiple alignments using ClustalW, and designs primers optimized for multiplex PCR. The program uses a greedy algorithm to identify conserved sequences and create the minimum set of primers for amplification of sequences in the alignment. Primers are selected within standard design constraints whenever possible (Tm=55° C.-65° C., GC content=40%-60%, no hairpins); degenerate positions are introduced in cases where template divergence requires more flexibility. Although degeneracy is not tolerated in the five 3′-nucleotides, MassTag PCR does allow up to four non-neighboring variable positions per primer. Primers are checked by BLAST for potential hybridization to sequenced vertebrate genomes (Table 12).

TABLE 12
Greene Mass Tag Panel VHF v1.0

| | Mass | | | | | |
|---|---|---|---|---|---|---|
| | Tag | Name | | Name | | |
| Target | FWD/REV | FWD | Sequence | REV | Sequence | Gene |
| ZEBOV | 718/646 | EboZa- | AACACCGGGTCTTAATTCTTATATCAA | EboZA- | GGTGGTAAAATTCCCATAGTAGTTCTTT | L |
| | | U234 | SEQ ID NO: 14 | L319 | SEQ ID NO: 15 | |
| SEBOV | 503/630 | EboSu- | CGAGCCTAACGTTTTGGGC | EboSU- | GCTCCAGGAATTGTTCGGGTA | L |
| | | U416 | SEQ ID NO: 16 | L489 | SEQ ID NO: 17 | |
| MARV | 654/395 | MAV- | CCCTCCATATCTTAGACAACATATTGTG | MARV- | CCCAACACTCCTGGTTCACAGC | L |
| | | U12816C | SEQ ID NO: 18 | L12994 | SEQ ID NO: 19 | |
| LASV * | 558/686 | Las4- | ACTGCATTYTCATACTTYCTRGAATC | Las4- | CCRGGYTTGACCAGTGCTGT | NP |
| | | U92 | SEQ ID NO: 20 | L257 | SEQ ID NO: 21 | |
| RVFV | 658/495 | RVF- | GGATTGACCTGTGCCTGTTGC | VP- | GCATTAGAAATGTCCTCTTTTGCTGC | N |
| | | U578 | SEQ ID NO: 22 | L660 | SEQ ID NO: 23 | |
| CCHFV | 499/710 | CCHV- | AGAAACACGTGCCGCTTACGCCCA | CCHV- | CCATTTCCYTTYTTRAACTCYTCAAACC | N |
| | | U4 | SEQ ID NO: 24 | L120 | SEQ ID NO: 25 | |
| HNTV | 479/702 | HAN- | AYACAGCAGCAGTTAGCCTCCT | HAN- | GCTGCCGTARGTAGTCCCTGTT | N |
| | | U179 | SEQ ID NO: 26 | L245 | SEQ ID NO: 27 | |
| SEOV | 455/602 | SEO- | CAGGATTGCAGCAGGGAAGA | SEOUL- | ATGATCACCAGGYTCTACCCC | N |
| | | U243 | SEQ ID NO: 27 | L309 | SEQ ID NO: 29 | |
| YFV | 467/670 | YF- | GCTGGGAGCGCGGTATC | YF- | GGAAGCCCAATGGTCCTCAT | NS5 |
| | | U186 | SEQ ID NO: 30 | L249 | SEQ ID NO: 31 | |
| KFDV | 483/14 | KYF- | TGGAAGCCTGGCTGAAAGAG | YF- | TCATCCCCACTGACCAGCAT | NS5 |
| | | U170 | SEQ ID NO: 32 | L233 | SEQ ID NO: 33 | |

Because only released mass tags will be analyzed, there is no need to stagger the size of amplification products created in multiplex reactions; thus, primers are selected for efficient and consistent performance irrespective of amplicon size (typically in the range of 80 to 200 base-pairs). Prior to committing to synthesis of tagged primers, the functionality of candidate multiplex primer panels is examined in a series of amplification reactions using prototype templates representing individual microbial targets. Primers that fail to yield a single, specific product-band in agarose gel analysis are replaced. Target sequence standards for evaluation are cloned into pCR2.1-TOPO (Invitrogen, Carlsbad, Calif.) using PCR amplification of cDNA templates obtained by reverse transcription of extracts from infected cultured cells, or by assembly of overlapping synthetic polynucleotides.

The agents assayed in the VHF panel have RNA genomes; thus, assay sensitivity was determined using synthetic RNA standards. Synthetic RNA standards were generated from linearized target sequence plasmids using T7 polymerase (mMessage mMachine, Invitrogen, Carlsbad, Calif.). After quantitation by UV spectrometry, RNA was serially diluted in 2.5 mg/ml yeast tRNA (Sigma), reverse transcribed with random hexamers using Superscript TI (Invitrogen, Carlsbad, Calif.), and analyzed by MassTag PCR as previously described (94° C. for 20 sec., 50° C. for 20 sec., 72° C. for 30 sec. with annealing temperature reduction in 1° C. increments between 65° C. to 51° C. and then 50° C. for 35 cycles; (14)). QIAquick 96 PCR purification cartridge (Qiagen, Hilden, Germany, with modified binding and wash buffers) were used to remove unincorporated primers before tags were decoupled from amplification products by UV photolysis in a flow cell, and analyzed in a single quadrapole mass spectrometer using positivemode atmospheric pressure chemical ionization (APCI) (Agilent Technologies, Palo Alto, Calif., USA). The sensitivity of the 10-plex VHF panel with synthetic RNA standards was 50 RNA copies or less per assay (Table 13).

TABLE 13
Sensitivity of detection using synthetic RNA standards

| | Detection Threshold |
|---|---|
| Pathogen | (RNA copies*) |
| ZEBOV | 20 |
| SEBOV | 20 |
| MARV | 20 |
| LASV | 20 |
| RVFV | 20 |
| CCHFV | 50 |
| HNTV | 20 |
| SEOV | 50 |
| YFV | 20 |
| KFDV | 20 |

*RNA copies refers to the number of molecules subjected to RT; half of the RT reaction was then used for PCR amplification

Tissue culture extracts were used to examine assay specificity. Random primed cDNA obtained from cultures of ZEBOV, SEBOV, MARV, YFV isolates from Gambia and Cote d'Ivoire, RVFV, CCHFV, HTNV, SEOV and LASV strains Josiah, NL, and AV were subjected to mass tag analysis. In all instances only the appropriate cognate mass tags were detected. No spurious signal was identified in assays with water or RNA controls.

Performance with clinical materials was tested using blood, sera or oral swaps from 24 human victims of VHF including 5 cases of Ebola hemorrhagic fever from the 1995 Kikwit outbreak, Democratic Republic of the Congo (DRC); 6 cases of Marburg hemorrhagic fever collected in 2000 during the Durba outbreak, DRC, and in 2005 in Uige, Angola; 4 cases of Lassa fever obtained in 2004 from Sierra Leone; 4 cases of Rift Valley fever from Namibia in 2004 and Kenya in 1998; and 5 cases of Crimean-Congo hemorrhagic fever from South Africa collected from 1986-93. Infection with the respective agent had been previously diagnosed through virus isolation, RT-PCR and in case of Lassa virus infections with antigen detection ELISA. Differential diagnosis by blinded MassTag PCR analysis was accurate in all cases (Table 14).

TABLE 14
Differential diagnosis by blinded MassTag PCR

| Previous | | | | MassTag |
|---|---|---|---|---|
| Diagnosis | Sample ID | Sample Type | Year/Origin | Result |
| ZEBOV | 5015 | serum | 1995/Kikwit, DRC | ZEBOV |
| ZEBOV | 5014 | serum | 1995/Kikwit, DRC | ZEBOV |
| ZEBOV | 5004 | serum | 1995/Kikwit, DRC | ZEBOV |
| ZEBOV | 6317 | serum | 1995/Kikwit, DRC | ZEBOV |
| ZEBOV | 6313 | serum | 1995/Kikwit, DRC | ZEBOV |
| MARV | 246-00-5 | hemolyzed whole blood | 2000/Durba, DRC | MARV |
| MARV | 226-00-4 | hemolyzed whole blood | 2000/Durba, DRC | MARV |
| MARV | 246-00-7 | hemolyzed whole blood | 2000/Durba, DRC | MARV |
| MARV | 98-00-2 | hemolyzed whole blood | 2000/Durba, DRC | MARV |
| MARV | 461 | blood | 2005/Uige, Angola | MARV |
| | 462 | oral swab | | |
| MARV | 475 | blood | 2005/Uige, Angola | MARV |
| | 476 | oral swab | | |
| LASV | 98-04-1 | serum | 2004/Sierra Leone | LASV |
| LASV | 98-04 | serum | 2004/Sierra Leone | LASV |
| LASV | 98-04-5 | serum | 2004/Sierra Leone | LASV |
| LASV | 80-04-1 | serum | 2004/Sierra Leone | LASV |
| RVFV | 98002009 | serum | 1998/Kenya | RVFV |
| RVFV | H6061989 | serum | 1998/Kenya | RVFV |
| RVFV | 98002019 | serum | 1998/Kenya | RVFV |
| RVFV | 77-04 | serum | 2004/Namibia | RVFV |
| CCHFV | 187-86 | serum | 1986/South Africa | CCHFV |
| CCHFV | 30-93 | serum | 1993/South Africa | CCHFV |
| CCHFV | 465-88 | serum | 1988/South Africa | CCHFV |
| CCHFV | 407-89 | serum | 1989/South Africa | CCHFV |
| CCHFV | 215-90 | serum | 1990/South Africa | CCHFV |

These results confirm earlier work in respiratory diseases indicating that MassTag PCR offers a rapid, sensitive, specific and economic approach to differential diagnosis of infectious diseases. Small, low-cost, or mobile APCI-MS units extend the applicability of this technique beyond selected reference laboratories. Given the capacity of the method to code for up to 32 genetic targets the hemorrhagic fever panel is being expanded to include additional viruses (dengue and South American hemorrhagic fever viruses). The inclusion of bacterial and parasitic agents is also being explored that may result in similar clinical presentations and, thus, have to be considered in differential diagnosis.

What is claimed:

1. A set of oligonucleotides for detecting vertebrate viruses, the set of oligonucleotides comprising a plurality of nucleic acid sequences that are reverse translated from at least about 10,000, about 20,000, about 30,000, about 40,000 or about 50,000 different amino acid sequences, each amino acid sequence comprising a motif conserved in a different virus family, genus, or species, wherein the virus family is selected from the group consisting of: Asfarviridae, Poxyiridae, Iridoviridae, Herpesviridae, Polydnaviridae, Papovaviridae, Adenoviridae, Circoviridae, Reoviridae, Birnaviridae, Orthomyxoviridae, Paramyxoviridae, Rhabdoviridae, Bornaviridae, Filoviridae, Arenaviridae, Retroviridae, Bunyaviridae, Caliciviridae, Picornaviridae, Astroviridae, Flaviviridae, Nodaviridae, Coronaviridae, Togaviridae, and Arteriviridae, and wherein the virus genus or species is belongs to one of the families.

2. A set of oligonucleotides for detecting vertebrate viruses, the set of oligonucleotides comprising a plurality of nucleic acid sequences that are reverse translated from at least about 10,000, about 20,000, about 30,000, about 40,000 or about 50,000 different amino acid sequences, each amino acid sequence comprising a motif conserved in a different virus genus, wherein the virus genus is selected from the group consisting of: Asfivirus, Orthopoxvirus, Parapoxvirus, Avipoxvirus, Capripoxvirus, Leporipoxvirus, Suipoxvirus, Molluscipoxvirus, Yatapoxvirus, Entomopoxvirus A, Entomopoxvirus B, Entomopoxvirus C, Iridovirus, Chloriridovirus, Ranavirus, Lymphocystivirus, Simplexvirus, Varicellovirus, Cytomegalovirus, Muromegalovirus, Roseolovirus, Lymphocryptovirus, Rhadinovirus, Ichnovirus, Bracovirus, Polyomavirus, Papillomavirus, Mastadenovirus, Aviadenovirus, Orthoreovirus, Orbivirus, Rotavirus, Coltivirus, Aquareovirus, Cypovirus, Fijivirus, Phytoreovirus, Oryzavirus, Aquabirnavirus, Avibirnavirus, Entomobirnavirus, Influenzavirus A, Influenzavirus B, Influenzavirus C, Influenzavirus D, Paramyxovirus, Morbillivirus, Rubulavirus, Pneumovirus, Bornavirus, Marburgvirus, Ebolavirus, Arenavirus, Alpharetrovirus, Betaretrovirus, Gammaretrovirus, Type D Retrovirus group, Deltaretrovirus, Epsilonretrovirus, Lentivirus, Spumavirus, Bunyavirus, Hantavirus, Nairovirus, Phlebovirus, Tospovirus, Calicivirus, Enterovirus, Rhinovirus, Hepatovirus, Cardiovirus, Aphthovirus, Astrovirus, Flavivirus, Pestivirus, Hepacivirus, Alphanodavirus, Coronavirus, Torovirus, Alphavirus, Arterivirus, and Deltavirus.

3. A set of oligonucleotides for detecting vertebrate viruses, the set of oligonucleotides comprising a plurality of nucleic acid sequences that are reverse translated from no more than one thousand different amino acid sequences, each amino acid sequence comprising a motif conserved in either:
(a) a virus family, wherein the virus family is selected from the group consisting of: Asfarviridae, Poxyiridae, Iridoviridae, Herpesviridae, Polydnaviridae, Papovaviridae, Adenoviridae, Circoviridae, Reoviridae, Birnaviridae, Orthomyxoviridae, Paramyxoviridae, Rhabdoviridae, Bornaviridae, Filoviridae, Arenaviridae, Retroviridae, Bunyaviridae, Caliciviridae, Picornaviridae, Astroviridae, Flaviviridae, Nodaviridae, Coronaviridae, Togaviridae, and Arteriviridae.
(b) a virus genus, wherein the virus genus is selected from the group consisting of: Asfivirus, Orthopoxvirus, Parapoxvirus, Avipoxvirus, Capripoxvirus, Leporipoxvirus, Suipoxvirus, Molluscipoxvirus, Yatapoxvirus, Entomopoxvirus A, Entomopoxvirus B, Entomopoxvirus C, Iridovirus, Chloriridovirus, Ranavirus, Lymphocystivirus, Simplexvirus, Varicellovirus, Cytomegalovirus, Muromegalovirus, Roseolovirus, Lymphocryptovirus, Rhadinovirus, Ichnovirus, Bracovirus, Polyomavirus, Papillomavirus, Mastadenovirus, Aviadenovirus, Orthoreovirus, Orbivirus, Rotavirus, Coltivirus, Aquareovirus, Cypovirus, Fijivirus, Phytoreovirus, Oryzavirus, Aquabirnavirus, Avibirnavirus, Entomobirnavirus, Influenzavirus A, Influenzavirus B, Influenzavirus C, Influenzavirus D, Paramyxovirus, Morbillivirus, Rubulavirus, Pneumovirus, Bornavirus, Marburgvirus, Ebolavirus, Arenavirus, Alpharetrovirus, Betaretrovirus, Gammaretrovirus, Type D Retrovirus group, Deltaretrovirus, Epsilonretrovirus, Lentivirus, Spumavirus, Bunyavirus, Hantavirus, Nairovirus, Phlebovirus, Tospovirus, Calicivirus, Enterovirus, Rhinovirus, Hepatovirus, Cardiovirus, Aphthovirus, Astrovirus, Flavivirus, Pestivirus, Hepacivirus, Alphanodavirus, Coronavirus, Torovirus, Alphavirus, Arterivirus, and Deltavirus; and/or
(c) a virus species from the virus family in (a) or the virus genus in (b);wherein the set of oligonucleotides as a whole can detect any virus that infects vertebrates.

4. The set of claim 3, wherein the amino acid sequences are selected from the group consisting of the amino acid sequences listed in the CD-ROM Table Appendix or amino acid sequences that are at least 10 residues in length and 90% identical to the amino acid sequences listed in the CD-ROM Table Appendix.

5. The set of claim 3, wherein each oligonucleotide in the set of oligonucleotides comprises a nucleotide sequence that is at least 20 nucleotides in length and has at least 90, 95, 96, 97, 98, or 99% sequence identity to a sequence selected from the group of sequences listed in the CD-ROM Table Appendix.

6. The set of any of claims 1, 2, or 3, wherein the motifs comprise an amino acid sequence from a viral polymerase or from a viral capsid.

7. The set of claim 1, further comprising oligonucleotides comprising a nucleotide sequence from a non-coding region of a genome of a vertebrate virus that is conserved in a vertebrate family, genus, or species.

8. The set of claim 2, further comprising oligonucleotides comprising a nucleotide sequence from a non-coding region of a genome of a vertebrate virus that is conserved in a vertebrate family, genus, or species.

9. The set of claim 3, further comprising oligonucleotides comprising a nucleotide sequence from a non-coding region of a genome of a vertebrate virus that is conserved in a vertebrate family, genus, or species.

10. A set of oligonucleotides for the detection of vertebrate viruses, the set of oligonucleotides comprising less than 10,000 different oligonucleotide sequences, wherein the set of oligonucleotides hybridizes to nucleic acid sequences from at least 10 viral species, wherein each oligonucleotide of the set comprises a nucleotide sequence reverse translated from an amino acid sequence listed in the CD-ROM Appendix Table.

11. A set of oligonucleotides for the detection of vertebrate viruses, the set of oligonucleotides comprising less than 10,000 different oligonucleotide sequences, wherein the set comprises a nucleotide sequence listed in the CD-ROM Appendix Table.

12. A set of oligonucleotides for the detection of vertebrate viruses, the set of oligonucleotides comprising less than 10,000 different oligonucleotide sequences, wherein the set comprises a nucleotide sequence complementary to a nucleotide sequence listed in the CD-ROM Appendix Table.

13. A method for designing an oligonucleotide for viral screening, the method comprising:
(a) compiling a database of viral sequences, wherein the database of viral sequences comprises nucleotide sequences and amino acid sequences representative of at least 10 different species of virus;
(b) classifying each nucleotide sequence and amino acid sequence into a viral order, family, genus, and species;
(c) identifying from the database of viral sequences a set of amino acid sequences wherein each amino acid sequence of the set comprises a protein domain or motif,
(d) identifying from the set of amino acid sequences of step (c) a subset of amino acid sequence motifs that are conserved throughout a viral family, genus, and/or species;
(e) determining the nucleotide sequences coding for the subset of amino acid sequence motifs of step (d), wherein the nucleotide sequences are obtained from the database of viral sequences; and
(f) designing a group of oligonucleotides comprising nucleotide sequences selected from the nucleotide sequences coding for the subset of amino acid sequence motifs.

14. The method of claim 13, wherein the designing of step (f) comprises using a set covering algorithm to determine a minimum number of sequences that needs to be selected from the nucleotide sequences coding for the subset of amino acid sequence motifs in order to represent every viral species in the viral database.

15. The method of claim 13, wherein in step (f), the group of oligonucleotides comprise nucleotide sequences selected from nucleotide sequences that code for amino acid sequence motifs conserved in a single viral family.

16. The method of claim 13, wherein in step (f), the group of oligonucleotides comprise nucleotide sequences selected from nucleotide sequences that code for amino acid sequence motifs conserved in a single viral genus.

17. The method of claim 13, wherein the viral sequence database consists essentially of sequences classified to be from vertebrate viruses.

18. The method of claim 13, wherein the viral sequence database does not comprise sequences from viruses that infect plants or bacteria.

19. The method of claim 13, wherein the compiling step comprises obtaining a nucleotide sequence or amino acid sequence identified to be viral from one or more public sequence collections, wherein the public sequence databases comprise GenBank®; DNA DataBank of Japan (DDBJ); the European Molecular Biology Laboratory (EMBL); Reference Sequence (RefSeq) collection; translated coding regions from DNA sequences in GenBank, EMBL, and DDBJ; Protein Information Resource (PIR); SWISS-PROT; Protein Research Foundation (PRF); and Protein Data Bank (PDB); and any successor entity.

20. The method of claim 13, wherein the viral database comprises sequences from at least 10 species of vertebrate viruses.

21. The method of claim 13, wherein the viral database comprises sequences for partial genomes of a viral species or for partial coding sequences for a viral protein.

22. The method of claim 13, wherein the nucleotide sequences for a viral species comprises sequences from more than one representative genome of the virus species.

23. The method of claim 13, wherein the classifying step further comprises classifying each nucleotide sequence and amino acid sequence into a viral subfamily, serogroup, subspecies, and/or isolate.

24. The method of claim 13, wherein the classifying step is based on viral taxonomic tree structure criteria from the International Committee on the Taxonomy of Viruses.

25. The method of claim 13, wherein the identifying in step (c) comprises using Hidden Markov Models (HMMs).

26. The method of claim 13, wherein step (d) comprises using a probabilistic model for identifying from the set of amino acid sequences of step (c) the subset of amino acid sequence motifs that are conserved throughout a viral family, genus, and/or species.

27. The method of claim 25, wherein the probabilistic model is a MEME algorithm.

28. A microarray comprising any one of the oligonucleotides of any of claims 1-3, 5, 7, 8, 9, 10, 11 or 12.

29. A method for identifying a virus from a environmental or clinical sample, the method comprising:
(a) isolating nucleic acids from a sample containing the virus;
(b) labeling the nucleic acids with a label;
(c) hybridizing the labeled nucleic acids to a set of oligonucleotides of any of claims 1-3, 5, 7, 8, 9, 10, 11 or 12; and
(d) identifying the nucleic acids from the set of nucleic acids of any of claims 1-3, 5, 7, 8, 9, 10, 11 or 12 that hybridized to the labeled nucleic acids, thereby identifying the virus.

30. A computer program product residing on a computer readable medium, the computer program product comprising instructions for causing a computer to:
(a) compile a database of viral sequences, wherein the database of viral sequences comprises nucleotide sequences and amino acid sequences representative of at least 10 different species of virus;
(b) classify each nucleotide sequence and amino acid sequence into a viral order, family, genus, and species;
(c) identify from the database of viral sequences a set of amino acid sequences wherein each amino acid sequence of the set comprises a protein domain;
(d) identify from the set of amino acid sequences of step (c) a subset of amino acid sequence motifs that are conserved throughout a viral family, genus, and/or species;
(e) determine the nucleotide sequences coding for the subset of amino acid sequence motifs of step (d), wherein the nucleotide sequences are obtained from the database of viral sequences; and
(f) design a group of oligonucleotides comprising nucleotide sequences selected from the nucleotide sequences coding for the subset of amino acid sequence motifs.

31. A method for designing one or more primers, wherein the method comprises:
(a) generating a similarity matrix of multiple nucleic acid sequence sub-alignments within a nucleic acid sequence alignment by pairwise comparison with a tree structure building,
(b) generating a phylogenetic tree of nodes from the similarity matrix of step (a) by hierarchical clustering, wherein each node comprises a one or more nucleic acid sequences in a sub-alignment,
(c) identifying one or more nucleic acid sequences in each node of step (b) by scoring on the basis of one or more parameters,
(d) determining a minimum number of nucleic acid sequences identified in step (c) capable of amplifying the nucleic acid sequence in the subalignment of step (b) with a set covering algorithm, and
(e) identifying nucleic acid sequences that are capable of forming primer pairs on the basis of one or more parameters.

32. The method of claim 31, wherein the tree structure building algorithm comprises:
(a) a method of extracting sub-alignments from an entire alignment,
(b) a method of filtering sub-alignments for uniqueness, and
(c) a method of performing a pairwise comparison of sub-alignments.

33. The method of claim 31, wherein the hierarchical clustering algorithm is based on Euclidean distance.

34. The method of claim 31, wherein the parameters measured by the scoring function comprises: melting temperature, GC content, homopolymeric runs, hairpin/primer dimer formation, degeneracy, ability to hybridize to a template, total mismatches to a template.

35. The method of claim 31, wherein the set covering algorithm is a greedy algorithm.

36. The method of claim 31, wherein the an parameters used to identify nucleic acid sequences capable of forming primer pairs comprise the length of an amplicon or melting temperature differences between nucleic acids.

37. The method of claim 31, wherein the pairs can encode a viral amino acid sequence.

38. A method of designing a database of coding viral oligonucleotides, wherein the method comprises:
(a) compiling a database of a plurality of viral nucleic acid sequences,
(b) compiling a database of a plurality of viral protein sequences,
(c) identifying a subset of viral nucleic acid sequences in the database of step (a) capable of encoding one or more amino acid sequences having at least 90% sequence identity to any viral protein sequence in the viral protein database of step (b), wherein nucleic acid sequences in the subset of viral nucleic acid sequences comprise oligonucleotides having a length from about 10 to about 250 nucleotides, about 20 to about 65 nucleotides or about 25 to about 60 nucleotides,
(d) translating the oligonucleotides of step (c) to generate a database of back-translated viral protein sequences,
(e) identifying amino acid sequences in the back translated viral protein sequences of step
(d) that share at least 60% identity with conserved eukaryotic, viral and bacterial protein domains, wherein the identification is made with a Hidden Markov model algorithm with an algorithm for pairwise comparison of homologous clusters,
(f) identifying the viral nucleic acid sequences in the database of step (a) that are capable of encoding amino acid sequences identified in step (e),
(g) identifying nucleic acid sequences that are statistically overrepresented in the viral nucleic acid sequences of step (f), wherein the identification is made with a probabilistic model algorithm,
(h) identifying oligonucleotides from nucleic acid sequences in step (g) that are suitable for hybridization,
(i) compiling the oligonucleotides identified in step (h) into a database of viral oligonucleotides, wherein the database is a database of coding viral oligonucleotides.

39. A method of designing a database of degenerate coding viral oligonucleotides, wherein the method comprises:
(a) compiling a database of a plurality of viral nucleic acid sequences,
(b) compiling a database of a plurality of viral protein sequences,
(c) identifying a subset of viral nucleic acid sequences in the database of step (a) capable of encoding one or more amino acid sequences having at least 90% sequence identity to any viral protein sequence in the viral protein database of step (b), wherein nucleic acid sequences in the subset of viral nucleic acid sequences comprise oligonucleotides having a length from about 10 to about 250 nucleotides, about 20 to about 65 nucleotides or about 25 to about 60 nucleotides,
(d) translating the oligonucleotides of step (c) to generate back-translated viral protein sequences,
(e) identifying amino acid sequences in the back translated viral protein sequences of step (d) that share at least 60% identity with conserved eukaryotic, viral and bacterial protein domains, wherein the identification is made with a Hidden Markov model algorithm with an algorithm for pairwise comparison of homologous clusters,
(f) identifying a minimum set of degenerate nucleotides sequences that are capable of encoding the amino acid sequences identified in step (e)
(g) identifying nucleic acid sequences that are statistically overrepresented in the viral nucleic acid sequences of step (f), wherein the identification is made with a probabilistic model algorithm,
(h) identifying oligonucleotides from nucleic acid sequences in step (g) that are suitable for hybridization,
(i) compiling the oligonucleotides identified in step (h) into a database of viral oligonucleotides, wherein the database is a database of degenerate coding viral oligonucleotides.

40. A method of designing a database of non-coding viral oligonucleotides, wherein the method comprises:
(a) compiling a database of a plurality of viral nucleic acid sequences,
(b) compiling a database of a plurality of viral protein sequences,
(c) identifying a subset of viral nucleic acid sequences in the database of step (a) that are not capable of encoding one or more proteins having at least 80% sequence identity any viral protein sequence in the viral protein database of step (b), wherein nucleic acids sequences in the subset of viral nucleic acid sequences comprise oligonucleotides having a length from about 10 to about 250 nucleotides, about 20 to about 65 nucleotides or about 25 to about 60 nucleotides,
(d) identifying nucleic acid sequences that are statistically overrepresented in the viral nucleic acid sequences of step (f), wherein the identification is made with a probabilistic model algorithm,
(e) identifying oligonucleotides from nucleic acid sequences in step (g) that are suitable for hybridization,
(f) compiling the oligonucleotides identified in step (h) into a database of viral oligonucleotides, wherein the database is a database of non-coding viral oligonucleotides.

41. The method of any of claims 31, 38-40, wherein the oligonucleotides suitable for an oligonucleotide-related application.

42. The method of claim 41, wherein the oligonucleotide related application comprises microarray screening, PCR and RNAi analysis.

43. A method for identifying sequence patterns that are conserved across viral taxa or within a viral taxon, wherein the method comprises steps (a) through (g) of claim 38.

44. A method for identifying sequence patterns that are conserved across viral taxa or within a viral taxon, wherein the method comprises steps (a) through (g) of claim 39.

45. A method for identifying sequence patterns that are conserved across viral taxa or within a viral taxon, wherein the method comprises steps (a) through (d) of claim 40.

46. The method of any of claim 43-45, wherein the sequence patterns are nucleic acid motifs, amino acid motifs or protein domains.

47. A method for generating conserved peptides that can be used as immunogens for the generation of an antibody against a virus, wherein one or more oligonucleotides of any of claims 1-3, 5, 7, 8, 9, 10, 11, 12, 13, 15, 16, 30, 38, 39 or 40 are translated to produce immunogens for the generation of antibodies.

48. A computer-readable medium containing computer-executable instructions that, when executed by a processor, cause the processor to perform a method for designing an oligonucleotide for viral screening, wherein the method comprises the method of any of claims 13, 38, 39 or 40.

49. A computer-readable medium for storing data for access by an application, comprising: a tree structure stored in the computer-readable medium, wherein the tree structure comprises nodes connected by edges, wherein at least one of the nodes is a top node describing a viral nucleic acid sequence, and wherein the top node has a least two child nodes describing a viral nucleic acid sequence, wherein the at least two child nodes are generated by the method of claim 32.

50. The computer-readable medium for storing data for access by an application of claim 49, wherein at least one of the nodes in the tree structure correspond to a viral family, genus or species.

51. A system for mapping a viral nucleic acid sequence to a tree structure, wherein the system comprises: an interface; a memory containing the tree structure of claim 50; and a processor in communication with the memory and the interface; wherein the processor:
(a) receives nucleic acid hybridization information from the interface,
(b) receives instructions from the memory, wherein the instructions from the memory comprise instructions that when executed by the processor cause the processor to map the nucleic acid hybridization information of step (a) to at least one node of tree structure of claim 50 and generate an output,
(c) sends the output the interface.

52. The system of claim 51, wherein the interface is in communication with a network.

53. The system of claim 51, wherein the nucleic acid hybridization information is from a micro array.

54. The system of claim 51, wherein the nucleic acid hybridization information comprises a pattern of positive signals.

55. The system of claim 51, wherein the processor receives instructions from the memory to:
(a) analyze a pattern of positive signals in the hybridization information,
(b) eliminate signals from internal controls and position makers, and
(c) calculate a probability that the pattern of positive signals matches a viral family, genus, or species.

56. A system for at least one of diagnosis, surveillance, or discovery of infection or disease, the system comprising:
(a) a processor,
(b) a storage device coupled to the processor,
(c) a database of any of claim 1-3 residing on the storage device, wherein the processor checks a database for new genetic information and updates the database with the genetic information, and
(d) an input device coupled to the processor which inputs a genetic sequence and hybridization results of the genetic sequence, wherein the processor analyzes the hybridization results of the genetic sequence and generates information regarding the placement of the genetic sequence in the database.

57. A method for updating a database of genetic information, comprising:
(a) obtaining one or more sequences from at least one source of information at an interval,
(b) reconciling differences among the one or more sequences obtained in step (a) and sequences in a database of any of claim 1-3,
(c) determining if the one or more sequences should be added to the database, and
(d) adding the one or more sequences in the database, where the genetic information in the database is updated.

58. The method of claim 57, wherein the determining comprises determining if the sequence is covered, within a programmable difference of nucleotide mismatches, by at least one sequence already in the database.

59. A viral detection kit comprising the microarray of claim 28.
